(19) United States
(12) Patent Application Publication
(10) Pub. No.: US 2012/0178765 A1
(43) Pub. Date: Jul. 12, 2012

(54) NOVEL GLUCOKINASE ACTIVATORS AND PROCESSES FOR THE PREPARATION THEREOF

(75) Inventors: Won-Hui Yi, Suwon-si (KR); Tae-Dong Han, Yongin-si (KR); Koo-Yeon Lee, Yongin-si (KR); Young-Hwan Kim, Yongin-si (KR); Eun-Hye Jung, Seocho-gu (KR); Dong-Hoon Lee, Suwon-si (KR); Yoo-Hoi Park, Hwaseong-si (KR); Kyung-Nan Min, Seocho-gu (KR); Jae-Gyu Kim, Suwon-si (KR); Byung-Kyu Lee, Gunpo-si (KR)

(73) Assignee: YUHAN CORPORATION, Seoul (KR)

(21) Appl. No.: 13/497,219

(22) PCT Filed: Sep. 17, 2010
(86) PCT No.: PCT/KR2010/006415

§ 371 Date: Mar. 20, 2012

(30) Foreign Application Priority Data
Sep. 22, 2009 (KR) 10-2009-0089824

(51) Int. Cl. A61K 31/4965; C07D 231/40; C07D 241/20; A61P 3/10; A61K 31/426; A61K 31/415; A61K 31/455; C07D 277/46; C07D 213/80

(52) U.S. Cl. 514/255.06; 548/195; 548/372.5; 544/336; 546/309; 514/371; 514/407; 514/352

(57) ABSTRACT

The present invention provides an amide derivative having a stilbene or 1,2-diphenylethane moiety within the molecule thereof, a process for the preparation thereof, and a pharmaceutical composition comprising the same. The amide derivative of the present invention activates glucokinase remarkably, and therefore they can be usefully applied for treating glucokinase-mediated diseases, such as hyperglycemia and diabetes.

### TECHNICAL FIELD

The present invention relates to a novel glucokinase activator, more specifically a novel compound or its pharmaceutically acceptable salt activating glucokinase, a process for the preparation thereof, and a pharmaceutical composition comprising the same.

### BACKGROUND ART

Glucokinase, one of the hexokinases, catalyzes phosphorylation of glucose to glucose-6-phosphate, which is the first step in glucose metabolism (Alexander M. Efanov, David G. Barrett et al., Endocrinology, 146, 3696-3701, 2007). Glucokinase plays an important role in maintaining glucose homeostasis in the body, through direct control of glucose level in the blood.

Glucokinase is mainly expressed in pancreatic β-cells and hepatocytes. Glucokinase in the pancreatic β-cells serves as an enzyme controlling the rate of glucose catabolism, so as to induce glucose-dependent insulin secretion. And also, glucokinase in the hepatocytes induces glucose uptake and glycogen synthesis. Therefore, glucokinase plays an important role in the regulation of blood glucose level (D. Zelent, H. Najafi, S. Odili, C. Buettger, H. Weik-Collins, C. Li, N. Doliba, J. Grimsby, F. M. Matschinsky, Biochemical Society Transactions, 33, 306-310, 2005).

It has been reported that, while glucokinase-deficient mice show severe hyper-glycemia, the mice transplanting a gene encoding the glucokinase show reduction in basal blood glucose level; as well as resistance against diabetes induced by feeding with a hyperlipidemic diet. That is, it has been verified in animal models that there is existed close relationship between glucokinase and diabetes. From these reports, it is evident that glucokinase serves as an excellent glucose sensor for maintaining glucose homeostasis and that an agent for increasing the activity of glucokinase can be developed as an anti-diabetic agent.

Glucokinase may exist in three conformations, i.e., in open form, in super-open form, or in closed form. Through the conformational changes, glucokinase rotates slow or rapid catalytic cycles. When glucokinase exists in the closed form, the allosteric pocket in glucokinase becomes a suitable form for binding with an agent activating glucokinase, i.e., glucokinase activator (GKA) (Sarabu, R., Taub, R., Grimsby, J., Drug Discovery Today Therapeutic Strategies, 4, 111-115, 2007). That is, a GKA binds with the allosteric pocket in glucokinase to cause any conformational change in glucokinase, thereby stabilizing the closed form of glucokinase, which results in activating glucokinase so as to catalyze metabolism of the substrate, i.e., glucose (Grimsby, J., Sarabu, R., Corbett, W. L., Haynes, N. E., Bizzaro, F. T., Coffey, J. W., Guertin, K. R., Hilliard, D. W., Kester, R. F. and Mahaney, P. E., Science, 301, 370-373, 2003). Therefore, a GKA can selectively act on the glucokinase having such an allosteric pocket, while not activating other hexokinases.

From the fact that GKAs act on pancreatic β-cells and hepatocytes affecting glucose homeostasis to facilitate insulin-secretion and glucose-metabolism, various researches are being made for developing novel GKAs as an anti-type II diabetic agent. The currently known GKAs can be classified into ‘carbon’-centered GKAs, aromatic ring-centered GKAs, amino acid-based GKAs and their analogues, according to their chemical structures (Sarabu, R., Berthel, S. J., Kester, R. F., Tilley, J., W., Expert Opin. Ther. Patents, 18, 759-768, 2008; Matschinsky, F. M., Magnuson, M. A., Eds., In Frontiers in Diabetes, 16, 145-154, 2004; Kamata, K., Mitsuya, M., Nishimura, T., Eiki, J.-i., Nagata, Y., Structure, 12, 429, 2004; WO03/097824; WO08/075,073). And also, WO03/000267, WO03/015774, WO07/125,103, WO07/125,105, etc. disclose benzamide derivatives as a glucokinase modulator.

### DISCLOSURE OF INVENTION

### Technical Problem

The present inventors found that an amide derivative having a stilbene or 1,2-diphenylethane moiety within the molecule thereof activates glucokinase remarkably, and therefore that the derivative is useful for treating glucokinase-mediated diseases, such as hyperglycemia and diabetes.

Therefore, the present invention provides the above novel compound or its pharmaceutically acceptable salt activating glucokinase, a process for the preparation thereof, and a pharmaceutical composition comprising the same. And also, the present invention provides an intermediate useful for preparing the compound or its pharmaceutically acceptable salt.

### Solution to Problem

According to an aspect of the present invention, there is provided a novel compound or its pharmaceutically acceptable salt, which has a blood glucose level-reducing activity through activating glucokinase.

According to another aspect of the present invention, there is provided a process for preparing the novel compound or its pharmaceutically acceptable salt.

According to still another aspect of the present invention, there is provided a novel intermediate useful for preparing the compound or its pharmaceutically acceptable salt.

According to still another aspect of the present invention, there is provided a pharmaceutical composition comprising the compound or its pharmaceutically acceptable salt as an active ingredient.

### Advantageous Effects of Invention

The compounds of the present invention, i.e., the amide derivatives having a stilbene or 1,2-diphenylethane moiety within the molecule thereof activates glucokinase remarkably, and therefore they can be usefully applied for treating glucokinase-mediated diseases, such as hyperglycemia and diabetes.

### BEST MODE FOR CARRYING OUT THE INVENTION

As used herein, the term “heteroaryl” or “heteroaryl ring” refers to a 5- or 6-membered monocyclic heteroaryl group having 1 to 3 hetero atoms selected from nitrogen (N) atom, oxygen (O) atom, and sulfur (S) atom. And also, the term “heteroaryl” or “heteroaryl ring” includes a bicyclic heteroaryl formed by fusing the 5- or 6-membered monocyclic heteroaryl with benzene or pyridine. For example, the monocyclic heteroaryl (or heteroary ring) includes thiazole, pyrazole, oxazole, imidazole, pyrrole, furan, thiophene, isothiazole, isoxazole, triazole, thiadiazole, tetrazole, oxadiazole, triazine, pyridine, pyridazine, pyrimidine, pyrazine, etc. And also, the bicyclic heteroaryl (or heteroary ring) includes benzothiazole, bezoxazole, benzimidazole, benzofuran, benzothiophene, benzisoxazole, indole, indoline, quinoline, isoquinoline, quinazoline, imidazopyridine, oxazolopyridine, etc.

The term “aryl” or “aryl ring” refers to a functional group derived from an aromatic ring with delocalized pi electron clouds. The “aryl” or “aryl ring” includes, for example, a C₆-C₁₂ hydrocarbon-ring such as phenyl, naphthyl, and biphenyl.

The term “alkyl” refers to a straight or branched aliphatic hydrocarbon radical. For example, C₁-C₆ alkyl means a straight or branched aliphatic hydrocarbon having 1 to 6 carbon atoms, such as methyl, ethyl, propyl, n-butyl, n-pentyl, n-hexyl, isopropyl, isobutyl, sec-butyl, tert-butyl, neopentyl, and isopentyl.

The term “alkoxy” refers to a radical formed by substituting the hydrogen atom of a hydroxyl group with an alkyl. For example, C₁-C₆ alkoxy includes methoxy, ethoxy, propoxy, n-butoxy, n-pentyloxy, isopropoxy, sec-butoxy, tert-butoxy, neopentyloxy, and isopentyloxy.

The present invention provides a compound of Formula 1 or its pharmaceutically acceptable salt:

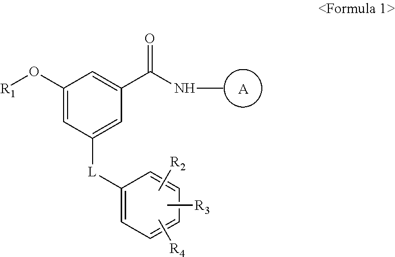

wherein,

L is —CH₂═CH₂— or —CH₂—CH₂—,

A is a heteroaryl ring having 1 to 3 hetero atoms selected from nitrogen (N) atom and sulfur (S) atom, wherein the heteroaryl ring is optionally substituted with one or more substituents selected from the group consisting of C₁-C₆ alkyl, hydroxycarbonyl, C₁-C₆ alkoxycarbonyl, and halogen,

R₁ is a C₁-C₆ alkyl group optionally substituted with C₁-C₆ alkoxy,

R₂, R₃, and R₄ is, independently each other, hydrogen; a C₁-C₆ alkyl group; a C₁-C₆ alkoxy group; halogen; nitro; amino; or —NH—R₅, with the proviso that R₂, R₃, and R₄ cannot be hydrogen at the same time,

R₅ is —C(O)—R₆, —C(O)—O—R₆, —C(O)—NH—R₆, —C(S)—NH—R₆, or —SO₂—R₆, and

R₆ is selected from the group consisting of

a C₁-C₆ alkyl group;

a C₁-C₆ alkoxycarbonyl-C₁-C₆ alkyl group;

a hydroxycarbonyl-C₁-C₆ alkyl group;

an aryl group optionally substituted with one or more substituents selected from the group consisting of nitro, halogen, and C₁-C₆ alkoxy;

an aryl-C₁-C₆ alkyl group;

a 5- or 6-membered heteroaryl ring; and

a 5- to 14-membered heteroaryl-C₁-C₆ alkyl.

In the compound of Formula 1 or its pharmaceutically acceptable salt, preferably A is a heteroaryl ring selected from the group consisting of thiazolyl, pyridyl, pyrazolyl, and pyrazinyl, wherein the heteroaryl ring is optionally substituted with one or more substituents selected from the group consisting of C₁-C₆ alkyl, hydroxycarbonyl, C₁-C₆ alkoxycarbonyl, and halogen.

And also, in the compound of Formula 1 or its pharmaceutically acceptable salt, preferably,

R₂, R₃, and R₄ is, independently each other, hydrogen; a C₁-C₆ alkyl group; a C₁-C₆ alkoxy group; halogen; nitro; amino; or —NH—R₅, with the proviso that R₂, R₃, and R₄ cannot be hydrogen at the same time,

R₅ is —C(O)—R₆, —C(O)—O—R₆, —C(O)—NH—R₆, —C(S)—NH—R₆, or —SO₂—R₆, and

R₆ is selected from the group consisting of

a C₁-C₆ alkyl group;

a C₁-C₆ alkoxycarbonyl-C₁-C₃ alkyl group;

a hydroxycarbonyl-C₁-C₃ alkyl group;

a phenyl group optionally substituted with one or more substituents selected from the group consisting of nitro, halogen, and C₁-C₃ alkoxy;

a phenyl-C₁-C₃ alkyl group;

a 5- or 6-membered heteroaryl ring; and

a 5- or 6-membered heteroaryl-C₁-C₃ alkyl.

The compound of Formula 1 or its pharmaceutically acceptable salt may be in the form of cis- or trans-geometrical isomer, via the double bond therein (i.e., L). The compound of Formula 1 or its pharmaceutically acceptable salt comprises both cis- and trans-geometrical isomers. And also, the compound of Formula 1 or its pharmaceutically acceptable salt may have substituents containing asymmetric carbon (for example, the substituent R₁) and therefore be in the form of racemic mixture (RS) or in forms of optical isomers, such as (R) or (S) isomer. The compound of Formula 1 or its pharmaceutically acceptable salt comprises both racemic mixture (RS) and optical isomers such as (R) or (S) isomer.

Examples of preferable compounds of Formula 1 or their pharmaceutically acceptable salts are:
3-[trans-2-(p-tolyl)vinyl]-5-isobutoxy-N-(thiazol-2-yl)-benzamide;
3-[trans-2-(4-fluorophenyl)vinyl]-5-isobutoxy-N-(thiazol-2-yl)-benzamide;
3-[trans-2-(2-nitrophenyl)vinyl]-5-isobutoxy-N-(thiazol-2-yl)-benzamide;
3-[trans-2-(3,4-dimethoxyphenyl)vinyl]-5-isobutoxy-N-(thiazol-2-yl)-benzamide;
3-[trans-2-(2,3-dimethoxyphenyl)vinyl]-5-isobutoxy-N-(thiazol-2-yl)-benzamide;
3-[trans-2-(2-fluorophenyl)vinyl]-5-(2-methoxy-(1S)-methyl-ethoxy)-N-(thiazol-2-yl)-benzamide;
3-[trans-2-(3-fluorophenyl)vinyl]-5-(2-methoxy-(1S)-methyl-ethoxy)-N-(thiazol-2-yl)-benzamide;
3-[trans-2-(4-fluorophenyl)vinyl]-5-(2-methoxy-(1S)-methyl-ethoxy)-N-(thiazol-2-yl)-benzamide;
3-[trans-2-(2,3-difluorophenyl)vinyl]-5-(2-methoxy-(1S)-methyl-ethoxy)-N-(thiazol-2-yl)-benzamide;
3-[trans-2-(2,4-difluorophenyl)vinyl]-5-(2-methoxy-(1S)-methyl-ethoxy)-N-(thiazol-2-yl)-benzamide;
3-[trans-2-(2,5-difluorophenyl)vinyl]-5-(2-methoxy-(1S)-methyl-ethoxy)-N-(thiazol-2-yl)-benzamide;
3-[trans-2-(2,6-difluorophenyl)vinyl]-5-(2-methoxy-(1S)-methyl-ethoxy)-N-(thiazol-2-yl)-benzamide;
3-[trans-2-(2-nitrophenyl)vinyl]-5-(2-methoxy-(1S)-methyl-ethoxy)-N-(thiazol-2-yl)-benzamide;
3-[trans-2-(3-nitrophenyl)vinyl]-5-(2-methoxy-(1S)-methyl-ethoxy)-N-(thiazol-2-yl)-benzamide;
3-[trans-2-(4-nitrophenyl)vinyl]-5-(2-methoxy-(1S)-methyl-ethoxy)-N-(thiazol-2-yl)-benzamide;
3-[trans-2-(2-methoxyphenyl)vinyl]-5-(2-methoxy-(1S)-methyl-ethoxy)-N-(thiazol-2-yl)-benzamide;
3-[trans-2-(2,3-dimethoxyphenyl)vinyl]-5-(2-methoxy-(1S)-methyl-ethoxy)-N-(thiazol-2-yl)-benzamide;
3-[trans-2-(2,3,5-trifluorophenyl)vinyl]-5-(2-methoxy-(1S)-methyl-ethoxy)-N-(thiazol-2-yl)-benzamide;
3-[trans-2-(2-nitrophenyl)vinyl]-5-(2-methoxy-(1S)-methyl-ethoxy)-N-(5-fluorothiazol-2-yl)-benzamide;
3-[trans-2-(3-nitrophenyl)vinyl]-5-(2-methoxy-(1S)-methyl-ethoxy)-N-(5-fluorothiazol-2-yl)-benzamide;
3-[trans-2-(4-nitrophenyl)vinyl]-5-(2-methoxy-(1S)-methyl-ethoxy)-N-(5-fluorothiazol-2-yl)-benzamide;
3-[trans-2-(2-fluorophenyl)vinyl]-5-(2-methoxy-(1S)-methyl-ethoxy)-N-(5-fluorothiazol-2-yl)-benzamide;
3-[trans-2-(4-fluorophenyl)vinyl]-5-(2-methoxy-(1S)-methyl-ethoxy)-N-(5-fluorothiazol-2-yl)-benzamide;
3-[trans-2-(2,3-difluorophenyl)vinyl]-5-(2-methoxy-(1S)-methyl-ethoxy)-N-(5-fluorothiazol-2-yl)-benzamide;
3-[trans-2-(2,4-difluorophenyl)vinyl]-5-(2-methoxy-(1S)-methyl-ethoxy)-N-(5-fluorothiazol-2-yl)-benzamide;
3-[trans-2-(2,5-difluorophenyl)vinyl]-5-(2-methoxy-(1S)-methyl-ethoxy)-N-(5-fluorothiazol-2-yl)-benzamide;
3-[trans-2-(2,3,5-trifluorophenyl)vinyl]-5-(2-methoxy-(1S)-methyl-ethoxy)-N-(5-fluorothiazol-2-yl)-benzamide;
3-[trans-2-(2,3-dimethoxyphenyl)vinyl]-5-(2-methoxy-(1S)-methyl-ethoxy)-N-(5-fluorothiazol-2-yl)-benzamide;
3-[trans-2-(2,6-dimethoxyphenyl)vinyl]-5-(2-methoxy-(1S)-methyl-ethoxy)-N-(5-flu orothiazol-2-yl)-benzamide;
3-[trans-2-(3,4-dimethoxyphenyl)vinyl]-5-(2-methoxy-(1S)-methyl-ethoxy)-N-(5-flu orothiazol-2-yl)-benzamide;
3-[trans-2-(3,5-dimethoxyphenyl)vinyl]-5-(2-methoxy-(1S)-methyl-ethoxy)-N-(5-flu orothiazol-2-yl)-benzamide;
3-[trans-2-(2-nitrophenyl)vinyl]-5-(2-methoxy-(1S)-methyl-ethoxy)-N-(1-methyl-1H-pyrazol-3-yl)-benzamide;
3-[trans-2-(2-fluorophenyl)vinyl]-5-(2-methoxy-(1S)-methyl-ethoxy)-N-(1-methyl-1H-pyrazol-3-yl)-benzamide;
3-[trans-2-(2,3-difluorophenyl)vinyl]-5-(2-methoxy-(1S)-methyl-ethoxy)-N-(1-methy l-1H-pyrazol-3-yl)-benzamide;
3-[trans-2-(2,4-difluorophenyl)vinyl]-5-(2-methoxy-(1S)-methyl-ethoxy)-N-(1-methy l-1H-pyrazol-3-yl)-benzamide;
3-[trans-2-(2,5-difluorophenyl)vinyl]-5-(2-methoxy-(1S)-methyl-ethoxy)-N-(1-methy l-1H-pyrazol-3-yl)-benzamide;
3-[trans-2-(2,6-difluorophenyl)vinyl]-5-(2-methoxy-(1S)-methyl-ethoxy)-N-(1-methy l-1H-pyrazol-3-yl)-benzamide;
3-[trans-2-(3-nitrophenyl)vinyl]-5-(2-methoxy-(1S)-methyl-ethoxy)-N-(1-methyl-1H-pyrazol-3-yl)-benzamide;
3-[trans-2-(4-fluorophenyl)vinyl]-5-(2-methoxy-(1S)-methyl-ethoxy)-N-(1-methyl-1H-pyrazol-3-yl)-benzamide;
3-[trans-2-(3,5-dimethoxyphenyl)vinyl]-5-(2-methoxy-(1S)-methyl-ethoxy)-N-(1-me thyl-1H-pyrazol-3-yl)-benzamide;
3-[trans-2-(4-methoxyphenyl)vinyl]-5-(2-methoxy-(1S)-methyl-ethoxy)-N-(1-methyl-1H-pyrazol-3-yl)-benzamide;
3-[trans-2-(2-methoxyphenyl)vinyl]-5-(2-methoxy-(1S)-methyl-ethoxy)-N-(1-methyl-1H-pyrazol-3-yl)-benzamide;
3-[trans-2-(2-bromophenyl)vinyl]-5-(2-methoxy-(1S)-methyl-ethoxy)-N-(1-methyl-1H-pyrazol-3-yl)-benzamide;
3-[trans-2-(3-bromophenyl)vinyl]-5-(2-methoxy-(1S)-methyl-ethoxy)-N-(1-methyl-1H-pyrazol-3-yl)-benzamide;
3-[trans-2-(4-bromophenyl)vinyl]-5-(2-methoxy-(1S)-methyl-ethoxy)-N-(1-methyl-1H-pyrazol-3-yl)-benzamide;
3-[trans-2-(2-chlorophenyl)vinyl]-5-(2-methoxy-(1S)-methyl-ethoxy)-N-(1-methyl-1H-pyrazol-3-yl)-benzamide;
3-[trans-2-(3-chlorophenyl)vinyl]-5-(2-methoxy-(1S)-methyl-ethoxy)-N-(1-methyl-1H-pyrazol-3-yl)-benzamide;
3-[trans-2-(2-methoxyphenyl)vinyl]-5-(2-methoxy-(1S)-methyl-ethoxy)-N-(pyrazin-2-yl)-benzamide;
3-[trans-2-(4-methoxyphenyl)vinyl]-5-(2-methoxy-(1S)-methyl-ethoxy)-N-(pyrazin-2-yl)-benzamide;
3-[trans-2-(3,5-dimethoxyphenyl)vinyl]-5-(2-methoxy-(1S)-methyl-ethoxy)-N-(pyraz in-2-yl)-benzamide;
3-[trans-2-(2-fluorophenyl)vinyl]-5-(2-methoxy-(1S)-methyl-ethoxy)-N-(pyrazin-2-y l)-benzamide;
3-[trans-2-(4-fluorophenyl)vinyl]-5-(2-methoxy-(1S)-methyl-ethoxy)-N-(pyrazin-2-y l)-benzamide;
3-[trans-2-(2-chlorophenyl)vinyl]-5-(2-methoxy-(1S)-methyl-ethoxy)-N-(pyrazin-2-y l)-benzamide;
3-[trans-2-(3-chlorophenyl)vinyl]-5-(2-methoxy-(1S)-methyl-ethoxy)-N-(pyrazin-2-y l)-benzamide;
3-[trans-2-(2,3-difluorophenyl)vinyl]-5-(2-methoxy-(1S)-methyl-ethoxy)-N-(pyrazin-2-yl)-benzamide;
3-[trans-2-(2,4-difluorophenyl)vinyl]-5-(2-methoxy-(1S)-methyl-ethoxy)-N-(pyrazin-2-yl)-benzamide;
3-[trans-2-(2,5-difluorophenyl)vinyl]-5-(2-methoxy-(1S)-methyl-ethoxy)-N-(pyrazin-2-yl)-benzamide;
3-[trans-2-(2,6-difluorophenyl)vinyl]-5-(2-methoxy-(1S)-methyl-ethoxy)-N-(pyrazin-2-yl)-benzamide;
3-[trans-2-(3-bromophenyl)vinyl]-5-(2-methoxy-(1S)-methyl-ethoxy)-N-(pyrazin-2-y l)-benzamide;
3-[trans-2-(4-bromophenyl)vinyl]-5-(2-methoxy-(1S)-methyl-ethoxy)-N-(pyrazin-2-y l)-benzamide;
3-[trans-2-(2-methoxyphenyl)vinyl]-5-(2-methoxy-(1S)-methyl-ethoxy)-N-(nicotinic acid-3-methyl ester-6-yl)-benzamide;
3-[trans-2-(2-nitrophenyl)vinyl]-5-(2-methoxy-(1S)-methyl-ethoxy)-N-(nicotinic acid-3-methyl ester-6-yl)-benzamide;
3-[trans-2-(3-nitrophenyl)vinyl]-5-(2-methoxy-(1S)-methyl-ethoxy)-N-(nicotinic acid-3-methyl ester-6-yl)-benzamide;
3-[trans-2-(2-fluorophenyl)vinyl]-5-(2-methoxy-(1S)-methyl-ethoxy)-N-(nicotinic acid-3-methyl ester-6-yl)-benzamide;
3-[trans-2-(4-fluorophenyl)vinyl]-5-(2-methoxy-(1S)-methyl-ethoxy)-N-(nicotinic acid-3-methyl ester-6-yl)-benzamide;
3-[trans-2-(2,3-difluorophenyl)vinyl]-5-(2-methoxy-(1S)-methyl-ethoxy)-N-(nicotini c acid-3-methyl ester-6-yl)-benzamide;
3-[trans-2-(2,4-difluorophenyl)vinyl]-5-(2-methoxy-(1S)-methyl-ethoxy)-N-(nicotini c acid-3-methyl ester-6-yl)-benzamide;
3-[trans-2-(2,5-difluorophenyl)vinyl]-5-(2-methoxy-(1S)-methyl-ethoxy)-N-(nicotini c acid-3-methyl ester-6-yl)-benzamide;
3-[trans-2-(2,6-difluorophenyl)vinyl]-5-(2-methoxy-(1S)-methyl-ethoxy)-N-(nicotini c acid-3-methyl ester-6-yl)-benzamide;
3-[trans-2-(2-fluorophenyl)vinyl]-5-isobutoxy-N-(thiazol-2-yl)-benzamide;
3-[trans-2-(2,5-difluorophenyl)vinyl]-5-isobutoxy-N-(thiazol-2-yl)-benzamide;
3-[trans-2-(2,6-difluorophenyl)vinyl]-5-isobutoxy-N-(thiazol-2-yl)-benzamide;
3-[trans-2-(2-methoxyphenyl)vinyl]-5-(2-methoxy-(1S)-methyl-ethoxy)-N-(nicotinic acid-6-yl)-benzamide;
3-[trans-2-(2-nitrophenyl)vinyl]-5-(2-methoxy-(1S)-methyl-ethoxy)-N-(nicotinic acid-6-yl)-benzamide;
3-[trans-2-(3-nitrophenyl)vinyl]-5-(2-methoxy-(1S)-methyl-ethoxy)-N-(nicotinic acid-6-yl)-benzamide;
3-[trans-2-(2-fluorophenyl)vinyl]-5-(2-methoxy-(1S)-methyl-ethoxy)-N-(nicotinic acid-6-yl)-benzamide;
3-[trans-2-(4-fluorophenyl)vinyl]-5-(2-methoxy-(1S)-methyl-ethoxy)-N-(nicotinic acid-6-yl)-benzamide;
3-[trans-2-(2,3-difluorophenyl)vinyl]-5-(2-methoxy-(1S)-methyl-ethoxy)-N-(nicotini c acid-6-yl)-benzamide;
3-[trans-2-(2,4-difluorophenyl)vinyl]-5-(2-methoxy-(1S)-methyl-ethoxy)-N-(nicotini c acid-6-yl)-benzamide;
3-[trans-2-(2,5-difluorophenyl)vinyl]-5-(2-methoxy-(1S)-methyl-ethoxy)-N-(nicotini c acid-6-yl)-benzamide;
3-[trans-2-(2,6-difluorophenyl)vinyl]-5-(2-methoxy-(1S)-methyl-ethoxy)-N-(nicotini c acid-6-yl)-benzamide;
3-[trans-2-(2-methoxyphenyl)vinyl]-5-(2-methoxy-(1S)-methyl-ethoxy)-N-(5-fluorot hiazol-2-yl)-benzamide;
3-[trans-2-(2-methoxyphenyl)vinyl]-5-(2-methoxy-(1S)-methyl-ethoxy)-N-(1-methyl-1H-pyrazol-3-yl)-benzamide;
3-[2-(2-fluorophenyl)ethyl]-5-(2-methoxy-(1S)-methyl-ethoxy)-N-(thiazol-2-yl)-ben zamide;
3-[2-(2,4-difluorophenyl)ethyl]-5-(2-methoxy-(1S)-methyl-ethoxy)-N-(thiazol-2-yl)-benzamide;
3-[2-(2,5-difluorophenyl)ethyl]-5-(2-methoxy-(1S)-methyl-ethoxy)-N-(thiazol-2-yl)-benzamide;
3-[2-(2,6-difluorophenyl)ethyl]-5-(2-methoxy-(1S)-methyl-ethoxy)-N-(thiazol-2-yl)-benzamide;
3-[2-(2-aminophenyl)ethyl]-5-(2-methoxy-(1S)-methyl-ethoxy)-N-(thiazol-2-yl)-ben zamide;
3-[2-(2-methoxyphenyl)ethyl]-5-(2-methoxy-(1S)-methyl-ethoxy)-N-(thiazol-2-yl)-b enzamide;
3-[2-(2,3-dimethoxyphenyl)ethyl]-5-(2-methoxy-(1S)-methyl-ethoxy)-N-(thiazol-2-y l)-benzamide;
3-[2-(3-aminophenyl)ethyl]-5-(2-methoxy-(1S)-methyl-ethoxy)-N-(thiazol-2-yl)-ben zamide;
3-[2-(4-aminophenyl)ethyl]-5-(2-methoxy-(1S)-methyl-ethoxy)-N-(thiazol-2-yl)-ben zamide;
3-[2-(2-methoxyphenyl)ethyl]-5-(2-methoxy-(1S)-methyl-ethoxy)-N-(1-methyl-1H-p yrazol-3-yl)-benzamide;
3-[2-(2-fluorophenyl)ethyl]-5-(2-methoxy-(1S)-methyl-ethoxy)-N-(1-methyl-1H-pyr azol-3-yl)-benzamide;
3-[2-(2,3-difluorophenyl)ethyl]-5-(2-methoxy-(1S)-methyl-ethoxy)-N-(1-methyl-1H-pyrazol-3-yl)-benzamide;
3-[2-(2,4-difluorophenyl)ethyl]-5-(2-methoxy-(1S)-methyl-ethoxy)-N-(1-methyl-1H-pyrazol-3-yl)-benzamide;
3-[2-(3-aminophenyl)ethyl]-5-(2-methoxy-(1S)-methyl-ethoxy)-N-(1-methyl-1H-pyr azol-3-yl)-benzamide;
3-[2-(4-fluorophenyl)ethyl]-5-(2-methoxy-(1S)-methyl-ethoxy)-N-(1-methyl-1H-pyr azol-3-yl)-benzamide;
3-[2-(3,5-dimethoxyphenyl)ethyl]-5-(2-methoxy-(1S)-methyl-ethoxy)-N-(1-methyl-1H-pyrazol-3-yl)-benzamide;
3-[2-(4-methoxyphenyl)ethyl]-5-(2-methoxy-(1S)-methyl-ethoxy)-N-(1-methyl-1H-p yrazol-3-yl)-benzamide;
3-[2-(2-chlorophenyl)ethyl]-5-(2-methoxy-(1S)-methyl-ethoxy)-N-(1-methyl-1H-pyr azol-3-yl)-benzamide;
3-[2-(3-chlorophenyl)ethyl]-5-(2-methoxy-(1S)-methyl-ethoxy)-N-(1-methyl-1H-pyr azol-3-yl)-benzamide;
3-[2-(2,3-dimethoxyphenyl)ethyl]-5-(2-methoxy-(1S)-methyl-ethoxy)-N-(1-methyl-1H-pyrazol-3-yl)-benzamide;
3-[2-(2,4-dimethoxyphenyl)ethyl]-5-(2-methoxy-(1S)-methyl-ethoxy)-N-(1-methyl-1H-pyrazol-3-yl)-benzamide;
3-[2-(2,4-dimethoxyphenyl)ethyl]-5-(2-methoxy-(1S)-methyl-ethoxy)-N-(pyrazin-2-yl)-benzamide;
3-[2-(2-methoxyphenyl)ethyl]-5-(2-methoxy-(1S)-methyl-ethoxy)-N-(pyrazin-2-yl)-b enzamide;
3-[2-(2-fluorophenyl)ethyl]-5-(2-methoxy-(1S)-methyl-ethoxy)-N-(pyrazin-2-yl)-ben zamide;
3-[2-(4-fluorophenyl)ethyl]-5-(2-methoxy-(1S)-methyl-ethoxy)-N-(pyrazin-2-yl)-ben zamide;
3-[2-(2,6-difluorophenyl)ethyl]-5-(2-methoxy-(1S)-methyl-ethoxy)-N-(nicotinic acid-6-yl)-benzamide;
3-[2-(2-aminophenyl)ethyl]-5-(2-methoxy-(1S)-methyl-ethoxy)-N-(thiazol-2-yl)-ben zamide hydrochloride;
3-[2-(3-aminophenyl)ethyl]-5-(2-methoxy-(1S)-methyl-ethoxy)-N-(thiazol-2-yl)-ben zamide hydrochloride;
3-[2-(4-aminophenyl)ethyl]-5-(2-methoxy-(1S)-methyl-ethoxy)-N-(thiazol-2-yl)-ben zamide hydrochloride;
3-[2-(2-methanesulfonylaminophenyl)ethyl]-5-(2-methoxy-(1S)-methyl-ethoxy)-N-(t hiazol-2-yl)-benzamide;
3-{2-[2-(2-thiophen-2-yl)acetylamino)phenyl]ethyl}-5-(2-methoxy-(1S)-methyl-etho xy)-N-(thiazol-2-yl)-benzamide;
3-[2-(2-benzenesulfonylaminophenyl)ethyl]-5-(2-methoxy-(1S)-methyl-ethoxy)-N-(t hiazol-2-yl)-benzamide;
3-[2-(2-acetylaminophenyl)ethyl]-5-(2-methoxy-(1S)-methyl-ethoxy)-N-(thiazol-2-yl)-benzamide;
3-{2-[2-(carbamic acid ethyl ester)phenyl]ethyl}-5-(2-methoxy-(1S)-methyl-ethoxy)-N-(thiazol-2-yl)-benzamide;
3-[2-(3-acetylaminophenyl)ethyl]-5-(2-methoxy-(1S)-methyl-ethoxy)-N-(thiazol-2-yl)-benzamide;
3-[2-(3-butyrylaminophenyl)ethyl]-5-(2-methoxy-(1S)-methyl-ethoxy)-N-(thiazol-2-yl)-benzamide;
3-{2-[3-(3-methyl-butyrylamino)phenyl]ethyl}-5-(2-methoxy-(1S)-methyl-ethoxy)-N-(thiazol-2-yl)-benzamide;
3-{2-[3-(malonamic acid ethyl ester)phenyl]ethyl}-5-(2-methoxy-(1S)-methyl-ethoxy)-N-(thiazol-2-yl)-benzamide;
3-{2-[3-(carbamic acid ethyl ester)phenyl]ethyl}-5-(2-methoxy-(1S)-methyl-ethoxy)-N-(thiazol-2-yl)-benzamide;
3-{2-[3-(phenylacetylamino)phenyl]ethyl}-5-(2-methoxy-(1S)-methyl-ethoxy)-N-(thi azol-2-yl)-benzamide;
3-[2-(3-benzoylaminophenyl)ethyl]-5-(2-methoxy-(1S)-methyl-ethoxy)-N-(thiazol-2-yl)-benzamide;
3-{2-[3-(4-fluorobenzoylamino)phenyl]ethyl}-5-(2-methoxy-(1S)-methyl-ethoxy)-N-(thiazol-2-yl)-benzamide;
3-{2-[3-(4-chlorobenzoylamino)phenyl]ethyl}-5-(2-methoxy-(1S)-methyl-ethoxy)-N-(thiazol-2-yl)-benzamide;
3-{2-[3-(4-nitrobenzoylamino)phenyl]ethyl}-5-(2-methoxy-(1S)-methyl-ethoxy)-N-(t hiazol-2-yl)-benzamide;
3-[2-(3-isonicotinamidephenyl)ethyl]-5-(2-methoxy-(1S)-methyl-ethoxy)-N-(thiazol-2-yl)-benzamide;
3-{2-[3-(2-(thiophen-2-yl)acetylamino)phenyl]ethyl}-5-(2-methoxy-(1S)-methyl-eth oxy)-N-(thiazol-2-yl)-benzamide;
3-{2-[3-(3-phenyl-ureido)phenyl]ethyl}-5-(2-methoxy-(1S)-methyl-ethoxy)-N-(thiaz ol-2-yl)-benzamide;
3-{2-[3-(3-ethyl-thio-ureido)phenyl]ethyl}-5-(2-methoxy-(1S)-methyl-ethoxy)-N-(thi azol-2-yl)-benzamide;
3-[2-(4-acetylaminophenyl)ethyl]-5-(2-methoxy-(1S)-methyl-ethoxy)-N-(thiazol-2-yl)-benzamide;
3-[2-(4-butyrylaminophenyl)ethyl]-5-(2-methoxy-(1S)-methyl-ethoxy)-N-(thiazol-2-yl)-benzamide;
3-{2-[4-(3-methyl-butyrylamino)phenyl]ethyl}-5-(2-methoxy-(1S)-methyl-ethoxy)-N-(thiazol-2-yl)-benzamide;
3-{2-[4-(malonamic acid ethyl ester)phenyl]ethyl}-5-(2-methoxy-(1S)-methyl-ethoxy)-N-(thiazol-2-yl)-benzamide;
3-{2-[4-(carbamic acid ethyl ester)phenyl]ethyl}-5-(2-methoxy-(1S)-methyl-ethoxy)-N-(thiazol-2-yl)-benzamide;
3-{2-[4-(phenylacetylamino)phenyl]ethyl}-5-(2-methoxy-(1S)-methyl-ethoxy)-N-(thi azol-2-yl)-benzamide;
3-[2-(4-benzoylaminophenyl)ethyl]-5-(2-methoxy-(1S)-methyl-ethoxy)-N-(thiazol-2-yl)-benzamide;
3-{2-[4-(4-fluorobenzoylamino)phenyl]ethyl}-5-(2-methoxy-(1S)-methyl-ethoxy)-N-(thiazol-2-yl)-benzamide;
3-{2-[4-(4-chlorobenzoylamino)phenyl]ethyl}-5-(2-methoxy-(1S)-methyl-ethoxy)-N-(thiazol-2-yl)-benzamide;
3-{2-[4-(3,5-dimethoxybenzoylamino)phenyl]ethyl}-5-(2-methoxy-(1S)-methyl-etho xy)-N-(thiazol-2-yl)-benzamide;
3-[2-(4-isonicotinamidephenyl)ethyl]-5-(2-methoxy-(1S)-methyl-ethoxy)-N-(thiazol-2-yl)-benzamide;
3-{2-[4-(2-(thiophen-2-yl)acetylamino)phenyl]ethyl}-5-(2-methoxy-(1S)-methyl-eth oxy)-N-(thiazol-2-yl)-benzamide;
3-[2-(4-benzenesulfonylaminophenyl)ethyl]-5-(2-methoxy-(1S)-methyl-ethoxy)-N-(t hiazol-2-yl)-benzamide;
3-{2-[4-(3-phenyl-ureido)phenyl]ethyl}-5-(2-methoxy-(1S)-methyl-ethoxy)-N-(thiaz ol-2-yl)-benzamide;
3-{2-[4-(3-ethyl-thio-ureido)phenyl]ethyl}-5-(2-methoxy-(1S)-methyl-ethoxy)-N-(thi azol-2-yl)-benzamide;
3-[2-(3-malonamic acid phenyl)ethyl]-5-(2-methoxy-(1S)-methyl-ethoxy)-N-(thiazol-2-yl)-benzamide;
3-[trans-2-(4-fluorophenyl)vinyl]-5-(1-methoxymethyl-propoxy)-N-(thiazol-2-yl)-be nzamide.

The compound of Formula 1 of the present invention may be in a pharmaceutically acceptable salt form. The salt may be an acid addition salt form, which includes e.g., salts derived from an inorganic acid such as hydrochloric acid, hydrobromic acid, sulfuric acid, sulfamic acid, phosphoric acid, or nitric acid; and salts derived from an organic acid such as acetic acid, propionic acid, succinic acid, glycolic acid, stearic acid, citric acid, maleic acid, malonic acid, methanesulfonic acid, tartaric acid, malic acid, phenylacetic acid, glutamic acid, benzoic acid, salicylic acid, 2-acetoxybenzoic acid, fumaric acid, p-toluenesulfonic acid, oxalic acid or trifluoroacetic acid. And also, the pharmaceutically acceptable salt may be a metal salt form, which includes e.g., salts derived from a metal such as lithium, sodium, potassium, magnesium, or calcium. The acid addition salts or metal salts may be prepared according to conventional methods.

The present invention provides process for preparing a compound of Formula 1a or its pharmaceutically acceptable salt, which comprises reacting a compound of Formula 2 with a compound of Formula 3:

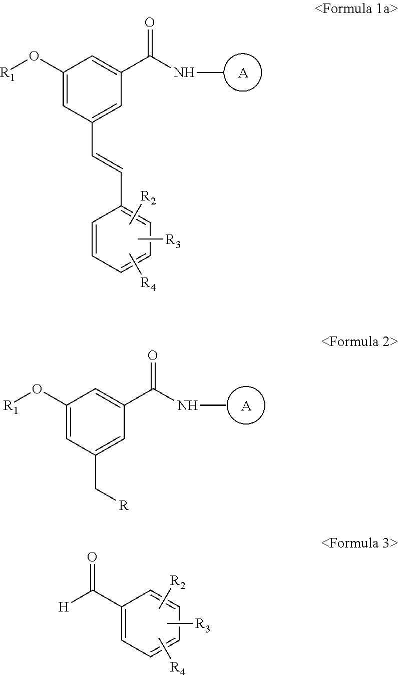

wherein, A, R₁, R₂, R₃, and R₄ are the same as defined in the above; R is —P(O)(OR′)₂ or triphenylphosphonium (—PPh₃); and R′ is a C₁-C₆ alkyl group or an aryl group.

Specifically, the compound of Formula 1a may be prepared via Wittig reaction using the compound of Formula 2 and the aldehydes of Formula 3. The reaction may be carried out using an inorganic base such as potassium hydroxide, potassium carbonate, potassium tert-butoxide, sodium hydride, butyllithium or sodium bis(trimethylsilyl)amide. And also, an organic solvent such as dichloromethane, tetrahydrofuran, diethyl ether, 1,2-dimethoxyethane, methyl tert-butyl ester, N,N-dimethylformamide or toluene may be used as a solvent. Typically, the reaction may be carried out in a temperature ranging from −78° C. to room temperature. Other reaction conditions, including e.g., reaction time, may be determined from the reaction conditions for conventional Wittig reactions (Barbara Czako and Laszlo Kurti, STRATEGIC APPLICATIONS of NAMED REACTIONS in ORGANIC SYNTHESIS, 2005).

The compound of Formula 3 is commercially available. The compound of Formula 2 may be prepared, e.g., according to the following Reaction Scheme 1.

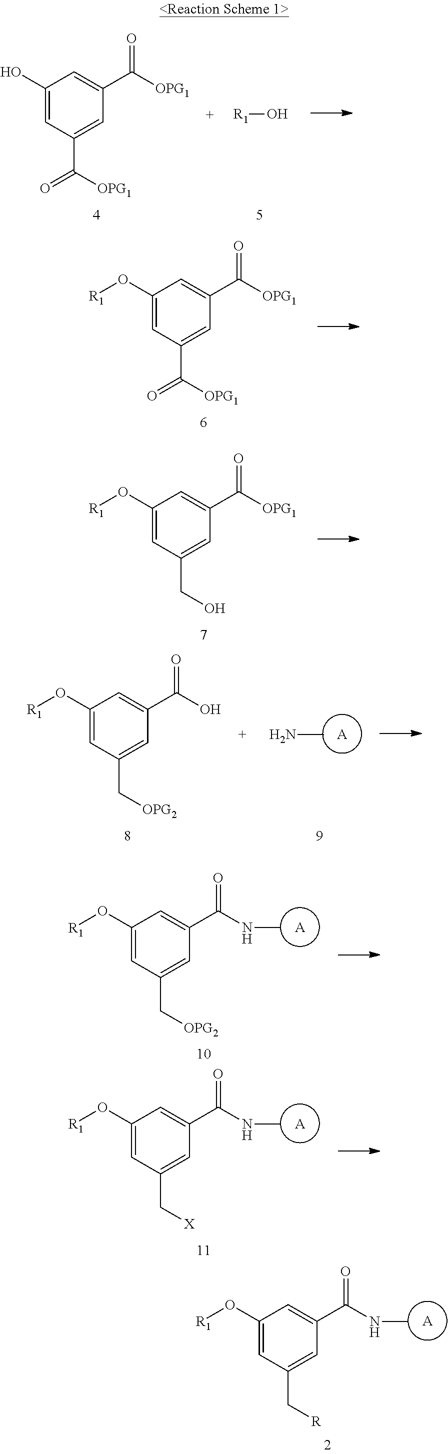

In the above Reaction Scheme 1, A, R₁ and R are the same as defined in the above; PG₁ is a carboxyl-protecting group; PG₂ is a hydroxyl-protecting group; and X is a halogen.

Specifically, the compound of Formula 4 is reacted with the compound of Formula 5 to obtain the compound of Formula 6. The compounds of Formula 4 and Formula 5 are commercially available. The reaction of the compound of Formula 4 and the compound of Formula 5 may be carried out according to Mitzunobu reaction, using diethyl azodicarboxylate (DEAD) or diisopropyl azodicarboxylate (DIAD) in the presence of triphenylphosphine or tri-n-butylphosphine. And also, a polar organic solvent such as dichloromethane, dioxane or tetrahydrofuran may be used as a solvent. Typically, the reaction may be carried out at a temperature ranging from 0° C. to room temperature. In certain circumstances, the reaction may be carried out at higher temperature. Other reaction conditions, including e.g., reaction time, may be determined from the reaction conditions for conventional Mitzunobu reactions (Barbara Czako and Laszlo Kurti, STRATEGIC APPLICATIONS of NAMED REACTIONS in ORGANIC SYNTHESIS, 2005).

A selective deprotection of the carboxyl-protecting group (PG₁) in the compound of Formula 6 and then a reduction of the resulting deprotected carboxylic acid may give the compound of Formula 7. The carboxyl-protecting group (PG₁) may be a lower alkyl (i.e., C₁-C₆ alkyl) group such as methyl, ethyl, isobutyl, or tert-butyl. Typically, the PG₁ deprotection may be carried out using an inorganic base, e.g., sodium hydroxide, lithium hydroxide or potassium hydroxide. In the selective deprotection of the carboxyl-protecting group (PG₁), since PG₁ is symmetric, two PG₁ may show the same reactivity. However, the use of 1 equivalent of a deprotecting agent may deprotect only one of the carboxyl-protecting groups. In the selective deprotection, water or a mixture of water and a polar solvent (e.g., tetrahydrofuran, alcohols) may be used as a solvent. The deprotection reaction may be carried out at a temperature ranging from room temperature to 50° C. Through the subsequent reduction, only the resulting deprotected carboxylic acid is reduced, without reducing the carboxyl-protecting group still having PG₁ protecting group. The reduction may be carried out at a temperature ranging from 0° C. to room temperature, using a tetrahydrofuran-borane complex (Huan, Zhenwei; Landgrebe, John A.; Peterson, Kimberly, Tetrahedron Letters, 24, 2829-2832, 1983).

A deprotection of the remaining carboxyl-protecting group (PG₁) and an introduction of a hydroxyl-protecting group (PG₂) may give the compound of Formula 8. The PG₁ deprotection may be carried out according to the same conditions as in preparing the compound of Formula 7. The hydroxyl-protecting group (PG₂) may be conventional hydroxyl-protecting groups, such as acetoxy, tert-butyldimethylsilyl, benzoyl or methoxymethyl ether. The deprotection of the carboxyl group and the introduction of the hydroxyl-protecting group are carried out according to conventional methods (Theodora W. Greene and Peter G. M. Wuts, Protective groups in organic synthesis, 3rd Ed., 1999). For example, the hydroxyl-protecting reaction may be carried out in a mixed solvent of dichloromethane and water, at room temperature, using an organic base such as pyridine.

The compound of Formula 8 may be coupled with the commercially available amine compound of Formula 9 to convert to the compound of Formula 10. The coupling, i.e., amide-coupling may be carried out according to conventional methods, for example, an acyl halide method, an azide method, a carboxylic acid anhydride method, a carbodiimide method, an active ester method, or a carbonyldiimidazole method (see Miklos Bodanszky, Principles of Peptide Synthesis, 2nd Ed., 1993). Preferably, the carbodiimide method or the acyl halide method may be used.

The coupling reaction according to the carbodiimide method may be carried out using dicyclohexylcarbodiimide (DCC), diisopropylcarbodiimide, or soluble N-(3-dimethylaminopropyl)-N′-ethylcarbodiimide (EDAC). If necessary, the reaction may be facilitated by adding 1-hydroxybenzotriazole (HOBT) thereto. The coupling reaction may be carried out in an inert solvent such as dichloromethane, acetonitrile or N,N-dimethylformamide, in the presence of an organic base such as triethylamine, diisopropylethylamine, N-methylmorpholine, N,N-dimethylaminopyridine or N-methylpyrrolidine, at a temperature ranging from room temperature to 50° C.

The coupling reaction according to the acyl halide method may be carried out by reacting the compound of Formula 8 with thionyl chloride or oxalyl chloride to convert the carboxylic acid to an acyl halide and then reacting the resulting acyl halide with the compound of Formula 9 in the presence of an organic base such as pyridine, tri-ethylamine, diisopropyl ethylamine, N-methylmorpholine, N,N-dimethylaminopyridine or N-methylpyrrolidine. The coupling reaction may be carried out in a solvent such as dichloromethane or pyridine, at a temperature ranging from room temperature to 100° C.

The compound of Formula 10 may be converted to the compound of Formula 11 by carrying out deprotection of the hydroxyl protecting group and then halogenation. The deprotection of the hydroxyl protecting group may be carried out according to conventional methods (Theodora W. Greene, Peter G. M. Wuts, Protective groups in organic synthesis, 3rd Ed., 1999). For example, the deprotection of the hydroxyl protecting group (PG₂) may be carried out in water or a mixed solvent of water and a polar solvent such as tetrahydrofuran or alcohols, at a temperature ranging from room temperature to 50° C., using an inorganic base such as sodium hydroxide, lithium hydroxide or potassium hydroxide. Alternatively, according to substituent(s) of the ring A, potassium carbonate may be also used as an inorganic base and a mixed solvent of water and methanol may be used. The halogenation is preferably a bromination. For example, the bromination may be carried out by introducing bromine to the hydroxyl group obtained from the deprotection, using e.g., tribromo phosphate (Hill, Dale H.; Parvez, Masood A.; Sen, Ayusman, J. Am. Chem. Soc., 116, 2889-2901, 1994). Typically, the bromination may be carried out at a temperature ranging from 0° C. to room temperature.

The compound of Formula 11 may be converted to the compound of Formula 2 through phosphite-substitution or triphenylphosphine-substitution reaction.

The phosphate-substitution reaction may be carried out by nucleophilic substitution reaction of the compound of Formula 11 with trialkyl phosphite (Gronowitz, Salo; Stenhammar, Karin; Svensson, Leif, Heterocycles, 15, 947-959, 1981). The reaction may be in the presence of or in the absence of a solvent. In case of carrying out the reaction in the presence of a solvent, the solvent includes dichloromethane or toluene. Typically, the reaction may be carried out at a temperature ranging from room temperature to 160° C. The triphenylphosphine-substitution reaction may be carried out by reacting the compound of Formula 11 with triphenylphosphine, in the presence of a base such as butyllithium, sodium hydride or sodium tert-butoxide. The reaction may be carried out in a nonpolar solvent such as benzene or toluene, at about 100° C. (Filler, R.; Heffern, E. W., Journal of Organic Chemistry, 32, 3249-3251, 1967).

The present invention provides a process for preparing a compound of Formula 1a or its pharmaceutically acceptable salt, which comprises reacting a compound of Formula 4 with a compound of Formula 5:

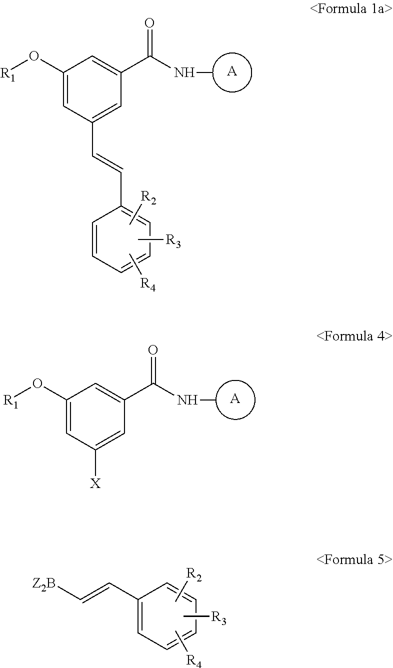

wherein, A, R₁, R₂, R₃, and R₄ are the same as defined in the above; X is halogen, O-trifluoromethanesulfonyl, or —OP(O)(OR′)₂; R′ is a C₁-C₆ alkyl group or an aryl group; and Z is hydroxy, a C₁-C₆ alkyl group, or a O—C₁-C₆ alkyl group.

Specifically, the compound of Formula 1a may be prepared according to Suzuki reaction, using the compound of Formula 4 and the compound of Formula 5. The reaction may be carried out using a palladium catalyst. The palladium catalyst includes, for example, palladium diacetate (Pd(OAc)₂), tris(dibenzylideneacetone)dipalladium (Pd₂(dba)₃), tetrakis(triphenylphosphine)palladium (Pd(PPh₃)₄) or palladium di[1,1′-bis(diphenylphosphino)ferrocene]dichloride (PdCl₂(dppf)₂). In carrying out the reaction, a ligand and a base may be also added thereto, in addition to the palladium catalyst. The ligand includes, for example, (S)-2,2-bis(diphenylphosphino)-1,1-binaphthyl (BINAP), 1,1′-bis(diphenylphosphino)ferrocene (dppf) or tri-O-tolylphosphine (P(O-Tol)₃). The base includes an inorganic base, such as cesium carbonate (Cs₂CO₃), sodium carbonate (Na₂CO₃), potassium carbonate (K₂CO₃), potassium fluoride (KF), cesium fluoride (CsF), sodium hydroxide (NaOH), potassium phosphate tribasic (K₃PO₄), sodium tert-butoxide (tert-BuONa) or potassium tert-butoxide (tert-BuOK). The reaction may be carried out in a nonpolar organic solvent such as benzene or toluene, or a polar solvent such as dioxane, tetrahydrofuran, acetonitrile, 1,2-dimethoxyethane or N,N-dimethylformamide. The reaction may be also carried out at a temperature ranging from 50° C. to 150° C., preferably from 80° C. to 110° C. Other reaction conditions, including e.g., reaction time, may be determined from the reaction conditions for conventional Suzuki reaction (Barbara Czako and Laszlo Kurti, STRATEGIC APPLICATIONS of NAMED REACTIONS in ORGANIC SYNTHESIS, 2005).

The compound of Formula 5 is commercially available. The compound of Formula 4 may be prepared according to the following Reaction Schemes 2 or 3.

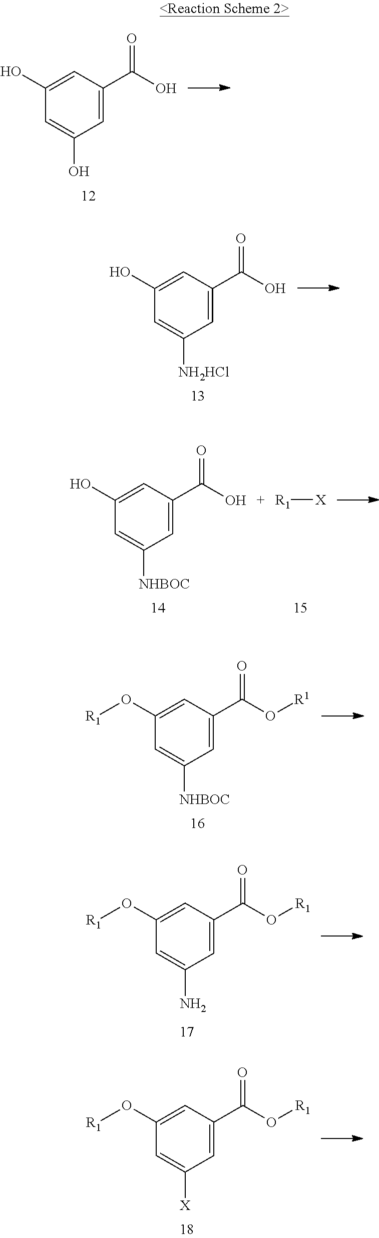

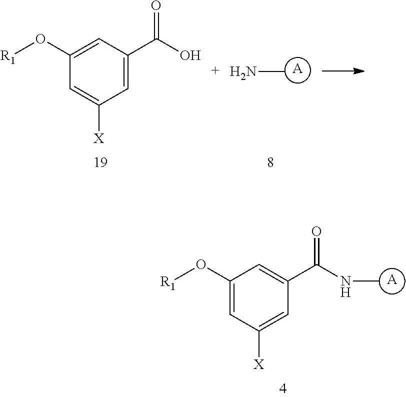

In the Reaction Scheme 2, A and R₁ are the same as defined in the above; X is halogen, O-trifluoromethanesulfonyl, or —OP(O)(OR′)₂; R′ is a C₁-C₆ alkyl group or an aryl group; and BOC is an amine-protecting group.

Specifically, in the Reaction Scheme 2, the compound of Formula 12 may be converted to the compound of Formula 13 via amination reaction. The compound of Formula 12 is commercially available. The amination reaction may be carried out by reacting the compound of Formula 12 with ammonium chloride, ammonium hydroxide, and hydrochloric acid, at a temperature ranging from 100° C. to 180° C.

The compound of Formula 13 may be converted to the compound of Formula 14, by introducing an amine-protecting group thereto, according to conventional methods (Theodora W. Greene and Peter G. M. Wuts, Protective groups in organic synthesis, 3rd Ed., 1999). For example, the introduction of an amine-protecting group may be carried out in a mixed solvent of dioxane and water at room temperature, using an inorganic base such as sodium hydroxide or sodium hydrogen carbonate.

The compound of Formula 14 may be reacted with the commercially available compound of Formula 15 to convert to the compound of Formula 16. The reaction may be carried out according to O-alkylating reaction in the presence of a conventional inorganic base.

A deprotection of the amine-protecting group in the compound of Formula 16 gives the compound of Formula 17. The deprotection may be carried out according to conventional methods (Theodora W. Greene and Peter G. M. Wuts, Protective groups in organic synthesis, 3rd Ed., 1999). For example, the deprotection of the amine-protecting group may be carried out in an organic solvent such as dichloromethane, dioxane or ethyl acetate, at room temperature, using trifluoroacetic acid or hydrochloric acid gas.

The compound of Formula 17 may be converted to the compound of Formula 18 via halogenation. The halogenation is preferably a bromination. For example, the bromination may be carried out using e.g., copper bromide, hydrogen bromide, and sodium nitrite. The bromination may be carried out in a solvent such as water, ethanol, acetonitrile, N,N-dimethylformamide or diethyl ether, at a temperature ranging from 0° C. to 90° C. (Suzuki, Nobutaka; Kaneko, Yoshihiro; Nomoto, Tateo; Izawa, Yasuji, Journal of the Chemical Society, Chemical Communications, 22, 1523-1524, 1984).

The compound of Formula 18 may be converted to the compound of Formula 19 via ester-hydrolysis. The ester-hydrolysis may be carried out using a solution of sodium hydroxide, lithium hydroxide or potassium hydroxide. The reaction may be carried out in water or a mixed solvent of water and a polar solvent such as tetrahydrofuran or ethanol, at a temperature ranging from room temperature to 50° C.

The compound of Formula 19 may be coupled with the compound of Formula 8 to convert to the compound of Formula 4. The coupling reaction may be carried out according to the same methods as in the amide coupling reaction of the Reaction Scheme 1.

And also, the compound of Formula 4 may be prepared according to the following Reaction Scheme 3.

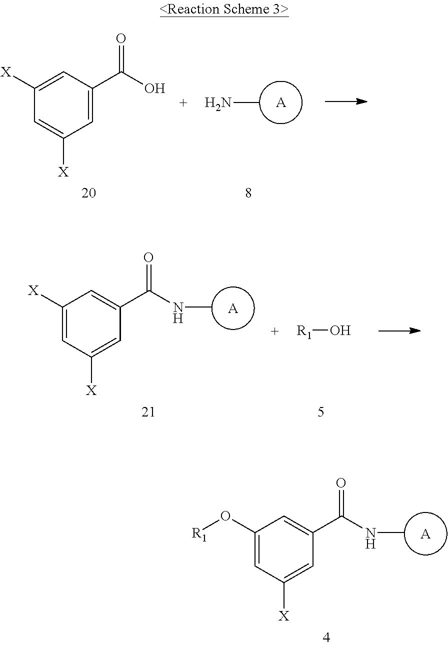

In the Reaction Scheme 3, A and R₁ are the same as defined in the above; X is halogen, O-trifluoromethanesulfonyl, or —OP(O)(OR′)₂; and R′ is a C₁-C₆ alkyl group or an aryl group.

Specifically, the compound of Formula 20 may be coupled with the compound of Formula 8 to convert to the compound of Formula 21. The compounds of Formula 20 and 8 are commercially available. The coupling reaction may be carried out according to the same methods as in the amide coupling reaction of the Reaction Scheme 1.

The compound of Formula 21 may be reacted with the compound of Formula 5 to convert to the compound of Formula 4. The reaction of the compound of Formula 21 and the compound of Formula 5 may be carried out in toluene at about 100° C., in the presence of an inorganic base such as potassium carbonate (Pavia, Michael R.; Taylor, Charles P.; Hershenson, Fred M.; Lobbestael, Sandra J.; Journal of Medicinal Chemistry, 30, 1210-1214, 1987).

The present invention provides a process for preparing a compound of Formula 1b or its pharmaceutically acceptable salt, which comprises reducing a compound of Formula 1a:

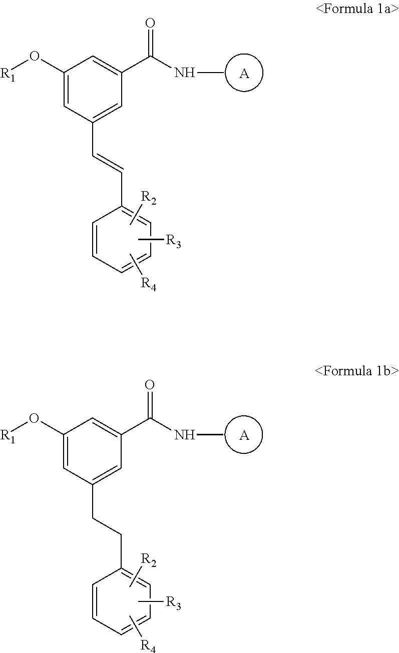

wherein, A, R₁, R₂, R₃, and R₄ are the same as defined in the above.

Specifically, the reduction of the compound of Formula 1a may be carried out in an organic solvent such as ethyl acetate or methanol, using palladium/carbon. Typically, the reduction may be carried out at room temperature using hydrogen gas. The compound of Formula 1a may be prepared according to the Reaction Scheme 1 or 2.

In an embodiment of the present invention, as shown in the following Reaction Scheme 4, there is provided a process for preparing a compound of Formula 1d, which comprises reacting a compound of Formula 1c with R₆—C(O)-halide, R₆—NCO, R₆—NCS, or R₆—SO₂-halide:

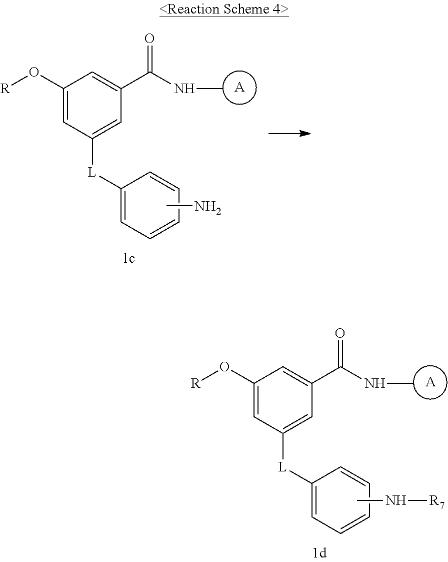

In the Reaction Scheme 4, R₇ is —C(O)—R₆, —C(O)—NH—R₆, —C(S)—NH—R₆, or —SO₂—R₆; and L, A, R₁, and R₆ are the same as defined in the above.

As described in the above, the compound of Formula 1c may be reacted with acyl halides (i.e., R₆—C(O)-halide), isocyanates (i.e., R₆—NCO), isothiocyanates (i.e., R₆—NCS), or sulfonyl halides (i.e., R₆—SO₂-halide), so as to obtain the compound of Formula 1d having amide moiety (R₇═—C(O)—R₆), sulfonamide moiety (R₇═—SO₂—R₆), urea moiety (R₇═—C(O)—NH—R₆), and thiourea moiety (R₇═—C(S)—NH—R₆), respectively. The reaction may be carried out in the presence of an organic base such as triethylamine or diisopropylethylamine or an inorganic base such as potassium carbonate. And also, the reaction may be carried out in a nonpolar solvent such as dichloromethane, tetrahydrofuran or N,N-dimethylformamide, at a temperature ranging from 0° C. to room temperature, for 10 minutes to 12 hours.

The present invention provides a novel intermediate useful for preparing the compound of Formula 1 or its pharmaceutically acceptable salt, i.e., the compound of Formula 2:

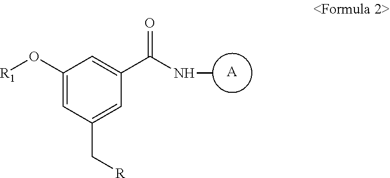

wherein, A, R₁, and R are the same as defined in the above.

The present invention provides a pharmaceutical composition for preventing or treating a glucokinase-mediated disease comprising a therapeutically effective amount of the compound of Formula 1 or its pharmaceutically acceptable salt; and a pharmaceutically acceptable carrier. The glucokinase-mediated disease includes, for example, hyperglycemia, diabetes, insulin resistance in type 2 diabetes, obesity, metabolic syndrome, etc.

The pharmaceutical composition of the present invention may comprise a pharmaceutically acceptable carrier, such as additives, disintegrants, sweeteners, lubricants, or flavoring agents. The pharmaceutical composition may be formulated to an oral dosage form such as tablets, capsules, powders, granules, suspensions, emulsions, or syrups; or a parenteral dosage form such as injection. The dosage form may be various forms, e.g., dosage forms for single administration or for multiple administrations.

And also, the pharmaceutical composition of the present invention may be administered intravenously, intramuscularly, or orally, preferably orally. A typical daily dose of the compound of Formula 1 or its pharmaceutically acceptable salt may range from about 10 mg/kg to about 500 mg/kg (body weight). Of course, the dose may be changed according to the patient's state, age, weight, susceptibility, symptom, or administration route.

The following examples are intended to further illustrate the present invention without limiting its scope of the present invention.

The analyses of the compounds prepared in the following Examples were carried out as follows: Nuclear magnetic resonance (NMR) spectrum analysis was carried out using Bruker 400 MHz spectrometer and chemical shifts thereof were analyzed in ppm. Column chromatography was carried out on silica gel (Merck, 70-230 mesh) (W. C. Still, J. Org. Chem., 43, 2923, 1978).

And also, abbreviations used in the following Examples are as follows: “Me” means methyl. “Et” means ethyl. “Ph” means phenyl. “BOC” means tert-butyloxycarbonyl. “EDAC” means N-(3-dimethylaminopropyl)-N′-ethylcarbodiimide. “HOBT” means 1-hydroxybenzotriazole.

The starting materials in each Example are known compounds, which were synthesized according literatures or obtained from Sigma-Aldrich.

### Preparation 1. 3-bromo-5-isobutoxy-N-(thiazol-2-yl)-benzamide

### Step 1: 3-amino-5-hydroxy-benzoic acid hydrochloride

A mixture of 3,5-dihydroxybenzoic acid (250.0 g), ammonium chloride (213.0 g), and 28% ammonium hydroxide (750.0 mL) was stirred at 180° C. for 3 days under autoclave. The reaction was cooled to room temperature and then concentrated under reduced pressure. The resulting residue was dissolved in a 6 N hydrochloric acid solution (3.0 L). The reaction mixture was heated at 100° C. for 24 hours and then cooled to 70° C.˜80° C. Active carbon (30.0 g) was added to the reaction mixture, which was then filtered with celite pad. The filtrate was concentrated under reduced pressure. The resulting residue was washed with a 6 N hydrochloric acid solution twice and then dried under reduced pressure to obtain 192.0 g of the titled compound (Yield: 62.4%).

¹H-NMR (d₆-DMSO) δ 7.81 (s, 1H), 7.69 (s, 1H), 7.56 (s, 1H)

### Step 2: 3-tert-butoxycarbonylamino-5-hydroxy-benzoic acid

3-Amino-5-hydroxy-benzoic acid hydrochloride (40.0 g) prepared in Step 1, sodium hydroxide (25.3 g), and sodium hydrogen carbonate (21.3 g) were dissolved in distilled water (300.0 mL). (BOC)₂O (46.0 g) and dioxane (200.0 mL) were added to the solution, which was then stirred at room temperature for 12 hours. The reaction mixture was concentrated, acidified with a 2 N hydrochloric acid solution, and then extracted with ethyl acetate three times. The organic layer was dried on anhydrous magnesium sulfate, and then concentrated under reduced pressure to obtain 33.0 g of the titled compound in the form of white foam (Yield: 62.0%).

¹H-NMR (d₆-DMSO) δ 7.34 (s, 1H), 6.95 (s, 1H), 6.72 (s, 1H), 1.24 (s, 9H)

### Step 3: 3-tert-butoxycarbonylamino-5-isobutoxy-benzoic acid isobutyl ester

3-tert-Butoxycarbonylamino-5-hydroxy-benzoic acid (33.0 g) prepared in Step 2, 1-bromo-2-methylpropane (31.2 mL), and potassium carbonate (45.0 g) were added to N,N-dimethylformamide (200.0 mL) and then stirred at 40° C.˜50° C. for 12 hours. The reaction mixture was concentrated and then ethyl acetate was added thereto. The reaction mixture was washed with distilled water and brine three times, dried on anhydrous magnesium sulfate, and then concentrated under reduced pressure to obtain 26.5 g of the titled compound in the form of yellow liquid (Yield: 55.8%).

¹H-NMR (CDCl₃) δ 7.53 (brs, 1H), 7.33 (s, 1H), 7.23 (s, 1H), 6.59 (s, 1H), 4.08 (d, 2H), 3.76 (d, 2H), 2.10-2.02 (m, 2H), 1.52 (s, 9H), 1.03-1.00 (m, 12H)

### Step 4: 3-amino-5-isobutoxy-benzoic acid isobutyl ester

3-tert-Butoxycarbonylamino-5-isobutoxy-benzoic acid isobutyl ester (26.5 g) prepared in Step 3 was dissolved in dichloromethane (300.0 mL). Trifluoroacetic acid (30.0 mL) was added to the solution, which was then stirred at room temperature for 12 hours. The reaction mixture was concentrated and then ethyl acetate was added thereto. The reaction mixture was washed with a saturated sodium hydrogen carbonate solution and brine three times, dried on anhydrous magnesium sulfate, and then concentrated under reduced pressure to obtain 21.0 g of the titled compound in the form of yellow liquid (Yield: 99.0%).

¹H-NMR (CDCl₃) δ 6.98-6.96 (m, 2H), 6.41 (t, 1H), 4.07 (d, 2H), 3.72 (d, 2H), 3.65 (brs, 2H), 2.10-2.02 (m, 2H), 1.03-0.98 (m, 12H)

### Step 5: 3-bromo-5-isobutoxy-benzoic acid isobutyl ester

3-Amino-5-isobutoxy-benzoic acid isobutyl ester (21.0 g) prepared in Step 4 was dissolved in 48% hydrogen bromide (30.0 mL) and ethanol (200.0 mL). A solution of sodium nitrite (6.6 g) in distilled water (60.0 mL) was slowly added to the solution at 0° C. A solution of copper bromide (5.7 g) in 48% hydrogen bromide (150.0 mL) was added to the reaction mixture, which was then stirred at 80° C.˜90° C. for 20 minutes. The reaction mixture was concentrated under reduced pressure and then extracted with ethyl acetate. The organic layer was washed with distilled water, a saturated sodium hydrogen carbonate solution, and brine three times, dried on anhydrous magnesium sulfate, and then concentrated under reduced pressure to obtain a residue in the form of yellow liquid. The residue was purified with silica gel column chromatography (eluent: n-hexane/ethyl acetate=5/1) to obtain 12.0 g of the titled compound in the form of yellow liquid (Yield: 46.1%).

¹H-NMR (CDCl₃) δ 7.72 (d, 1H), 7.48 (d, 1H), 7.23 (t, 1H), 4.10 (d, 2H), 3.74 (d, 2H), 2.12-2.05 (m, 2H), 1.04-1.01 (m, 12H)

### Step 6: 3-bromo-5-isobutoxy-benzoic acid

3-Bromo-5-isobutoxy-benzoic acid isobutyl ester (12.0 g) prepared in Step 5 was dissolved in methanol (100.0 mL). 10% sodium hydroxide solution (100.0 mL) was added to the solution, which was then stirred at 50° C. for 2 hours. The reaction mixture was concentrated under reduced pressure, acidified with a 3 N hydrochloric acid solution, and then extracted with ethyl acetate. The organic layer was washed with brine, dried on anhydrous magnesium sulfate, and then concentrated under reduced pressure to 9.5 g of the titled compound in the form of white solid (Yield: 95.4%).

¹H-NMR (CDCl₃) δ 7.81 (t, 1H), 7.54 (dd, 1H), 7.29 (t, 1H), 3.76 (d, 2H), 2.15-2.05 (m, 1H), 1.04 (d, 6H)

### Step 7: 3-bromo-5-isobutoxy-N-(thiazol-2-yl)-benzamide

3-Bromo-5-isobutoxy-benzoic acid (5.0 g) prepared in Step 6 was dissolved in thionyl chloride (20.0 mL). The reaction mixture was heated at 100° C. for 2 hours and then cooled to room temperature. The reaction mixture was concentrated under reduced pressure and then dissolved in dichloromethane (150.0 mL). Triethylamine (3.8 mL) and 2-aminothiazole (1.8 g) were added to the resulting solution, which was then stirred at room temperature for 12 hours. The reaction mixture was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=6/1) to obtain 5.0 g of the titled compound in the form of white foam (Yield: 76.9%).

¹H-NMR (CDCl₃) δ 12.10 (brs, 1H), 7.66 (t, 1H), 7.44 (dd, 1H), 7.29 (dd, 1H), 7.19 (d, 1H), 7.00 (d, 1H), 3.74 (d, 2H), 2.12-2.06 (m, 1H), 1.02 (d, 6H)

### Preparation 2. 3-(triphenylphosphonium bromide-methyl)-5-isobutoxy-N-(thiazol-2-yl)-benzamide

### Step 1: 5-isobutoxy-isophthalic acid dimethyl ester

Dimethyl 5-hydroxyisophthalate (5.0 g), 1-bromo-2-methylpropane (3.1 mL), and potassium carbonate (4.9 g) were dissolved in N,N-dimethylformamide (100.0 mL). The reaction mixture was heated at 40° C. for 20 hours and then cooled to room temperature. The reaction mixture was concentrated and ethyl acetate was added thereto. The reaction mixture was washed with distilled water and brine three times, dried on anhydrous magnesium sulfate, and then concentrated under reduced pressure to obtain 6.3 g of the titled compound in the form of yellow liquid (Yield: 99.5%).

¹H-NMR (CDCl₃) δ 8.26 (t, 1H), 7.74 (d, 2H), 3.94 (s, 6H), 3.80 (d, 2H), 2.15-2.04 (m, 1H), 1.04 (d, 6H)

### Step 2: 5-isobutoxy-isophthalic acid monomethyl ester

5-Isobutoxy-isophthalic acid dimethyl ester (6.3 g) prepared in Step 1 was dissolved in tetrahydrofuran (100.0 mL). A solution of 1 N potassium hydroxide in methanol (21.0 mL) was added to the solution, which was then stirred at 100° C. for 10 hours. The reaction mixture was concentrated and then ethyl acetate was added thereto. The reaction mixture was washed with 2 N hydrochloric acid solution, distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=3/1) to obtain 3.4 g of the titled compound in the form of white solid (Yield: 57.0%).

¹H-NMR (CDCl₃) δ 8.35 (t, 1H), 7.80 (d, 2H), 3.96 (s, 3H), 3.83 (d, 2H), 2.15-2.10 (m, 1H), 1.06 (d, 6H)

### Step 3: 5-isobutoxy-N-(thiazol-2-yl)-isophthalic acid methyl ester

5-Isobutoxy-isophthalic acid monomethyl ester (3.4 g) prepared in Step 2 and 2-aminothiazole (1.5 g) were dissolved in dichloromethane (100.0 mL). DCC (3.3 g) was added to the solution, which was then stirred at room temperature for 12 hours. The reaction mixture was concentrated and then purified with silica gel column chromatography (eluent: n-hexane/ethyl acetate=3/1) to obtain 2.3 g of the titled compound in the form of white solid (Yield: 51.0%).

¹H-NMR (CDCl₃) δ 8.20 (s, 1H), 7.81-7.79 (m, 1H), 7.76-7.74 (m, 1H), 7.14 (d, 1H), 6.97 (d, 1H), 3.96 (s, 3H), 3.83 (d, 2H), 2.15-2.10 (m, 1H), 1.06 (d, 6H)

### Step 4: 3-hydroxymethyl-5-isobutoxy-N-(thiazol-2-yl)-benzamide

5-Isobutoxy-N-(thiazol-2-yl)-isophthalic acid methyl ester (1.8 g) prepared in Step 3 was dissolved in ethanol (10.0 mL) and tetrahydrofuran (50.0 mL). A solution of 2 N lithium borohydride in tetrahydrofuran (10.0 mL) was slowly added at 0° C. to the solution, which was then stirred at room temperature for 2 days. The reaction mixture was concentrated, acidified with 10% citric acid, and then extracted with ethyl acetate. The organic layer was washed with brine, dried on anhydrous magnesium sulfate, and then concentrated under reduced pressure to obtain a residue in the form of yellow liquid. The residue was purified with silica gel column chromatography (eluent: n-hexane/ethyl acetate=5/1) to obtain 1.4 g of the titled compound in the form of white solid (Yield: 84.6%).

¹H-NMR (CDCl₃) δ 7.51 (s, 1H), 7.43 (s, 1H), 7.16-7.14 (m, 2H), 6.95 (d, 1H), 4.70 (s, 2H), 3.73 (d, 2H), 2.12-2.04 (m, 1H), 1.03 (d, 6H)

### Step 5: 3-bromomethyl-5-isobutoxy-N-(thiazol-2-yl)-benzamide

3-Hydroxymethyl-5-isobutoxy-N-(thiazol-2-yl)-benzamide (1.4 g) prepared in Step 4 was dissolved in tetrahydrofuran (50.0 mL). Tribromo phosphate (1.5 g) was slowly added at 0° C. to the solution, which was then stirred at room temperature for 12 hours. The reaction mixture was concentrated and then ethyl acetate was added thereto. The reaction mixture was washed with a saturated sodium hydrogen carbonate solution and brine three times, dried on anhydrous magnesium sulfate, and then concentrated under reduced pressure to obtain residue in the form of yellow liquid. The residue was purified with silica gel column chromatography (eluent: n-hexane/ethyl acetate=3/1) to obtain 0.7 g of the titled compound in the form of white solid (Yield: 41.5%).

¹H-NMR (CDCl₃) δ 7.56 (s, 1H), 7.48-7.47 (m, 1H), 7.19 (t, 1H), 7.13 (d, 1H), 6.98 (d, 1H), 4.68 (s, 2H), 3.73 (d, 2H), 2.12-2.04 (m, 1H), 1.04 (d, 6H)

### Step 6: 3-(triphenylphosphonium bromide-methyl)-5-isobutoxy-N-(thiazol-2-yl)-benzamide

3-Bromomethyl-5-isobutoxy-N-(thiazol-2-yl)-benzamide (0.7 g) prepared in Step 5 and triphenylphosphine (0.6 g) were dissolved in benzene (50.0 mL). The reaction mixture was heated at 100° C. for 6 hours and then cooled to room temperature. The reaction mixture was concentrated and then washed with diethyl ether three times to obtain 1.1 g of the titled compound in the form of white solid (Yield: 91.5%).

¹H-NMR (CDCl₃) δ 12.76 (brs, 1H), 8.10-8.05 (m, 2H), 7.95-7.84 (m, 15H), 7.72 (d, 1H), 7.53 (s, 1H), 7.44 (d, 1H), 5.39 (d, 2H), 3.71 (d, 2H), 2.10-2.01 (m, 1H), 1.07 (d, 6H)

### Preparation 3. 3-[(phosphonic acid diethyl ester)-methyl]-5-(2-methoxy-(1S)-methyl-ethoxy)-N-(thiazol-2-yl)-benzamide

### Step 1: 5-(2-methoxy-(1S)-methyl-ethoxy)-isophthalic acid dimethyl ester

Dimethyl 5-hydroxyisophthalate (10.7 g), (R)-(−)-1-methoxy-2-propanol (5.0 mL), and triphenylphosphine (16.1 g) were dissolved in tetrahydrofuran (300.0 mL). Diiso-propylazodicarboxylate (12.0 mL) was slowly added at 0° C. to the solution, which was then stirred at room temperature for 12 hours. The reaction mixture was concentrated and then purified with silica gel column chromatography (eluent: n-hexane/ethyl acetate=5/1) to obtain 13.6 g of the titled compound in the form of yellow liquid (Yield: 94.5%).

¹H-NMR (CDCl₃) δ 8.27 (t, 1H), 7.79 (d, 2H), 4.69-4.64 (m, 1H), 3.93 (s, 6H), 3.63-3.50 (m, 2H), 3.42 (s, 3H), 1.32 (d, 3H)

### Step 2: 5-(2-methoxy-(1S)-methyl-ethoxy)-isophthalic acid monomethyl ester

5-(2-Methoxy-(1S)-methyl-ethoxy)-isophthalic acid dimethyl ester (13.6 g) prepared in Step 1 was dissolved in methanol (500.0 mL). Potassium hydroxide (2.4 g) was added to the solution, which was then stirred at 100° C. for 12 hours. The reaction mixture was concentrated and then distilled water was added thereto. The reaction mixture was washed with diethyl ether three times, acidified with 1 N hydrochloric acid solution, and then extracted with ethyl acetate. The organic layer was dried on anhydrous magnesium sulfate and then concentrated under reduced pressure to obtain 10.1 g of the titled compound in the form of yellow liquid (Yield: 78.4%).

¹H-NMR (CDCl₃) δ 8.34 (t, 1H), 7.85-7.83 (m, 2H), 4.73-4.65 (m, 1H), 3.95 (s, 3H), 3.65-3.53 (m, 2H), 3.44 (s, 3H), 1.35 (d, 3H)

### Step 3: 5-(2-methoxy-(1S)-methyl-ethoxy)-N-(thiazol-2-yl)-isophthalic acid methyl ester

5-(2-Methoxy-(1S)-methyl-ethoxy)-isophthalic acid monomethyl ester (5.0 g) prepared in Step 2 was dissolved in thionyl chloride (50.0 mL). The reaction mixture was heated at 100° C. for 2 hours and then cooled to room temperature. The reaction mixture was concentrated. The resulting residue was dissolved in dichloromethane (100.0 mL). Triethylamine (3.9 mL) and 2-aminothiazole (2.2 g) were added to the solution, which was then stirred at room temperature for 12 hours. The reaction mixture was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/1) to obtain 4.7 g of the titled compound in the form of white foam (Yield: 72.0%).

¹H-NMR (CDCl₃) δ 11.92 (brs, 1H), 8.19 (t, 1H), 7.84 (dd, 1H), 7.79 (t, 1H), 7.19 (d, 1H), 6.97 (d, 1H), 4.70-4.65 (m, 1H), 3.90 (s, 3H), 3.63-3.50 (m, 2H), 3.41 (s, 3H), 1.34 (d, 3H)

### Step 4

### 3-hydroxymethyl-5-(2-methoxy-(1S)-methyl-ethoxy)-N-(thiazol-2-yl)-benzamide

5-(2-Methoxy-(1S)-methyl-ethoxy)-N-(thiazol-2-yl)-isophthalic acid methyl ester (4.7 g) prepared in Step 3 was dissolved in a mixed solvent of ethanol (25.0 mL) and tetrahydrofuran (250.0 mL). A solution of 2 N lithium borohydride in tetrahydrofuran (50.0 mL) was slowly added at 0° C. to the solution, which was then stirred at room temperature for 12 hours. The reaction mixture was concentrated, acidified with 10% citric acid solution, and then extracted with ethyl acetate. The organic layer was washed with distilled water and brine three times, dried on anhydrous magnesium sulfate, and then concentrated under reduced pressure to obtain 2.7 g of the titled compound in the form of white foam (Yield: 62.5%).

¹H-NMR (CDCl₃) δ 10.40 (brs, 1H), 7.52-7.44 (m, 2H), 7.28 (s, 1H), 7.15 (d, 1H), 6.94 (d, 1H), 4.75 (s, 2H), 4.65-4.60 (m, 1H), 3.60-3.50 (m, 2H), 3.42 (s, 3H), 1.28 (d, 3H)

### Step 5

### 3-bromomethyl-5-(2-methoxy-(1S)-methyl-ethoxy)-N-(thiazol-2-yl)-benzamide

3-Hydroxymethyl-5-(2-methoxy-(1S)-methyl-ethoxy)-N-(thiazol-2-yl)-benzamide (2.7 g) prepared in Step 4 was dissolved in tetrahydrofuran (50.0 mL). Tribromo phosphate (1.0 mL) was added to the solution at 0° C. The reaction mixture was stirred at room temperature for 12 hours and then concentrated. Ethyl acetate was added to the resulting residue. The reaction mixture was washed with a saturated sodium hydrogen carbonate solution, distilled water, and brine three times, dried on anhydrous magnesium sulfate, and then concentrated under reduced pressure to 1.5 g of the titled compound in the form of white solid (Yield: 45.8%).

¹H-NMR (CDCl₃) δ 12.42 (brs, 1H), 7.53 (d, 2H), 7.22 (s, 1H), 7.14 (d, 1H), 6.98 (d, 1H), 4.65-4.59 (m, 1H), 4.43 (s, 2H), 3.61-3.48 (m, 2H), 3.41 (s, 3H), 1.33 (d, 3H)

### Step 6: 3-[(phosphonic acid diethyl ester)-methyl]-5-(2-methoxy-(1S)-methyl-ethoxy)-N-(thiazol-2-yl)-benzamide

3-Bromomethyl-5-(2-methoxy-(1S)-methyl-ethoxy)-N-(thiazol-2-yl)-benzamide (1.5 g) prepared in Step 5 was dissolved in triethyl phosphite (3.5 mL). The resulting solution was stirred at 160° C. for 3 hours and then concentrated under reduced pressure. The resulting residue was purified with silica gel column chromatography (eluent: dichloromethane/methanol=20/1) to obtain 1.5 g of the titled compound in the form of yellow liquid (Yield: 88.3%).

¹H-NMR (CDCl₃) δ 11.78 (brs, 1H), 7.46 (dd, 2H), 7.25 (t, 1H), 7.13 (d, 1H), 6.97 (d, 1H), 4.64-4.58 (m, 1H), 4.10-4.02 (m, 4H), 3.59-3.45 (m, 2H), 3.40 (s, 3H), 3.15 (d, 2H), 1.33 (d, 3H), 1.28-1.24 (m, 6H)

### Preparation 4. 3-[(phosphonic acid diethyl ester)-methyl]-5-(2-methoxy-(1S)-methyl-ethoxy)-N-(5-fluorothiazol-2-yl)-benzamide

### Step 1: 3-hydroxymethyl-5-(2-methoxy-(1S)-methyl-ethoxy)-benzoic acid methyl ester

5-(2-Methoxy-(1S)-methyl-ethoxy)-isophthalic acid monomethyl ester (30.0 g) prepared in Step 2 of Preparation 3 was dissolved in tetrahydrofuran (600.0 mL). A solution of 1 M tetrahydrofuran-borane complex in tetrahydrofuran (300.0 mL) was slowly added to the solution at 0° C. The reaction mixture was stirred at room temperature for 12 hours. Distilled water (900.0 mL) was slowly added at 0° C. to the reaction mixture, which was then stirred for 3 hours. The reaction mixture was concentrated under reduced pressure and then extracted with ethyl acetate. The organic layer was washed with distilled water and brine three times, dried on anhydrous magnesium sulfate, and then concentrated under reduced pressure to obtain 26.3 g of the titled compound in the form of yellow liquid (Yield: 92.5%).

¹H-NMR (CDCl₃) δ 7.61 (s, 1H), 7.51 (t, 1H), 7.16 (s, 1H), 4.69 (s, 2H), 4.65-4.60 (m, 1H), 3.90 (s, 3H), 3.61-3.48 (m, 2H), 3.41 (s, 3H), 1.31 (d, 3H)

### Step 2: 3-hydroxymethyl-5-(2-methoxy-(1S)-methyl-ethoxy)-benzoic acid

3-Hydroxymethyl-5-(2-methoxy-(1S)-methyl-ethoxy)-benzoic acid methyl ester (20.0 g) prepared in Step 1 was dissolved in tetrahydrofuran (300.0 mL). A 3 N sodium hydroxide solution (300.0 mL) was added to the solution, which was then stirred at room temperature for 12 hours. The reaction mixture was concentrated, acidified with 1 N hydrochloric acid solution, and then extracted with ethyl acetate. The organic layer was dried on anhydrous magnesium sulfate, and then concentrated under reduced pressure to obtain 18.9 g of the titled compound in the form of yellow liquid (Yield: 99.9%).

¹H-NMR (CDCl₃) δ 7.66 (s, 1H), 7.56 (s, 1H), 7.21 (s, 1H), 4.71 (s, 2H), 4.66-4.62 (m, 1H), 3.63-3.50 (m, 2H), 3.42 (s, 3H), 1.32 (d, 3H)

### Step 3: 3-acetoxymethyl-5-(2-methoxy-(1S)-methyl-ethoxy)-benzoic acid

3-Hydroxymethyl-5-(2-methoxy-(1S)-methyl-ethoxy)-benzoic acid (18.9 g) prepared in Step 2 was dissolved in dichloromethane (500.0 mL). Pyridine (26.0 mL) and acetyl chloride (11.2 mL) were added at 0° C. to the solution, which was then stirred at room temperature for 2 hours. Distilled water (500.0 mL) was added to the reaction mixture, which was then stirred at room temperature for 12 hours. The reaction mixture was washed with 1 N hydrochloric acid solution, dried on anhydrous magnesium sulfate, and then concentrated under reduced pressure to obtain 21.9 g of the titled compound in the form of yellow liquid (Yield: 98.6%).

¹H-NMR (CDCl₃) δ 7.68 (s, 1H), 7.61 (d, 1H), 7.18 (s, 1H), 5.11 (s, 2H), 4.67-4.62 (m, 1H), 3.63-3.51 (m, 2H), 3.43 (s, 3H), 2.13 (s, 3H), 1.34 (d, 3H)

### Step 4

### 3-acetoxymethyl-5-(2-methoxy-(1S)-methyl-ethoxy)-N-(5-fluorothiazol-2-yl)-benzamide

3-Acetoxymethyl-5-(2-methoxy-(1S)-methyl-ethoxy)-benzoic acid (2.0 g) prepared in Step 3 was dissolved in thionyl chloride (20.0 mL). The solution was stirred at 100° C. for 3 hours and then concentrated. The resulting residue was dissolved in pyridine (50.0 mL). 2-Amino-5-fluorothiazole hydrochloride (1.0 g) was added to the solution, which was then stirred at room temperature for 12 hours. The reaction mixture was concentrated and then dichloromethane was added thereto. The reaction mixture was washed with 1 N hydrochloric acid solution, a saturated sodium hydrogen carbonate solution, distilled water, and brine three times, dried on anhydrous magnesium sulfate, and then concentrated under reduced pressure to obtain a residue in the form of yellow liquid. The resulting residue was purified with silica gel column chromatography (eluent: n-hexane/ethyl acetate=1/2) to obtain 0.9 g of the titled compound in the form of pale yellow liquid (Yield: 33.1%).

¹H-NMR (CDCl₃) δ 12.28 (brs, 1H), 7.49-7.47 (m, 2H), 7.19 (s, 1H), 6.64 (d, 1H), 5.10 (s, 2H), 4.65-4.61 (m, 1H), 3.62-3.49 (m, 2H), 3.41 (s, 3H), 2.10 (s, 3H), 1.33 (d, 3H)

### Step 5

### 3-hydroxymethyl-5-(2-methoxy-(1S)-methyl-ethoxy)-N-(5-fluorothiazol-2-yl)-benzamide

3-Acetoxymethyl-5-(2-methoxy-(1S)-methyl-ethoxy)-N-(5-fluorothiazol-2-yl)-benza mide (0.9 g) prepared in Step 4 was dissolved in tetrahydrofuran (10.0 mL). A 3 N sodium hydroxide solution (10.0 mL) was added to the solution, which was then stirred at room temperature for 12 hours. The reaction mixture was concentrated, and then extracted with ethyl acetate three times. The organic layer was dried on anhydrous magnesium sulfate and then concentrated under reduced pressure to obtain 0.8 g of the titled compound in the form of white solid (Yield: 99.9%).

¹H-NMR (CDCl₃) δ 10.45 (brs, 1H), 7.46 (s, 1H), 7.41 (d, 1H), 7.18 (s, 1H), 6.92 (d, 1H), 4.73 (s, 2H), 4.66-4.62 (m, 1H), 3.62-3.49 (m, 2H), 3.41 (s, 3H), 1.33 (d, 3H)

### Step 6

### 3-bromomethyl-5-(2-methoxy-(1S)-methyl-ethoxy)-N-(5-fluorothiazol-2-yl)-benzamide

3-Hydroxymethyl-5-(2-methoxy-(1S)-methyl-ethoxy)-N-(5-fluorothiazol-2-yl)-benza mide (0.8 g) prepared in Step 5 was dissolved in tetrahydrofuran (10.0 mL). Tribromo phosphate (0.3 mL) was added at 0° C. to the solution, which was then stirred at room temperature for 12 hours. The reaction mixture was concentrated and then ethyl acetate was added thereto. The reaction mixture was washed with a saturated sodium hydrogen carbonate solution, distilled water and brine three times, dried on anhydrous magnesium sulfate, and then concentrated under reduced pressure to obtain 0.5 g of the titled compound in the form of white foam (Yield: 49.6%).

¹H-NMR (CDCl₃) δ 12.17 (brs, 1H), 7.50 (t, 1H), 7.44 (t, 1H), 7.23 (t, 1H), 6.69 (d, 1H), 4.65-4.61 (m, 1H), 4.44 (s, 2H), 3.62-3.49 (m, 2H), 3.41 (s, 3H), 1.33 (d, 3H)

### Step 7: 3-[(phosphonic acid diethyl ester)-methyl]-5-(2-methoxy-(1S)-methyl-ethoxy)-N-(5-fluorothiazol-2-yl)-benzamide

3-Bromomethyl-5-(2-methoxy-(1S)-methyl-ethoxy)-N-(5-fluorothiazol-2-yl)-benzam ide (0.5 g) prepared in Step 6 was dissolved in triethyl phosphite (1.0 mL). The reaction mixture was heated at 160° C. for 3 hours and then cooled to room temperature. The reaction mixture was concentrated under reduced pressure and then purified with silica gel column chromatography (eluent: dichloromethane/methanol=20/1) to obtain 0.6 g of the titled compound in the form of yellow liquid (Yield: 99.9%).

¹H-NMR (CDCl₃) δ 10.53 (brs, 1H), 7.40 (t, 2H), 7.13 (d, 1H), 6.93 (d, 1H), 4.66-4.58 (m, 1H), 4.15-4.00 (m, 4H), 3.60-3.48 (m, 2H), 3.40 (s, 3H), 3.15 (d, 2H), 1.32 (d, 3H), 1.29-1.24 (m, 6H)

### Preparation 5. 3-[(phosphonic acid diethyl ester)-methyl]-5-(2-methoxy-(1S)-methyl-ethoxy)-N-(1-methyl-1H-pyrazol-3-yl)-benz amide

### Step 1: 3-bromomethyl-5-(2-methoxy-(1S)-methyl-ethoxy)-benzoic acid methyl ester

3-Hydroxymethyl-5-(2-methoxy-(1S)-methyl-ethoxy)-benzoic acid methyl ester (6.3 g) prepared in Step 1 of Preparation 4 was dissolved in tetrahydrofuran (300.0 mL). Tribromo phosphate (2.6 mL) was added at 0° C. to the solution, which was then stirred at room temperature for 12 hours. The reaction mixture was concentrated and then ethyl acetate was added thereto. The reaction mixture was stirred with a saturated sodium hydrogen carbonate solution, distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=2/1) to obtain 5.2 g of the titled compound in the form of pale yellow liquid (Yield: 66.1%).

¹H-NMR (CDCl₃) δ 7.64 (t, 1H), 7.52 (dd, 1H), 7.16 (t, 1H), 4.65-4.59 (m, 1H), 4.42 (s, 2H), 3.91 (s, 3H), 3.61-3.48 (m, 2H), 3.42 (s, 3H), 1.32 (d, 3H)

### Step 2: 3-[(phosphonic acid diethyl ester)-methyl]-5-(2-methoxy-(1S)-methyl-ethoxy)-benzoic acid methyl ester

3-Bromomethyl-5-(2-methoxy-(1S)-methyl-ethoxy)-benzoic acid methyl ester (5.2 g) prepared in Step 1 was dissolved in triethyl phosphite (14.3 mL). The reaction mixture was heated at 160° C. for 3 hours and then cooled to room temperature. The reaction mixture was concentrated under reduced pressure and then purified with silica gel column chromatography (eluent: dichloromethane/methanol=20/1) to obtain 6.1 g of the titled compound in the form of yellow liquid (Yield: 99.2%).

¹H-NMR (CDCl₃) δ 7.54 (d, 1H), 7.49 (d, 1H), 7.10 (d, 1H), 4.63-4.58 (m, 1H), 4.14-4.00 (m, 4H), 3.89 (s, 3H), 3.60-3.47 (m, 2H), 3.41 (s, 3H), 3.14 (d, 2H), 1.31 (d, 3H), 1.28-1.24 (m, 6H)

### Step 3: 3-[(phosphonic acid diethyl ester)-methyl]-5-(2-methoxy-(1S)-methyl-ethoxy)-benzoic acid

3-[(Phosphonic acid diethyl ester)-methyl]-5-(2-methoxy-(1S)-methyl-ethoxy)-benzoic acid methyl ester (5.1 g) prepared in Step 2 was dissolved in tetrahydrofuran (50.0 mL). A 3 N sodium hydroxide solution (50.0 mL) was added to the solution, which was then stirred at room temperature for 12 hours. The reaction mixture was concentrated, acidified with 1 N hydrochloric acid solution, and then extracted with ethyl acetate. The organic layer was dried on anhydrous magnesium sulfate and then concentrated under reduced pressure to obtain 5.0 g of the titled compound in the form of yellow liquid (Yield: 93.6%).

¹H-NMR (CDCl₃) δ 9.07 (brs, 1H), 7.62 (t, 1H), 7.51 (dd, 1H), 7.09 (dd, 1H), 4.63-4.55 (m, 1H), 4.18-4.00 (m, 4H), 3.61-3.48 (m, 2H), 3.41 (s, 3H), 3.18 (d, 2H), 1.30 (d, 3H), 1.29-1.24 (m, 6H)

### Step 4: 3-[(phosphonic acid diethyl ester)-methyl]-5-(2-methoxy-(1S)-methyl-ethoxy)-N-(1-methyl-1H-pyrazol-3-yl)-benz amide

3-[(Phosphonic acid diethyl ester)-methyl]-5-(2-methoxy-(1S)-methyl-ethoxy)-benzoic acid (3.0 g) prepared in Step 3, HOBT (2.3 g), EDAC (3.2 g), triethylamine (2.3 mL) and 1-methyl-1H-pyrazol-3-ylamine (0.8 g) were added to dichloromethane (100.0 mL). The reaction mixture was stirred at room temperature for 12 hours. The reaction mixture was washed with a 1 N hydrochloric acid solution, a saturated sodium hydrogen carbonate solution, distilled water and brine three times, dried on anhydrous magnesium sulfate, and then concentrated under reduced pressure to obtain a residue in the form of yellow liquid. The residue was purified with silica gel column chromatography (eluent: dichloromethane/methanol=20/1) to obtain 1.5 g of the titled compound in the form of yellow liquid (Yield: 41.8%).

¹H-NMR (CDCl₃) δ 8.71 (brs, 1H), 7.36 (d, 1H), 7.34 (d, 1H), 7.29 (d, 1H), 7.07 (d, 1H), 6.80 (d, 1H), 4.66-4.58 (m, 1H), 4.10-4.00 (m, 4H), 3.80 (s, 3H), 3.60-3.48 (m, 2H), 3.40 (s, 3H), 3.15 (d, 2H), 1.33 (d, 3H), 1.27-1.24 (m, 6H)

### Preparation 6. 3-[(phosphonic acid diethyl ester)-methyl]-5-(2-methoxy-(1S)-methyl-ethoxy)-N-(pyrazin-2-yl)-benzamide

### Step 1

### 3-acetoxymethyl-5-(2-methoxy-(1S)-methyl-ethoxy)-N-(pyrazin-2-yl)-benzamide

3-Acetoxymethyl-5-(2-methoxy-(1S)-methyl-ethoxy)-benzoic acid (5.0 g) prepared in Step 3 of Preparation 4 was dissolved in thionyl chloride (30.0 mL). The reaction mixture was heated at 100° C. for 3 hours and then cooled to room temperature. The reaction mixture was concentrated. The resulting residue was dissolved in pyridine (50.0 mL). 2-Aminopyrazine (1.7 g) was added to the solution, which was stirred at room temperature for 12 hours. The reaction mixture was concentrated and then dichloromethane was added thereto. The reaction mixture was washed with a 1 N hydrochloric acid solution, a saturated sodium hydrogen carbonate solution, distilled water and brine three times, dried on anhydrous magnesium sulfate, and then concentrated under reduced pressure to obtain a residue in the form of yellow liquid. The residue was purified with silica gel column chromatography (eluent: dichloromethane/methanol=20/1) to obtain 6.0 g of the titled compound in the form of pale yellow liquid (Yield: 94.3%).

¹H-NMR (CDCl₃) δ 9.70 (d, 1H), 8.46 (brs, 1H), 8.40 (d, 1H), 8.29 (dd, 1H), 7.48-7.45 (m, 2H), 7.16 (d, 1H), 5.13 (s, 2H), 4.70-4.62 (m, 1H), 3.63-3.51 (m, 2H), 3.43 (s, 3H), 2.14 (s, 3H), 1.35 (d, 3H)

### Step 2

### 3-hydroxymethyl-5-(2-methoxy-(1S)-methyl-ethoxy)-N-(pyrazin-2-yl)-benzamide

3-Acetoxymethyl-5-(2-methoxy-(1S)-methyl-ethoxy)-N-(pyrazin-2-yl)-benzamide (6.0 g) prepared in Step 1 was dissolved in tetrahydrofuran (50.0 mL). A 3 N sodium hydroxide solution (50.0 mL) was added to the solution, which was then stirred at room temperature for 12 hours. The reaction mixture was concentrated and then extracted with ethyl acetate three times. The organic layer was dried on anhydrous magnesium sulfate and then concentrated under reduced pressure to obtain 4.0 g of the titled compound in the form of yellow liquid (Yield: 75.5%).

¹H-NMR (CDCl₃) δ 9.69 (d, 1H), 8.58 (brs, 1H), 8.39 (d, 1H), 8.29 (dd, 1H), 7.48 (s, 1H), 7.44 (t, 1H), 7.17 (s, 1H), 4.75 (s, 2H), 4.69-4.64 (m, 1H), 3.63-3.51 (m, 2H), 3.42 (s, 3H), 1.34 (d, 3H)

### Step 3

### 3-bromomethyl-5-(2-methoxy-(1S)-methyl-ethoxy)-N-(pyrazin-2-yl)-benzamide

3-Hydroxymethyl-5-(2-methoxy-(1S)-methyl-ethoxy)-N-(pyrazin-2-yl)-benzamide (4.0 g) prepared in Step 2 was dissolved in tetrahydrofuran (50.0 mL). Tribromo phosphate (1.4 mL) was slowly added at 0° C. to the solution, which was then stirred at room temperature for 12 hours. The reaction mixture was concentrated and then ethyl acetate was added thereto. The reaction mixture was washed with a saturated sodium hydrogen carbonate solution, distilled water and brine three times, dried on anhydrous magnesium sulfate, and then concentrated under reduced pressure to obtain 4.2 g of the titled compound in the form of yellow liquid (Yield: 86.8%).

¹H-NMR (CDCl₃) δ 9.69 (d, 1H), 8.45 (brs, 1H), 8.40 (d, 1H), 8.29 (dd, 1H), 7.49 (s, 1H), 7.44 (t, 1H), 7.19 (t, 1H), 4.69-4.64 (m, 1H), 4.49 (s, 2H), 3.63-3.51 (m, 2H), 3.42 (s, 3H), 1.35 (d, 3H)

### Step 4: 3-[(phosphonic acid diethyl ester)-methyl]-5-(2-methoxy-(1S)-methyl-ethoxy)-N-(pyrazin-2-yl)-benzamide

3-Bromomethyl-5-(2-methoxy-(1S)-methyl-ethoxy)-N-(pyrazin-2-yl)-benzamide (4.2 g) prepared in Step 3 was dissolved in triethyl phosphite (9.5 mL). The reaction mixture was heated at 160° C. for 3 hours and then cooled to room temperature. The reaction mixture was concentrated under reduced pressure and then purified with silica gel column chromatography (eluent: dichloromethane/methanol=20/1) to obtain 3.2 g of the titled compound in the form of yellow liquid (Yield: 66.0%).

¹H-NMR (CDCl₃) δ 9.68 (d, 1H), 8.77 (brs, 1H), 8.38 (d, 1H), 8.28 (dd, 1H), 7.42 (d, 2H), 7.11 (d, 1H), 4.69-4.62 (m, 1H), 4.15-4.00 (m, 4H), 3.62-3.48 (m, 2H), 3.42 (s, 3H), 3.18 (d, 2H), 1.34 (d, 3H), 1.30-1.24 (m, 6H)

### Preparation 7. 3-[(phosphonic acid diethyl ester)-methyl]-5-(2-methoxy-(1S)-methyl-ethoxy)-N-(nicotinic acid-3-methyl ester-6-yl)-benzamide

### Step 1: 3-acetoxymethyl-5-(2-methoxy-(1S)-methyl-ethoxy)-N-(nicotinic acid-3-methyl ester-6-yl)-benzamide

3-Acetoxymethyl-5-(2-methoxy-(1S)-methyl-ethoxy)-benzoic acid (1.0 g) prepared in Step 3 of Preparation 4 was dissolved in thionyl chloride (20.0 mL). The reaction mixture was heated at 100° C. for 3 hours and then cooled to room temperature. The reaction mixture was concentrated. The resulting residue was dissolved in pyridine (50.0 mL). 6-Amino-nicotinic acid methyl ester hydrochloride (0.7 g) was added to the solution, which was then stirred at room temperature for 12 hours. The reaction mixture was concentrated and then dichloromethane was added thereto. The reaction mixture was washed with a 1 N hydrochloric acid solution, a saturated sodium hydrogen carbonate solution, distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/2) to obtain 0.9 g of the titled compound in the form of yellow solid (Yield: 62.0%).

¹H-NMR (CDCl₃) δ 9.26 (brs, 1H), 8.82 (d, 1H), 8.46 (d, 1H), 8.34 (dd, 1H), 7.48 (s, 2H), 7.14 (s, 1H), 5.11 (s, 2H), 4.67-4.63 (m, 1H), 3.93 (s, 3H), 3.62-3.41 (m, 2H), 3.40 (s, 3H), 2.12 (s, 3H), 1.33 (d, 3H)

### Step 2: 3-bromomethyl-5-(2-methoxy-(1S)-methyl-ethoxy)-N-(nicotinic acid-3-methyl ester-6-yl)-benzamide

3-Acetoxymethyl-5-(2-methoxy-(1S)-methyl-ethoxy)-N-(nicotinic acid-3-methyl ester-6-yl)-benzamide (0.9 g) prepared in Step 1 was dissolved in a mixed solvent of methanol (40.0 mL) and distilled water (4.0 mL). Potassium carbonate (0.3 g) was added to the solution, which was then stirred at room temperature for 2 hours. The reaction mixture was acidified with a 2 N hydrochloric acid solution and then extracted with ethyl acetate three times. The organic layer was washed with distilled water and brine three times, dried on anhydrous magnesium sulfate, and then concentrated under reduced pressure to obtain 0.5 g of a compound in the form of yellow liquid.

The obtained compound (0.5 g) was dissolved in tetrahydrofuran (20.0 mL). Tribromo phosphate (0.2 mL) was slowly added at 0° C. to the solution, which was then stirred at room temperature for 12 hours. The reaction mixture was concentrated and then ethyl acetate was added thereto. The reaction mixture was washed with a saturated sodium hydrogen carbonate solution, distilled water and brine three times, dried on anhydrous magnesium sulfate, and then concentrated under reduced pressure to obtain 0.2 g of the titled compound in the form of yellow liquid (Yield: 16.4%).

¹H-NMR (CDCl₃) δ 9.82 (brs, 1H), 8.93 (s, 1H), 8.60 (d, 1H), 8.46 (dd, 1H), 7.62 (s, 1H), 7.54 (s, 1H), 7.21 (s, 1H), 4.74-4.69 (m, 1H), 4.50 (s, 2H), 3.97 (s, 3H), 3.60-3.49 (m, 2H), 3.42 (s, 3H), 1.35 (d, 3H)

### Step 3: 3-[(phosphonic acid diethyl ester)-methyl]-5-(2-methoxy-(1S)-methyl-ethoxy)-N-(nicotinic acid-3-methyl ester-6-yl)-benzamide

3-Bromomethyl-5-(2-methoxy-(1S)-methyl-ethoxy)-N-(nicotinic acid-3-methyl ester-6-yl)-benzamide (0.2 g) prepared in Step 2 was dissolved in triethyl phosphite (1.0 mL). The reaction mixture was heated at 160° C. for 3 hours and then cooled to room temperature. The reaction mixture was concentrated under reduced pressure and then purified with silica gel column chromatography (eluent: dichloromethane/methanol=20/1) to obtain 0.2 g of the titled compound in the form of yellow liquid (Yield: 94.3%).

¹H-NMR (CDCl₃) δ 9.10 (brs, 1H), 8.91 (d, 1H), 8.44 (d, 1H), 8.34 (dd, 1H), 7.42 (s, 2H), 7.12 (s, 1H), 4.68-4.62 (m, 1H), 4.11-4.00 (m, 4H), 3.94 (s, 3H), 3.62-3.50 (m, 2H), 3.41 (s, 3H), 3.18 (d, 2H), 1.36-1.25 (m, 9H)

### Preparation 8

### 3-[trans-2-(2-methoxyphenyl)vinyl]-5-(2-methoxy-(1S)-methyl-ethoxy)-benzoic acid

### Step 1: 3-hydroxymethyl-5-(2-methoxy-(1S)-methyl-ethoxy)-benzoic acid methyl ester

5-(2-Methoxy-(1S)-methyl-ethoxy)-isophthalic acid monomethyl ester (30.0 g) prepared in Step 2 of Preparation 3 was dissolved in tetrahydrofuran (600.0 mL). A solution of 1 M tetrahydrofuran-borane complex in tetrahydrofuran (300.0 mL) was slowly added at 0° C. to the solution, which was then stirred at room temperature for 12 hours. Distilled water (900.0 mL) was slowly added at 0° C. to the reaction mixture, which was then stirred for 3 hours. The reaction mixture was concentrated and then extracted with ethyl acetate. The organic layer was washed with distilled water and brine three times, dried on anhydrous magnesium sulfate, and then concentrated under reduced pressure to obtain 26.3 g of the titled compound in the form of yellow liquid (Yield: 92.5%).

¹H-NMR (CDCl₃) δ 7.61 (s, 1H), 7.50 (t, 1H), 7.16 (s, 1H), 4.69 (s, 2H), 4.65-4.60 (m, 1H), 3.90 (s, 3H), 3.61-3.48 (m, 2H), 3.41 (s, 3H), 1.93 (brs, 1H), 1.31 (d, 3H)

### Step 2: 3-bromomethyl-5-(2-methoxy-(1S)-methyl-ethoxy)-benzoic acid methyl ester

3-Hydroxymethyl-5-(2-methoxy-(1S)-methyl-ethoxy)-benzoic acid methyl ester (6.3 g) prepared in Step 1 was dissolved in tetrahydrofuran (300.0 mL). Tribromo phosphate (2.6 mL) was slowly added at 0° C. to the solution, which was then stirred at room temperature for 12 hours. The reaction mixture was concentrated and then ethyl acetate was added thereto. The reaction mixture was washed with a saturated sodium hydrogen carbonate solution and brine three times, dried on anhydrous magnesium sulfate, and then concentrated under reduced pressure to obtain a residue in the form of yellow liquid. The residue was purified with silica gel column chromatography (eluent: n-hexane/ethyl acetate=2/1) to obtain 5.2 g of the titled compound in the form of yellow liquid (Yield: 66.1%).

¹H-NMR (CDCl₃) δ 7.64 (t, 1H), 7.52 (dd, 1H), 7.16 (t, 1H), 4.64-4.59 (m, 1H), 4.42 (s, 2H), 3.91 (s, 3H), 3.61-3.48 (m, 2H), 3.42 (s, 3H), 1.32 (d, 3H)

### Step 3: 3-[(phosphonic acid diethyl ester)-methyl]-5-(2-methoxy-(1S)-methyl-ethoxy)-benzoic acid methyl ester

3-Bromomethyl-5-(2-methoxy-(1S)-methyl-ethoxy)-benzoic acid methyl ester (5.2 g) prepared in Step 2 was dissolved in triethyl phosphite (14.3 mL). The reaction mixture was heated at 160° C. for 3 hours and then cooled to room temperature. The reaction mixture was concentrated under reduced pressure and then purified with silica gel column chromatography (eluent: dichloromethane/methanol=20/1) to obtain 6.1 g of the titled compound in the form of yellow liquid (Yield: 99.2%).

¹H-NMR (CDCl₃) δ 7.54 (d, 1H), 7.49 (d, 1H), 7.10 (d, 1H), 4.62-4.58 (m, 1H), 4.13-4.00 (m, 4H), 3.89 (s, 3H), 3.60-3.47 (m, 2H), 3.41 (s, 3H), 3.14 (d, 2H), 1.31 (d, 3H), 1.28-1.24 (m, 6H)

### Step 4

### 3-[trans-2-(2-methoxyphenyl)vinyl]-5-(2-methoxy-(1S)-methyl-ethoxy)-benzoic acid

3-[(Phosphonic acid diethyl ester)-methyl]-5-(2-methoxy-(1S)-methyl-ethoxy)-benzoic acid methyl ester (1.0 g) prepared in Step 3, potassium hydroxide (0.5 g), and 18-crown-6 (0.4 g) were dissolved in dichloromethane (20.0 mL). 2-Methoxybenzaldehyde (0.4 mL) was added to the solution, which was then stirred at room temperature for 20 hours. The reaction mixture was concentrated, acidified with a 1 N hydrochloric acid solution, and then extracted with ethyl acetate three times. The organic layer was washed with distilled water and brine three times, dried on anhydrous magnesium sulfate and then concentrated under reduced pressure to obtain a residue in the form of yellow liquid. The residue was purified with silica gel column chromatography (eluent: dichloromethane/methanol=20/1) to obtain 0.9 g of the titled compound in the form of white solid (Yield: 99.9%).

¹H-NMR (CDCl₃) δ 7.87 (s, 1H), 7.59-7.51 (m, 3H), 7.35 (s, 1H), 7.28-7.24 (m, 1H), 7.09 (d, 1H), 6.99-6.90 (m, 2H), 4.70-4.65 (m, 1H), 3.90 (s, 3H), 3.65-3.52 (m, 2H), 3.44 (s, 3H), 1.36 (d, 3H)

### Preparation 9

### 3-[2-(2,6-difluorophenyl)ethyl]-5-(2-methoxy-(1S)-methyl-ethoxy)-N-(nicotinic acid-3-methyl ester-6-yl)-benzamide

### Step 1

### 3-[trans-2-(2,6-difluorophenyl)vinyl]-5-(2-methoxy-(1S)-methyl-ethoxy)-benzoic acid

3-[(Phosphonic acid diethyl ester)-methyl]-5-(2-methoxy-(1S)-methyl-ethoxy)-benzoic acid methyl ester (6.0 g) prepared in Step 2 of Preparation 5, potassium hydroxide (2.8 g), and 18-crown-6 (2.2 g) was dissolved in dichloromethane (30.0 mL). 2,6-Difluorobenzaldehyde (2.8 g) was added to the solution, which was then stirred at room temperature for 16 hours. The reaction mixture was washed with distilled water and brine three times, dried on anhydrous magnesium sulfate, and then concentrated under reduced pressure to obtain a residue in the form of yellow liquid. The residue was purified with silica gel column chromatography (eluent: dichloromethane/methanol=20/1) to obtain 0.9 g of the titled compound in the form of yellow liquid (Yield: 15.0%).

¹H-NMR (CDCl₃) δ 7.86 (s, 1H), 7.58 (s, 1H), 7.43 (d, 1H), 7.36 (s, 1H), 7.22-7.17 (m, 1H), 7.19 (d, 1H), 6.93 (t, 2H), 4.70-4.66 (m, 1H), 3.65-3.53 (m, 2H), 3.44 (s, 3H), 1.36 (d, 3H)

### Step 2: 3-[2-(2,6-difluorophenyl)ethyl]-5-(2-methoxy-(1S)-methyl-ethoxy)-benzoic acid

3-[trans-2-(2,6-Difluorophenyl)vinyl]-5-(2-methoxy-(1S)-methyl-ethoxy)-benzoic acid (888.0 mg) prepared in Step 1 was dissolved in methanol (5.0 mL) and then Pd/C (200.0 mg) was added thereto. Hydrogenation reaction was performed by stirring the solution at room temperature for 24 hours, using a hydrogen balloon. The reaction mixture was filtered with celite pad. The filtrate was concentrated under reduced pressure to obtain 780.0 mg of the titled compound in the form of yellow liquid (Yield: 87.0%).

¹H-NMR (CDCl₃) δ 9.23 (brs, 1H), 7.56 (s, 1H), 7.51 (s, 1H), 7.20-7.09 (m, 1H), 7.03 (s, 1H), 6.85 (t, 2H), 4.69-4.52 (m, 1H), 3.69-3.50 (m, 2H), 3.44 (s, 3H), 2.97-2.01 (m, 4H), 1.31 (d, 3H)

### Step 3: 3-[2-(2,6-difluorophenyl)ethyl]-5-(2-methoxy-(1S)-methyl-ethoxy)-N-(nicotinic acid-3-methyl ester-6-yl)-benzamide

3-[2-(2,6-Difluorophenyl)ethyl]-5-(2-methoxy-(1S)-methyl-ethoxy)-benzoic acid (780.0 mg) prepared in Step 2 was dissolved in dichloromethane (5.0 mL). Oxalyl chloride (310.8 mg) and one drop of N,N-dimethylformamide were added to the solution, which was then stirred at room temperature for 24 hours. The reaction mixture was concentrated and then the resulting reside was dissolved in pyridine (5.0 mL). 6-Aminonicotinic acid methyl ester hydrochloride (558.5 mg) was added to the solution, which was then stirred at room temperature for 24 hours. The reaction mixture was concentrated and then dichloromethane was added thereto. The reaction mixture was washed with a 1 N hydrochloric acid solution, a saturated sodium hydrogen carbonate solution, distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=2/1) to obtain 740.0 mg of the titled compound in the form of white solid (Yield: 70.0%).

¹H-NMR (CDCl₃) δ 7.52 (s, 1H), 7.50 (s, 1H), 7.15 (t, 1H), 7.08 (s, 1H), 6.84 (t, 2H), 4.61-4.56 (m, 1H), 3.60-3.48 (m, 2H), 3.42 (s, 3H), 2.99-2.89 (m, 4H), 1.30 (d, 3H)

### Preparation 10

3-[trans-2-(4-fluorophenyl)vinyl]-5-(1-methoxymethyl-propoxy)-benzoic acid

### Step 1: 5-(2-methyl-benzyloxy)-isophthalic acid dimethyl ester

Dimethyl 5-hydroxyisophthalate (28.0 g) and 2-methylbenzyl bromide (25.0 g), and potassium carbonate (28.0 g) were dissolved in N,N-dimethylformamide (150.0 mL). The reaction mixture was heated at 50° C. for 12 hours and then cooled to room temperature. The reaction mixture was concentrated and then ethyl acetate was added thereto. The reaction mixture was washed with distilled water and brine three times, dried on anhydrous magnesium sulfate, and then concentrated under reduced pressure to obtain 40.8 g of the titled compound in the form of white solid (Yield: 96.1%).

¹H-NMR (CDCl₃) δ 8.31 (t, 1H), 7.85 (d, 2H), 7.42 (d, 1H), 7.28-7.23 (m, 3H), 5.12 (s, 2H), 3.95 (s, 6H), 2.39 (s, 3H)

### Step 2: 5-(2-methyl-benzyloxy)-isophthalic acid monomethyl ester

5-(2-Methyl-benzyloxy)-isophthalic acid dimethyl ester (40.0 g) prepared in Step 1 was dissolved in methanol (300.0 mL). Potassium hydroxide (6.4 g) was added to the solution, which was then stirred at 100° C. for 12 hours. The reaction mixture was concentrated and then distilled water was added thereto. The reaction mixture was washed with ethyl acetate three times, acidified with a 2 N hydrochloric acid solution, and then extracted with ethyl acetate three times. The organic layer was washed with distilled water and brine three times, dried on anhydrous magnesium sulfate, and then concentrated under reduced pressure to obtain 28.0 g of the titled compound in the form of white solid (Yield: 73.3%).

¹H-NMR (d₆-DMSO) δ 8.13 (d, 1H), 7.81-7.78 (m, 2H), 7.46 (d, 1H), 7.28-7.24 (m, 3H), 5.24 (s, 2H), 3.91 (s, 3H), 2.37 (s, 3H)

### Step 3: 3-hydroxymethyl-5-(2-methyl-benzyloxy)-benzoic acid methyl ester

5-(2-Methyl-benzyloxy)-isophthalic acid monomethyl ester (8.0 g) prepared in Step 2 was dissolved in tetrahydrofuran (250.0 mL). A solution of 1 M tetrahydrofuran-borane complex in tetrahydrofuran (100.0 mL) was slowly added at 0° C. to the solution, which was then stirred at room temperature for 12 hours. Distilled water (500.0 mL) was slowly added at 0° C. to the reaction mixture, which was then stirred for 3 hours. The reaction mixture was concentrated and then extracted with ethyl acetate. The organic layer was washed with distilled water and brine three times, dried on anhydrous magnesium sulfate, and then concentrated under reduced pressure to obtain 5.0 g of the titled compound in the form of yellow liquid (Yield: 65.5%).

¹H-NMR (CDCl₃) δ 7.64 (s, 1H), 7.59 (d, 1H), 7.41 (dd, 1H), 7.28-7.21 (m, 4H), 5.08 (s, 2H), 4.71 (s, 2H), 3.91 (s, 3H), 2.38 (s, 3H)

### Step 4: 3-bromomethyl-5-(2-methyl-benzyloxy)-benzoic acid methyl ester

3-Hydroxymethyl-5-(2-methyl-benzyloxy)-benzoic acid methyl ester (5.0 g) prepared in Step 3 was dissolved in tetrahydrofuran (100.0 mL). Tribromo phosphate (2.0 mL) was slowly added at 0° C. to the solution, which was then stirred at room temperature for 12 hours. The reaction mixture was concentrated and then ethyl acetate was added thereto. The reaction mixture was washed with a saturated sodium hydrogen carbonate solution, distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=5/1) to obtain 3.7 g of the titled compound in the form of pale yellow liquid (Yield: 60.7%).

¹H-NMR (CDCl₃) δ 7.68 (d, 1H), 7.60 (t, 1H), 7.40 (d, 1H), 7.28-7.21 (m, 4H), 5.08 (s, 2H), 4.48 (s, 2H), 3.92 (s, 3H), 2.39 (s, 3H)

### Step 5: 3-[(phosphonic acid diethyl ester)-methyl]-5-(2-methyl-benzyloxy)-benzoic acid methyl ester

3-bromomethyl-5-(2-methyl-benzyloxy)-benzoic acid methyl ester (3.7 g) prepared in Step 4 was dissolved in triethyl phosphite (9.3 mL). The reaction mixture was heated at 160° C. for 3 hours and then cooled to room temperature. The reaction mixture was concentrated under reduced pressure, and then purified with silica gel column chromatography (eluent: dichloromethane/methanol=20/1) to obtain 3.6 g of the titled compound in the form of yellow liquid (Yield: 79.2%).

¹H-NMR (CDCl₃) δ 7.58 (d, 2H), 7.41 (d, 1H), 7.29-7.22 (m, 3H), 7.16 (d, 1H), 5.07 (s, 2H), 4.11-4.00 (m, 4H), 3.91 (s, 3H), 3.16 (d, 2H), 2.38 (s, 3H), 1.26 (t, 6H)

### Step 6: 3-[trans-2-(4-fluorophenyl)vinyl]-5-(2-methyl-benzyloxy)-benzoic acid

3-[(Phosphonic acid diethyl ester)-methyl]-5-(2-methyl-benzyloxy)-benzoic acid methyl ester (3.6 g) prepared in Step 5, potassium hydroxide (1.0 g), and 18-crown-6 (0.2 g) were dissolved in dichloromethane (100.0 mL). 4-Fluorobenzaldehyde (1.0 mL) was added to the solution, which was then stirred at room temperature for 17 hours. The reaction mixture was washed with distilled water and brine three times, dried on anhydrous magnesium sulfate, and then concentrated under reduced pressure to obtain a residue in the form of yellow liquid residue. The residue was purified with silica gel column chromatography (eluent: dichloromethane/methanol=20/1) to obtain 3.4 g of the titled compound in the form of yellow liquid (Yield: 99.9%).

¹H-NMR (CDCl₃) δ 7.90 (s, 1H), 7.63 (t, 1H), 7.51-7.47 (m, 3H), 7.35 (d, 1H), 7.30-7.22 (m, 3H), 7.13-7.03 (m, 4H), 5.13 (s, 2H), 2.41 (s, 3H)

### Step 7: 3-[trans-2-(4-fluorophenyl)vinyl]-5-(2-methyl-benzyloxy)-benzoic acid methyl ester

3-[trans-2-(4-Fluorophenyl)vinyl]-5-(2-methyl-benzyloxy)-benzoic acid (3.4 g) prepared in Step 6 was dissolved in N,N-dimethylformamide (34.0 mL). Potassium carbonate (3.9 g) and methyl iodide (1.2 mL) were added to the solution, which was then stirred at room temperature for 12 hours. Ethyl acetate was added to the reaction mixture, which was washed with distilled water and brine three times, dried on anhydrous magnesium sulfate, and then concentrated under reduced pressure to obtain 3.9 g of the titled compound in the form of yellow liquid (Yield: 99.9%).

¹H-NMR (CDCl₃) δ 7.82 (s, 1H), 7.56 (d, 1H), 7.50-7.44 (m, 3H), 7.29-7.24 (m, 4H), 7.10-7.00 (m, 4H), 5.11 (s, 2H), 3.94 (s, 3H), 2.40 (s, 3H)

### Step 8: 3-[trans-2-(4-fluorophenyl)vinyl]-5-hydroxy-benzoic acid methyl ester

3-[trans-2-(4-Fluorophenyl)vinyl]-5-(2-methyl-benzyloxy)-benzoic acid methyl ester (3.9 g) prepared in Step 7 and thioanisole (6.7 mL) were dissolved in trifluoroacetic acid (43.7 mL). The reaction mixture was stirred at room temperature for 12 hours. The reaction mixture was concentrated and then purified with silica gel column chromatography (eluent: n-hexane/ethyl acetate=1/2) to obtain 2.1 g of the titled compound in the form of yellow liquid (Yield: 71.5%).

¹H-NMR (CDCl₃) δ 7.34 (s, 1H), 7.30 (t, 1H), 7.15-7.07 (m, 4H), 6.92 (t, 2H), 6.63 (d, 1H), 5.34 (s, 1H), 3.87 (s, 3H)

### Step 9: 3-[trans-2-(4-fluorophenyl)vinyl]-5-(1-methoxymethyl-propoxy)-benzoic acid methyl ester

3-[trans-2-(4-Fluorophenyl)vinyl]-5-hydroxy-benzoic acid methyl ester (100.0 mg) prepared in Step 8,1-methoxy-2-butanol (43.0 μL), and triphenylphosphine (120.0 mg) were dissolved in tetrahydrofuran (2.0 mL). DIAD (90.0 μL) was slowly added at 0° C. to the solution, which was then stirred at room temperature for 12 hours. The reaction mixture was concentrated and then purified with silica gel column chromatography (eluent: n-hexane/ethyl acetate=5/1) to obtain 113.0 mg of the titled compound in the form of yellow liquid (Yield: 85.1%).

¹H-NMR (CDCl₃) δ 7.38 (d, 2H), 7.20-7.07 (m, 4H), 6.92 (t, 2H), 6.67 (s, 1H), 4.20-4.16 (m, 1H), 3.87 (s, 3H), 3.48-3.44 (m, 2H), 3.35 (s, 3H), 1.65-1.59 (m, 2H), 0.90 (t, 3H)

### Step 10: 3-[trans-2-(4-fluorophenyl)vinyl]-5-(1-methoxymethyl-propoxy)-benzoic acid

### 3-[trans-2-(4-Fluorophenyl)vinyl]-5-(1-methoxymethyl-propoxy)-benzoic acid methyl ester (113.0 mg) prepared in Step 9 was dissolved in a mixed solvent of tetrahydrofuran (2.0 mL) and methanol (1.0 mL). A 3 N sodium hydroxide solution (2.0 mL) was added to the solution, which was then stirred at room temperature for 12 hours. The reaction mixture was concentrated, acidified with a 1 N hydrochloric acid solution, and then extracted with ethyl acetate. The organic layer was dried on anhydrous magnesium sulfate and then concentrated under reduced pressure to obtain 99.0 mg of the titled compound in the form of yellow liquid (Yield: 91.3%).

¹H-NMR (CDCl₃) δ 7.46 (s, 2H), 7.17-7.07 (m, 4H), 6.94-6.90 (m, 2H), 6.72 (d, 1H), 4.20-4.17 (m, 1H), 3.52-3.46 (m, 2H), 2.36 (s, 3H), 1.66-1.60 (m, 2H), 0.91 (t, 3H)

### Example 1

### 3-[trans-2-(p-tolyl)vinyl]-5-isobutoxy-N-(thiazol-2-yl)-benzamide

3-Bromo-5-isobutoxy-N-(thiazol-2-yl)-benzamide (100.0 mg) prepared in Preparation 1 and trans-2-(4-methylphenyl)vinylboric acid (92.0 mg) were dissolved in a mixed solvent (5.5 mL) of toluene and ethanol (1/0.1). A 2 M potassium carbonate solution (0.6 mL) and tetrakis(triphenylphosphine)palladium(0) (10.0 mg) were added to the solution, which was then stirred at 100° C. for 12 hours. The reaction mixture was filtered with celite pad. The filtrate was concentrated under reduced pressure. The resulting residue was dissolved in ethyl acetate. The solution was washed with distilled water, dried on anhydrous magnesium sulfate, and then concentrated under reduced pressure to obtain a residue in the form of yellow liquid residue. The residue was purified with silica gel column chromatography (eluent: n-hexane/ethyl acetate=5/1) to obtain 70.0 mg of the titled compound in the form of yellow liquid (Yield: 83.1%).

¹H-NMR (CDCl₃) δ 12.47 (brs, 1H), 7.69 (d, 1H), 7.47-6.94 (m, 10H), 3.79 (d, 2H), 2.37 (s, 3H), 2.14-2.06 (m, 1H), 1.05 (d, 6H)

### Examples 2 and 3

The titled compounds of Examples 2 and 3 were prepared, in accordance with the same procedures as in Example 1, using 3-bromo-5-isobutoxy-N-(thiazol-2-yl)-benzamide prepared in Preparation 1; and trans-2-(4-fluorophenyl)vinylboric acid or trans-2-(2-nitrophenyl)vinylboric acid, instead of trans-2-(4-methylphenyl)vinylboric acid, respectively.

### Example 2

### 3-[trans-2-(4-fluorophenyl)vinyl]-5-isobutoxy-N-(thiazol-2-yl)-benzamide

Yield: 44.7%

¹H-NMR (CDCl₃) δ 12.96 (brs, 1H), 7.70 (d, 1H), 7.45-7.24 (m, 4H), 7.12-6.92 (m, 6H), 3.78 (d, 2H), 2.13-2.07 (m, 1H), 1.04 (d, 6H)

### Example 3

### 3-[trans-2-(2-nitrophenyl)vinyl]-5-isobutoxy-N-(thiazol-2-yl)-benzamide

Yield: 40.9%

¹H-NMR (CDCl₃) δ 8.03 (d, 1H), 7.70 (m, 2H), 7.61 (m, 2H), 7.45-7.50 (m, 2H), 7.30 (s, 1H), 7.12 (d, 1H), 7.01 (d, 1H), 6.93 (d, 1H), 4.10 (d, 2H), 2.13 (m, 1H), 1.04 (d, 6H)

### Example 4

### 3-[trans-2-(3,4-dimethoxyphenyl)vinyl]-5-isobutoxy-N-(thiazol-2-yl)-benzamide

### Step 1: 3-[(phosphonic acid diethyl ester)-methyl]-5-isobutoxy-N-(thiazol-2-yl)-benzamide

3-Bromomethyl-5-isobutoxy-N-(thiazol-2-yl)-benzamide (0.8 g) prepared in Step 5 of Preparation 2 was dissolved in triethyl phosphite (2.0 mL). The reaction mixture was heated at 160° C. for 3 hours and then cooled to room temperature. The reaction mixture was concentrated under reduced pressure and then purified with silica gel column chromatography (eluent: dichloromethane/methanol=20/1) to obtain 0.6 g of the titled compound in the form of white solid (Yield: 66.7%).

¹H-NMR (CDCl₃) δ 11.71 (brs, 1H), 7.42 (d, 2H), 7.23 (d, 1H), 7.13 (d, 1H), 6.97 (d, 1H), 4.09-4.00 (m, 4H), 3.74 (d, 2H), 3.15 (d, 2H), 2.10-2.04 (m, 1H), 1.27 (t, 6H), 1.01 (d, 6H)

### Step 2

### 3-[trans-2-(3,4-dimethoxyphenyl)vinyl]-5-isobutoxy-N-(thiazol-2-yl)-benzamide

3-[(Phosphonic acid diethyl ester)-methyl]-5-isobutoxy-N-(thiazol-2-yl)-benzamide (100.0 mg) prepared in Step 1, potassium hydroxide (27.0 mg), and 18-crown-6 (6.0 mg) were dissolved in dichloromethane (2.0 mL). 3,4-Dimethoxybenzaldehyde (43.0 mg) was added to the solution, which was then stirred at room temperature for 6 hours. The reaction mixture was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=2/1) to obtain 70.0 mg of the titled compound in the form of yellow liquid (Yield: 68.2%).

¹H-NMR (CDCl₃) δ 12.84 (brs, 1H), 7.72 (s, 1H), 7.44 (d, 1H), 7.26 (d, 1H), 7.16 (d, 1H), 7.01-6.85 (m, 6H), 3.95 (s, 3H), 3.91 (s, 3H), 3.79 (d, 2H), 2.17-2.07 (m, 1H), 1.05 (d, 6H)

### Example 5

### 3-[trans-2-(2,3-dimethoxyphenyl)vinyl]-5-isobutoxy-N-(thiazol-2-yl)-benzamide

The titled compound was prepared, in accordance with the same procedures as in Step 2 of Example 4, using 3-[(phosphonic acid diethyl ester)-methyl]-5-isobutoxy-N-(thiazol-2-yl)-benzamide prepared in Step 1 of Example 4; and 2,3-dimethoxybenzaldehyde instead of 3,4-dimethoxybenzaldehyde.

Yield: 33.1%

¹H-NMR (CDCl₃) δ 12.23 (brs, 1H), 7.74 (s, 1H), 7.50-7.42 (m, 2H), 7.32 (d, 1H), 7.19-7.17 (m, 2H), 7.11-7.06 (m, 2H), 6.94 (d, 1H), 6.87 (d, 1H), 3.89 (s, 3H), 3.84 (s, 3H), 3.80 (d, 2H), 2.16-2.08 (m, 1H), 1.05 (d, 6H)

### Example 6

### 3-[trans-2-(2-fluorophenyl)vinyl]-5-(2-methoxy-(1S)-methyl-ethoxy)-N-(thiazol-2-yl)-benzamide

3-[(Phosphonic acid diethyl ester)-methyl]-5-(2-methoxy-(1S)-methyl-ethoxy)-N-(thiazol-2-yl)-benzamide (30.0 mg) prepared in Preparation 3, potassium hydroxide (8.0 mg), and 18-crown-6 (9.0 mg) were dissolved in dichloromethane (1.0 mL). 2-Fluorobenzaldehyde (8.0 mL) was added to the solution, which was then stirred at room temperature for 20 hours. The reaction mixture was washed with distilled water and brine three times, dried on anhydrous magnesium sulfate, and then concentrated under reduced pressure to obtain a residue in the form of yellow liquid residue. The residue was purified with silica gel column chromatography (eluent: n-hexane/ethyl acetate=1/1) to obtain 12.0 mg of the titled compound in the form of yellow liquid (Yield: 44.5%).

¹H-NMR (CDCl₃) δ 12.21 (brs, 1H), 7.72 (s, 1H), 7.50 (s, 1H), 7.35-7.10 (m, 8H), 6.94 (d, 1H), 4.70-4.60 (m, 1H), 3.64-3.51 (m, 2H), 3.43 (s, 3H), 1.36 (d, 3H)

### Examples 7 to 18

The titled compounds of Examples 7 to 18 were prepared, in accordance with the same procedures as in Example 6, using 3-[(phosphonic acid diethyl ester)-methyl]-5-(2-methoxy-(1S)-methyl-ethoxy)-N-(thiazol-2-yl)-benzamide prepared in Preparation 3; and aldehydes suitable for Examples 7 to 18, instead of 2-fluorobenzaldehyde, respectively.

### Example 7

### 3-[trans-2-(3-fluorophenyl)vinyl]-5-(2-methoxy-(1S)-methyl-ethoxy)-N-(thiazol-2-yl)-benzamide

Yield: 44.5%

¹H-NMR (CDCl₃) δ 12.07 (brs, 1H), 7.71 (s, 1H), 7.51 (s, 1H), 7.32-6.95 (m, 9H), 4.70-4.60 (m, 1H), 3.64-3.51 (m, 2H), 3.43 (s, 3H), 1.36 (d, 3H)

### Example 8

### 3-[trans-2-(4-fluorophenyl)vinyl]-5-(2-methoxy-(1S)-methyl-ethoxy)-N-(thiazol-2-yl)-benzamide

Yield: 46.7%

¹H-NMR (CDCl₃) δ 7.80 (s, 1H), 7.51 (s, 1H), 7.42 (dd, 2H), 7.34 (s, 1H), 7.21 (d, 1H), 7.10 (dd, 2H), 7.00 (s, 4H), 6.93 (d, 1H), 4.65 (m, 1H), 3.64 (m, 2H), 3.43 (s, 3H), 1.34 (d, 3H)

### Example 9

### 3-[trans-2-(2,3-difluorophenyl)vinyl]-5-(2-methoxy-(1S)-methyl-ethoxy)-N-(thiazol-2-yl)-benzamide

Yield: 42.6%

¹H-NMR (CDCl₃) δ 12.10 (brs, 1H), 7.71 (s, 1H), 7.51 (s, 1H), 7.32-6.95 (m, 8H), 4.70-4.60 (m, 1H), 3.64-3.51 (m, 2H), 3.43 (s, 3H), 1.35 (d, 3H)

### Example 10

### 3-[trans-2-(2,4-difluorophenyl)vinyl]-5-(2-methoxy-(1S)-methyl-ethoxy)-N-(thiazol-2-yl)-benzamide

Yield: 42.6%

¹H-NMR (CDCl₃) δ 7.70 (s, 1H), 7.50 (s, 1H), 7.32 (s, 1H), 7.18-7.13 (m, 2H), 7.03 (d, 1H), 6.95-6.82 (m, 4H), 4.70-4.60 (m, 1H), 3.64-3.51 (m, 2H), 3.42 (s, 3H), 1.35 (d, 3H)

### Example 11

### 3-[trans-2-(2,5-difluorophenyl)vinyl]-5-(2-methoxy-(1S)-methyl-ethoxy)-N-(thiazol-2-yl)-benzamide

Yield: 35.5%

¹H-NMR (CDCl₃) δ 7.74 (s, 1H), 7.51 (m, 1H), 7.32 (d, 2H), 7.23 (s, 1H), 7.13-6.90 (m, 5H), 4.70-4.60 (m, 1H), 3.64-3.52 (m, 2H), 3.43 (s, 3H), 1.36 (d, 3H)

### Example 12

### 3-[trans-2-(2,6-difluorophenyl)vinyl]-5-(2-methoxy-(1S)-methyl-ethoxy)-N-(thiazol-2-yl)-benzamide

Yield: 35.5%

¹H-NMR (CDCl₃) δ 7.75 (s, 1H), 7.52 (d, 1H), 7.40 (d, 1H), 7.34 (d, 1H), 7.29 (d, 1H), 7.20-7.15 (m, 2H), 6.98 (d, 1H), 6.93 (t, 2H), 4.70-4.60 (m, 1H), 3.64-3.52 (m, 2H), 3.43 (s, 3H), 1.36 (d, 3H)

### Example 13

### 3-[trans-2-(2-nitrophenyl)vinyl]-5-(2-methoxy-(1S)-methyl-ethoxy)-N-(thiazol-2-yl)-benzamide

Yield: 34.7%

¹H-NMR (CDCl₃) δ 11.49 (brs, 1H), 7.99 (d, 1H), 7.71-7.60 (m, 4H), 7.52 (m, 1H), 7.45 (t, 1H), 7.38 (d, 1H), 7.25 (s, 1H), 7.03-6.97 (m, 2H), 4.70-4.60 (m, 1H), 3.64-3.52 (m, 2H), 3.43 (s, 3H), 1.36 (d, 3H)

### Example 14

### 3-[trans-2-(3-nitrophenyl)vinyl]-5-(2-methoxy-(1S)-methyl-ethoxy)-N-(thiazol-2-yl)-benzamide

Yield: 34.7%

¹H-NMR (CDCl₃) δ 8.37 (s, 1H), 8.14 (d, 1H), 7.78 (d, 1H), 7.73 (s, 1H), 7.58-7.50 (m, 3H), 7.35-7.32 (m, 2H), 7.18 (d, 1H), 7.01 (d, 1H), 4.70-4.60 (m, 1H), 3.64-3.52 (m, 2H), 3.43 (s, 3H), 1.36 (d, 3H)

### Example 15

### 3-[trans-2-(4-nitrophenyl)vinyl]-5-(2-methoxy-(1S)-methyl-ethoxy)-N-(thiazol-2-yl)-benzamide

Yield: 34.7%

¹H-NMR (CDCl₃) δ 11.41 (brs, 1H), 8.26-8.23 (m, 2H), 7.74 (s, 1H), 7.61 (d, 2H), 7.52 (s, 1H), 7.33 (d, 1H), 7.29 (d, 1H), 7.17 (q, 2H), 6.99 (d, 1H), 4.70-4.60 (m, 1H), 3.64-3.52 (m, 2H), 3.43 (s, 3H), 1.36 (d, 3H)

### Example 16

### 3-[trans-2-(2-methoxyphenyl)vinyl]-5-(2-methoxy-(1S)-methyl-ethoxy)-N-(thiazol-2-yl)-benzamide

Yield: 36.0%

¹H-NMR (CDCl₃) δ 11.50 (brs, 1H), 7.72 (s, 1H), 7.51-7.44 (m, 4H), 7.33 (d, 1H), 7.07 (d, 1H), 6.98-6.90 (m, 4H), 4.70-4.60 (m, 1H), 3.88 (s, 3H), 3.64-3.52 (m, 2H), 3.43 (s, 3H), 1.36 (d, 3H)

### Example 17

### 3-[trans-2-(2,3-dimethoxyphenyl)vinyl]-5-(2-methoxy-(1S)-methyl-ethoxy)-N-(thiazol-2-yl)-benzamide

Yield: 33.5%

¹H-NMR (CDCl₃) δ 11.67 (brs, 1H), 7.71 (s, 1H), 7.50-7.46 (m, 2H), 7.37 (s, 1H), 7.24-7.18 (m, 2H), 7.10-7.05 (m, 2H), 6.96 (d, 1H), 6.87 (d, 1H), 4.70-4.60 (m, 1H), 3.90 (s, 3H), 3.84 (s, 3H), 3.64-3.52 (m, 2H), 3.43 (s, 3H), 1.36 (d, 3H)

### Example 18

### 3-[trans-2-(2,3,5-trifluorophenyl)vinyl]-5-(2-methoxy-(1S)-methyl-ethoxy)-N-(thiazol-2-yl)-benzamide

Yield: 23.8%

¹H-NMR (CDCl₃) δ 7.66 (d, 1H), 7.46 (s, 1H), 7.32 (d, 1H), 7.16-6.94 (m, 6H), 4.70-4.60 (m, 1H), 3.64-3.51 (m, 2H), 3.43 (s, 3H), 1.36 (d, 3H)

### Example 19

### 3-[trans-2-(2-nitrophenyl)vinyl]-5-(2-methoxy-(1S)-methyl-ethoxy)-N-(5-fluorothiazol-2-yl)-benzamide

3-[(Phosphonic acid diethyl ester)-methyl]-5-(2-methoxy-(1S)-methyl-ethoxy)-N-(5-fluorothiazol-2-yl)-benzamide (30.0 mg) prepared in Preparation 4, potassium hydroxide (8.0 mg), and 18-crown-6 (9.0 mg) were dissolved in dichloromethane (1.0 mL). 2-Nitrobenzaldehyde (11.0 mg) was added to the solution, which was then stirred at room temperature for 20 hours. The reaction mixture was washed with distilled water and brine, dried on anhydrous magnesium sulfate, and then concentrated under reduced pressure to obtain a residue in the form of yellow liquid. The residue was purified with silica gel column chromatography (eluent: n-hexane/ethyl acetate=2/1) to obtain 1.0 mg of the titled compound in the form of yellow liquid (Yield: 3.4%).

¹H-NMR (CDCl₃) δ 10.10 (brs, 1H), 8.01 (d, 1H), 7.74 (d, 1H), 7.67-7.60 (m, 3H), 7.48-7.42 (m, 2H), 7.37 (s, 1H), 7.05-6.97 (m, 2H), 4.71-4.65 (m, 1H), 3.5-3.52 (m, 2H), 3.43 (s, 3H), 1.37 (d, 3H)

### Examples 20 to 31

The titled compounds of Examples 20 to 31 were prepared, in accordance with the same procedures as in Example 19, using 3-[(phosphonic acid diethyl ester)-methyl]-5-(2-methoxy-(1S)-methyl-ethoxy)-N-(5-fluorothiazol-2-yl)-benzamide prepared in Preparation 4; and aldehydes suitable for Examples 20 to 31, instead of 2-nitrobenzaldehyde, respectively.

### Example 20

### 3-[trans-2-(3-nitrophenyl)vinyl]-5-(2-methoxy-(1S)-methyl-ethoxy)-N-(5-fluorothiazol-2-yl)-benzamide

Yield: 10.2%

¹H-NMR (CDCl₃) δ 10.94 (brs, 1H), 8.37 (t, 1H), 8.16-8.13 (m, 1H), 7.78 (d, 1H), 7.66 (s, 1H), 7.58-7.52 (m, 1H), 7.44 (s, 1H), 7.34 (s, 1H), 7.18 (d, 2H), 6.91 (d, 1H), 4.71-4.66 (m, 1H), 3.65-3.53 (m, 2H), 3.44 (s, 3H), 1.37 (d, 3H)

### Example 21

### 3-[trans-2-(4-nitrophenyl)vinyl]-5-(2-methoxy-(1S)-methyl-ethoxy)-N-(5-fluorothiazol-2-yl)-benzamide

Yield: 6.8%

¹H-NMR (CDCl₃) δ 10.45 (brs, 1H), 8.25 (d, 2H), 7.66-7.62 (m, 3H), 7.44 (d, 1H), 7.34 (d, 1H), 7.20 (d, 2H), 6.94 (d, 1H), 4.71-4.65 (m, 1H), 3.65-3.53 (m, 2H), 3.44 (s, 3H), 1.37 (d, 3H)

### Example 22

### 3-[trans-2-(2-fluorophenyl)vinyl]-5-(2-methoxy-(1S)-methyl-ethoxy)-N-(5-fluorothiazol-2-yl)-benzamide

Yield: 35.7%

¹H-NMR (CDCl₃) δ 11.75 (brs, 1H), 7.65 (s, 1H), 7.58-7.54 (m, 1H), 7.42 (t, 1H), 7.34 (s, 1H), 7.29-7.21 (m, 2H), 7.18-7.05 (m, 3H), 6.77 (d, 1H), 4.70-4.65 (m, 1H), 3.65-3.52 (m, 2H), 3.43 (s, 3H), 1.36 (d, 3H)

### Example 23

### 3-[trans-2-(4-fluorophenyl)vinyl]-5-(2-methoxy-(1S)-methyl-ethoxy)-N-(5-fluorothiazol-2-yl)-benzamide

Yield: 28.6%

¹H-NMR (CDCl₃) δ 11.60 (brs, 1H), 7.62 (s, 1H), 7.48-7.39 (m, 3H), 7.29 (t, 1H), 7.09-7.14 (m, 3H), 6.95 (d, 1H), 6.78 (d, 1H), 4.70-4.63 (m, 1H), 3.64-3.51 (m, 2H), 3.43 (s, 3H), 1.36 (d, 3H)

### Example 24

### 3-[trans-2-(2,3-difluorophenyl)vinyl]-5-(2-methoxy-(1S)-methyl-ethoxy)-N-(5-fluorothiazol-2-yl)-benzamide

Yield: 20.6%

¹H-NMR (CDCl₃) δ 11.47 (brs, 1H), 7.64 (s, 1H), 7.43 (s, 1H), 7.38-7.29 (m, 2H), 7.23 (s, 1H), 7.15 (d, 1H), 7.10-7.07 (m, 2H), 6.79 (d, 1H), 4.70-4.64 (m, 1H), 3.64-3.52 (m, 2H), 3.43 (s, 3H), 1.36 (d, 3H)

### Example 25

### 3-[trans-2-(2,4-difluorophenyl)vinyl]-5-(2-methoxy-(1S)-methyl-ethoxy)-N-(5-fluorothiazol-2-yl)-benzamide

Yield: 34.3%

¹H-NMR (CDCl₃) δ 11.68 (brs, 1H), 7.63 (s, 1H), 7.53 (q, 1H), 7.42 (s, 1H), 7.32 (s, 1H), 7.19 (d, 1H), 7.05 (d, 1H), 6.86-6.82 (m, 2H), 6.76 (d, 1H), 4.70-4.63 (m, 1H), 3.64-3.52 (m, 2H), 3.43 (s, 3H), 1.36 (d, 3H)

### Example 26

### 3-[trans-2-(2,5-difluorophenyl)vinyl]-5-(2-methoxy-(1S)-methyl-ethoxy)-N-(5-fluorothiazol-2-yl)-benzamide

Yield: 24.0%

¹H-NMR (CDCl₃) δ 11.52 (brs, 1H), 7.65 (s, 1H), 7.43 (t, 1H), 7.33 (d, 1H), 7.28-7.24 (m, 1H), 7.20 (s, 1H), 7.12-6.90 (m, 3H), 6.78 (d, 1H), 4.71-4.63 (m, 1H), 3.64-3.52 (m, 2H), 3.43 (s, 3H), 1.36 (d, 3H)

### Example 27

### 3-[trans-2-(2,3,5-trifluorophenyl)vinyl]-5-(2-methoxy-(1S)-methyl-ethoxy)-N-(5-fluorothiazol-2-yl)-benzamide

Yield: 19.8%

¹H-NMR (CDCl₃) δ 11.05 (brs, 1H), 7.60 (s, 1H), 7.41 (s, 1H), 7.27 (s, 1H), 7.13-7.02 (m, 3H), 6.95 (s, 1H), 6.84 (d, 1H), 4.69-4.63 (m, 1H), 3.64-3.51 (m, 2H), 3.43 (s, 3H), 1.35 (d, 3H)

### Example 28

### 3-[trans-2-(2,3-dimethoxyphenyl)vinyl]-5-(2-methoxy-(1S)-methyl-ethoxy)-N-(5-fluorothiazol-2-yl)-benzamide

Yield: 19.5%

¹H-NMR (CDCl₃) δ 11.25 (brs, 1H), 7.64 (s, 1H), 7.48 (d, 1H), 7.38 (d, 2H), 7.20 (dd, 1H), 7.11-7.05 (m, 2H), 6.87 (dd, 1H), 6.80 (d, 1H), 4.70-4.64 (m, 1H), 3.89 (s, 3H), 3.85 (s, 3H), 3.65-3.52 (m, 2H), 3.43 (s, 3H), 1.36 (d, 3H)

### Example 29

### 3-[trans-2-(2,6-dimethoxyphenyl)vinyl]-5-(2-methoxy-(1S)-methyl-ethoxy)-N-(5-fluorothiazol-2-yl)-benzamide

Yield: 22.8%

¹H-NMR (CDCl₃) δ 10.72 (brs, 1H), 7.62 (s, 1H), 7.52 (d, 2H), 7.45 (t, 1H), 7.36 (dd, 2H), 7.19 (t, 1H), 6.91 (d, 1H), 6.59 (dd, 1H), 4.70-4.64 (m, 1H), 3.90 (s, 6H), 3.64-3.51 (m, 2H), 3.43 (s, 3H), 1.35 (d, 3H)

### Example 30

### 3-[trans-2-(3,4-dimethoxyphenyl)vinyl]-5-(2-methoxy-(1S)-methyl-ethoxy)-N-(5-fluorothiazol-2-yl)-benzamide

Yield: 29.3%

¹H-NMR (CDCl₃) δ 11.46 (brs, 1H), 7.61 (s, 1H), 7.38 (t, 1H), 7.29 (s, 1H), 7.07-7.03 (s, 3H), 6.93-6.86 (m, 2H), 6.81 (d, 1H), 4.70-4.62 (m, 1H), 3.96 (s, 3H), 3.91 (s, 3H), 3.65-3.51 (m, 2H), 3.43 (s, 3H), 1.36 (d, 3H)

### Example 31

### 3-[trans-2-(3,5-dimethoxyphenyl)vinyl]-5-(2-methoxy-(1S)-methyl-ethoxy)-N-(5-fluorothiazol-2-yl)-benzamide

Yield: 39.1%

¹H-NMR (CDCl₃) δ 11.63 (brs, 1H), 7.63 (s, 1H), 7.40 (t, 1H), 7.30 (t, 1H), 7.03 (s, 2H), 6.78 (d, 1H), 6.64 (d, 2H), 6.43 (t, 1H), 4.69-4.62 (m, 1H), 3.84 (s, 6H), 3.64-3.51 (m, 2H), 3.43 (s, 3H), 1.36 (d, 3H)

### Example 32

### 3-[trans-2-(2-nitrophenyl)vinyl]-5-(2-methoxy-(1S)-methyl-ethoxy)-N-(1-methyl-1H-pyrazol-3-yl)-benzamide

3-[(Phosphonic acid diethyl ester)-methyl]-5-(2-methoxy-(1S)-methyl-ethoxy)-N-(1-methyl-1H-pyrazol-3-yl)-benz amide (30.0 mg) prepared in Preparation 5, potassium hydroxide (8.0 mg), and 18-crown-6 (9.0 mg) were dissolved in dichloromethane (1.0 mL).

2-Nitrobenzaldehyde 12.0 mg was added to the solution, which was then stirred at room temperature for 20 hours. The reaction mixture was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/2) to obtain 10.0 mg of the titled compound in the form of yellow liquid (Yield: 33.7%).

¹H-NMR (CDCl₃) δ 8.82 (brs, 1H), 8.00 (dd, 1H), 7.73 (d, 1H), 7.66-7.58 (m, 2H), 7.53 (s, 1H), 7.47-7.41 (m, 2H), 7.31-7.29 (m, 2H), 7.00 (d, 1H), 6.84 (d, 1H), 4.70-4.62 (m, 1H), 3.77 (s, 3H), 3.64-3.51 (m, 2H), 3.43 (s, 3H), 1.35 (d, 3H)

### Examples 33 to 37

The titled compounds of Examples 33 to 37 were prepared, in accordance with the same procedures as in Example 32, using 3-[(phosphonic acid diethyl ester)-methyl]-5-(2-methoxy-(1S)-methyl-ethoxy)-N-(1-methyl-1H-pyrazol-3-yl)-benz amide prepared in Preparation 5; and aldehydes suitable for Examples 33 to 37, instead of 2-nitrobenzaldehyde, respectively.

### Example 33

### 3-[trans-2-(2-fluorophenyl)vinyl]-5-(2-methoxy-(1S)-methyl-ethoxy)-N-(1-methyl-1H-pyrazol-3-yl)-benzamide

Yield: 28.7%

¹H-NMR (CDCl₃) δ 8.55 (brs, 1H), 7.61-7.57 (m, 2H), 7.37 (d, 1H), 7.33-7.23 (m, 4H), 7.18-7.06 (m, 3H), 6.84 (d, 1H), 4.70-4.63 (m, 1H), 3.82 (s, 3H), 3.64-3.52 (m, 2H), 3.43 (s, 3H), 1.35 (d, 3H)

### Example 34

### 3-[trans-2-(2,3-difluorophenyl)vinyl]-5-(2-methoxy-(1S)-methyl-ethoxy)-N-(1-methyl-1H-pyrazol-3-yl)-benzamide

Yield: 31.0%

¹H-NMR (CDCl₃) δ 8.86 (brs, 1H), 7.57 (s, 1H), 7.42-7.25 (m, 3H), 7.22 (s, 1H), 7.16 (s, 1H), 7.12-7.03 (m, 3H), 6.86 (d, 1H), 4.68-4.62 (m, 1H), 3.77 (s, 3H), 3.63-3.51 (m, 2H), 3.43 (s, 3H), 1.35 (d, 3H)

### Example 35

### 3-[trans-2-(2,4-difluorophenyl)vinyl]-5-(2-methoxy-(1S)-methyl-ethoxy)-N-(1-methyl-1H-pyrazol-3-yl)-benzamide

Yield: 31.0%

¹H-NMR (CDCl₃) δ 8.85 (brs, 1H), 7.58-7.51 (m, 2H), 7.38 (d, 1H), 7.29 (d, 1H), 7.24 (d, 1H), 7.20 (d, 1H), 7.04 (d, 1H), 6.91-6.82 (m, 1H), 4.68-4.62 (m, 1H), 3.77 (s, 3H), 3.63-3.51 (m, 2H), 3.43 (s, 3H), 1.34 (d, 3H)

### Example 36

### 3-[trans-2-(2,5-difluorophenyl)vinyl]-5-(2-methoxy-(1S)-methyl-ethoxy)-N-(1-methyl-1H-pyrazol-3-yl)-benzamide

Yield: 10.3%

¹H-NMR (CDCl₃) δ 8.50 (brs, 1H), 7.59 (s, 1H), 7.39 (s, 1H), 7.31-7.23 (m, 2H), 7.13-6.90 (m, 5H), 6.84 (d, 1H), 4.69-4.63 (m, 1H), 3.83 (s, 3H), 3.64-3.51 (m, 2H), 3.43 (s, 3H), 1.35 (d, 3H)

### Example 37

### 3-[trans-2-(2,6-difluorophenyl)vinyl]-5-(2-methoxy-(1S)-methyl-ethoxy)-N-(1-methyl-1H-pyrazol-3-yl)-benzamide

Yield: 10.3%

¹H-NMR (CDCl₃) δ 8.52 (brs, 1H), 7.58 (s, 1H), 7.43-7.38 (m, 2H), 7.31-7.28 (m, 2H), 7.20-7.15 (m, 2H), 6.93 (t, 2H), 6.84 (d, 1H), 4.69-4.64 (m, 1H), 3.83 (s, 3H), 3.64-3.51 (m, 2H), 3.43 (s, 3H), 1.35 (d, 3H)

### Example 38

### 3-[trans-2-(3-nitrophenyl)vinyl]-5-(2-methoxy-(1S)-methyl-ethoxy)-N-(1-methyl-H-pyrazol-3-yl)-benzamide

3-[(phosphonic acid diethyl ester)-methyl]-5-(2-methoxy-(1S)-methyl-ethoxy)-N-(1-methyl-1H-pyrazol-3-yl)-benz amide (30.0 mg) prepared in Preparation 5 was dissolved in tetrahydrofuran (1.0 mL). Potassium tert-butoxide (15.0 mg) and 3-nitrobenzaldehyde (12.0 mg) were added to the solution, which was then stirred at room temperature for 12 hours. The reaction mixture was concentrated under reduced pressure. The resulting residue was dissolved in dichloromethane. The resulting solution was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/2) to obtain 15.0 mg of the titled compound in the form of yellow liquid (Yield: 50.5%).

¹H-NMR (CDCl₃) δ 9.05 (brs, 1H), 8.37 (t, 1H), 8.14-8.11 (dd, 1H), 7.78 (d, 1H), 7.62 (s, 1H), 7.55 (t, 1H), 7.42 (t, 1H), 7.30 (d, 1H), 7.28-7.24 (m, 1H), 7.14 (q, 2H), 6.88 (d, 1H), 4.68-4.61 (m, 1H), 3.75 (s, 3H), 3.64-3.52 (m, 2H), 3.43 (s, 3H), 1.35 (d, 3H)

### Examples 39 to 47

The titled compounds of Examples 39 to 47 were prepared, in accordance with the same procedures as in Example 38, using 3-[(phosphonic acid diethyl ester)-methyl]-5-(2-methoxy-(1S)-methyl-ethoxy)-N-(1-methyl-1H-pyrazol-3-yl)-benz amide prepared in Preparation 5; and aldehydes suitable for Examples 39 to 47, instead of 3-nitrobenzaldehyde, respectively.

### Example 39

### 3-[trans-2-(4-fluorophenyl)vinyl]-5-(2-methoxy-(1S)-methyl-ethoxy)-N-(1-methyl-1H-pyrazol-3-yl)-benzamide

Yield: 57.5%

¹H-NMR (CDCl₃) δ 9.39 (brs, 1H), 7.56 (s, 1H), 7.48-7.44 (m, 2H), 7.38 (t, 1H), 7.27 (s, 1H), 7.20 (t, 1H), 7.09-6.89 (m, 5H), 4.68-4.58 (m, 1H), 3.68 (s, 3H), 3.63-3.50 (m, 2H), 3.43 (s, 3H), 1.34 (d, 3H)

### Example 40

### 3-[trans-2-(3,5-dimethoxyphenyl)vinyl]-5-(2-methoxy-(1S)-methyl-ethoxy)-N-(1-methyl-1H-pyrazol-3-yl)-benzamide

Yield: 58.6%

¹H-NMR (CDCl₃) δ 9.49 (brs, 1H), 7.57 (s, 1H), 7.38 (t, 1H), 7.27 (t, 1H), 7.21 (t, 1H), 6.97 (s, 2H), 6.90 (d, 1H), 6.65 (d, 2H), 6.42 (t, 1H), 4.66-4.59 (m, 1H), 3.85 (s, 6H), 3.66 (s, 3H), 3.63-3.50 (m, 2H), 3.42 (s, 3H), 1.33 (d, 3H)

### Example 41

### 3-[trans-2-(4-methoxyphenyl)vinyl]-5-(2-methoxy-(1S)-methyl-ethoxy)-N-(1-methyl-1H-pyrazol-3-yl)-benzamide

Yield: 69.8%

¹H-NMR (CDCl₃) δ 9.39 (brs, 1H), 7.54 (s, 1H), 7.43 (d, 2H), 7.35 (t, 1H), 7.26 (d, 1H), 7.19 (t, 1H), 7.00 (d, 1H), 6.92-6.85 (m, 4H), 4.67-4.59 (m, 1H), 3.84 (s, 3H), 3.67 (s, 3H), 3.63-3.50 (m, 2H), 3.42 (s, 3H), 1.33 (d, 3H)

### Example 42

### 3-[trans-2-(2-methoxyphenyl)vinyl]-5-(2-methoxy-(1S)-methyl-ethoxy)-N-(1-methyl-1 H-pyrazol-3-yl)-benzamide

Yield: 61.0%

¹H-NMR (CDCl₃) δ 9.47 (s, 1H), 7.56-7.54 (m, 2H), 7.46 (d, 1H), 7.36-7.35 (m, 1H), 7.28-7.24 (m, 3H), 7.02 (d, 1H), 6.97 (t, 1H), 6.92-6.89 (m, 2H), 4.64-4.58 (m, 1H), 3.89 (s, 3H), 3.64 (s, 3H), 3.62-3.49 (m, 2H), 3.42 (s, 3H), 1.33 (d, 3H)

### Example 43

### 3-[trans-2-(2-bromophenyl)vinyl]-5-(2-methoxy-(1S)-methyl-ethoxy)-N-(1-methyl-1H-pyrazol-3-yl)-benzamide

Yield: 59.4%

¹H-NMR (CDCl₃) δ 9.23 (brs, 1H), 7.65-7.54 (m, 3H), 7.47 (d, 1H), 7.39 (t, 1H), 7.34-7.25 (m, 3H), 7.14 (td, 1H), 6.94 (d, 1H), 6.87 (d, 1H), 4.67-4.59 (m, 1H), 3.70 (s, 3H), 3.63-3.50 (m, 2H), 3.43 (s, 3H), 1.34 (d, 3H)

### Example 44

### 3-[trans-2-(3-bromophenyl)vinyl]-5-(2-methoxy-(1S)-methyl-ethoxy)-N-(1-methyl-1H-pyrazol-3-yl)-benzamide

Yield: 62.5%

¹H-NMR (CDCl₃) δ 9.37 (brs, 1H), 7.65 (s, 1H), 7.57 (s, 1H), 7.42-7.37 (m, 3H), 7.28-7.20 (m, 3H), 6.98 (q, 2H), 6.90 (d, 1H), 4.66-4.58 (m, 1H), 3.68 (s, 3H), 3.62-3.50 (m, 2H), 3.42 (s, 3H), 1.34 (d, 3H)

### Example 45

### 3-[trans-2-(4-bromophenyl)vinyl]-5-(2-methoxy-(1S)-methyl-ethoxy)-N-(1-methyl-1H-pyrazol-3-yl)-benzamide

Yield: 56.3%

¹H-NMR (CDCl₃) δ 9.44 (brs, 1H), 7.57 (s, 1H), 7.49 (dd, 2H), 7.39 (t, 1H), 7.35 (dd, 2H), 7.26 (d, 1H), 7.20 (t, 1H), 6.98 (q, 2H), 6.90 (d, 1H), 4.67-4.58 (m, 1H), 3.67 (s, 3H), 3.62-3.50 (m, 2H), 3.42 (s, 3H), 1.33 (d, 3H)

### Example 46

### 3-[trans-2-(2-chlorophenyl)vinyl]-5-(2-methoxy-(1S)-methyl-ethoxy)-N-(1-methyl-1H-pyrazol-3-yl)-benzamide

Yield: 65.6%

¹H-NMR (CDCl₃) δ 9.19 (brs, 1H), 7.65 (dd, 1H), 7.56 (s, 1H), 7.52 (d, 1H), 7.41-7.38 (m, 2H), 7.30-7.19 (m, 4H), 6.99 (d, 1H), 6.87 (d, 1H), 4.67-4.59 (m, 1H), 3.70 (s, 3H), 3.63-3.50 (m, 2H), 3.43 (s, 3H), 1.34 (d, 3H)

### Example 47

### 3-[trans-2-(3-chlorophenyl)vinyl]-5-(2-methoxy-(1S)-methyl-ethoxy)-N-(1-methyl-1H-pyrazol-3-yl)-benzamide

Yield: 62.1%

¹H-NMR (CDCl₃) δ 9.32 (brs, 1H), 7.57 (s, 1H), 7.49 (s, 1H), 7.40-7.20 (m, 6H), 7.00 (q, 2H), 6.90 (d, 1H), 4.67-4.59 (m, 1H), 3.69 (s, 3H), 3.62-3.50 (m, 2H), 3.43 (s, 3H), 1.34 (d, 3H)

### Example 48

### 3-[trans-2-(2-methoxyphenyl)vinyl]-5-(2-methoxy-(1S)-methyl-ethoxy)-N-(pyrazin-2-yl)-benzamide

3-[(Phosphonic acid diethyl ester)-methyl]-5-(2-methoxy-(1S)-methyl-ethoxy)-N-(pyrazin-2-yl)-benzamide (30.0 mg) prepared in Preparation 6 was dissolved in tetrahydrofuran (1.0 mL). Potassium tert-butoxide (15.0 mg) and 2-methoxybenzaldehyde (9.0 μL) were added to the solution, which was then stirred at room temperature for 12 hours. The reaction mixture was concentrated under reduced pressure. The resulting residue was dissolved in dichloromethane. The solution was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/2) to obtain 19.0 mg of the titled compound in the form of yellow liquid (Yield: 65.6%).

¹H-NMR (CDCl₃) δ 9.73 (s, 1H), 8.58 (brs, 1H), 8.39 (d, 1H), 8.29 (dd, 1H), 7.63-7.52 (m, 3H), 7.39 (t, 1H), 7.32-7.26 (m, 2H), 7.11 (d, 1H), 6.98 (t, 1H), 6.92 (d, 1H), 4.74-4.65 (m, 1H), 3.91 (s, 3H), 3.65-3.52 (m, 2H), 3.44 (s, 3H), 1.37 (d, 3H)

### Examples 49 to 60

The titled compounds of Examples 49 to 60 were prepared, in accordance with the same procedures as in Example 48, using 3-[(phosphonic acid diethyl ester)-methyl]-5-(2-methoxy-(1S)-methyl-ethoxy)-N-(pyrazin-2-yl)-benzamide prepared in Preparation 6; and aldehydes suitable for Examples 49 to 60, instead of 2-methoxybenzaldehyde, respectively.

### Example 49

### 3-[trans-2-(4-methoxyphenyl)vinyl]-5-(2-methoxy-(1S)-methyl-ethoxy)-N-(pyrazin-2-yl)-benzamide

Yield: 62.2%

¹H-NMR (CDCl₃) δ 9.73 (d, 1H), 8.56 (brs, 1H), 8.39 (d, 1H), 8.28 (dd, 1H), 7.59 (s, 1H), 7.46 (d, 2H), 7.37 (t, 1H), 7.26 (s, 1H), 7.13 (d, 1H), 6.97-6.90 (m, 3H), 4.73-4.64 (m, 1H), 3.84 (s, 3H), 3.65-3.52 (m, 2H), 3.44 (s, 3H), 1.36 (d, 3H)

### Example 50

### 3-[trans-2-(3,5-dimethoxyphenyl)vinyl]-5-(2-methoxy-(1S)-methyl-ethoxy)-N-(pyrazin-2-yl)-benzamide

Yield: 64.5%

¹H-NMR (CDCl₃) δ 9.73 (d, 1H), 8.54 (brs, 1H), 8.40 (d, 1H), 8.29 (dd, 1H), 7.61 (s, 1H), 7.40 (d, 1H), 7.28 (s, 1H), 7.08 (q, 2H), 6.68 (d, 2H), 6.43 (t, 1H), 4.73-4.65 (m, 1H), 3.84 (s, 6H), 3.65-3.52 (m, 2H), 3.44 (s, 3H), 1.37 (d, 3H)

### Example 51

### 3-[trans-2-(2-fluorophenyl)vinyl]-5-(2-methoxy-(1S)-methyl-ethoxy)-N-(pyrazin-2-yl)-benzamide

Yield: 60.5%

¹H-NMR (CDCl₃) δ 9.75 (s, 1H), 8.59 (brs, 1H), 8.45 (d, 1H), 8.32 (d, 1H), 7.62-7.57 (m, 2H), 7.42 (t, 1H), 7.35-7.23 (m, 3H), 7.19-7.06 (m, 3H), 4.74-4.66 (m, 1H), 3.65-3.53 (m, 2H), 3.44 (s, 3H), 1.37 (d, 3H)

### Example 52

### 3-[trans-2-(4-fluorophenyl)vinyl]-5-(2-methoxy-(1S)-methyl-ethoxy)-N-(pyrazin-2-yl)-benzamide

Yield: 67.6%

¹H-NMR (CDCl₃) δ 9.73 (s, 1H), 8.58 (brs, 1H), 8.40 (d, 1H), 8.29 (dd, 1H), 7.61 (t, 1H), 7.51-7.47 (m, 2H), 7.39 (d, 1H), 7.27 (s, 1H), 7.16-6.97 (m, 4H), 4.73-4.65 (m, 1H), 3.65-3.52 (m, 2H), 3.44 (s, 3H), 1.37 (d, 3H)

### Example 53

### 3-[trans-2-(2-chlorophenyl)vinyl]-5-(2-methoxy-(1S)-methyl-ethoxy)-N-(pyrazin-2-yl)-benzamide

Yield: 68.4%

¹H-NMR (CDCl₃) δ 9.75 (s, 1H), 8.57 (brs, 1H), 8.40 (d, 1H), 8.29 (t, 1H), 7.68 (dd, 1H), 7.63 (s, 1H), 7.57 (d, 1H), 7.43-7.39 (m, 2H), 7.35 (s, 1H), 7.31-7.20 (m, 2H), 7.06 (d, 1H), 4.74-4.65 (m, 1H), 3.65-3.53 (m, 2H), 3.44 (s, 3H), 1.37 (d, 3H)

### Example 54

### 3-[trans-2-(3-chlorophenyl)vinyl]-5-(2-methoxy-(1S)-methyl-ethoxy)-N-(pyrazin-2-yl)-benzamide

Yield: 61.5%

¹H-NMR (CDCl₃) δ 9.75 (s, 1H), 8.55 (brs, 1H), 8.42 (s, 1H), 8.29 (dd, 1H), 7.62 (t, 1H), 7.51 (t, 1H), 7.43-7.37 (m, 2H), 7.33-7.24 (m, 3H), 7.10 (q, 2H), 4.73-4.65 (m, 1H), 3.65-3.53 (m, 2H), 3.44 (s, 3H), 1.37 (d, 3H)

### Example 55

### 3-[trans-2-(2,3-difluorophenyl)vinyl]-5-(2-methoxy-(1S)-methyl-ethoxy)-N-(pyrazin-2-yl)-benzamide

Yield: 68.1%

¹H-NMR (CDCl₃) δ 9.72 (d, 1H), 8.52 (brs, 1H), 8.41 (d, 1H), 8.30 (dd, 1H), 7.63 (s, 1H), 7.44 (t, 1H), 7.37-7.31 (m, 2H), 7.28 (s, 1H), 7.19 (d, 1H), 7.11-7.07 (m, 2H), 4.74-4.66 (m, 1H), 3.65-3.53 (m, 2H), 3.44 (s, 3H), 1.37 (d, 3H)

### Example 56

### 3-[trans-2-(2,4-difluorophenyl)vinyl]-5-(2-methoxy-(1S)-methyl-ethoxy)-N-(pyrazin-2-yl)-benzamide

Yield: 64.7%

¹H-NMR (CDCl₃) δ 9.72 (d, 1H), 8.55 (brs, 1H), 8.40 (d, 1H), 8.29 (dd, 1H), 7.62-7.50 (m, 2H), 7.42 (t, 1H), 7.30 (d, 1H), 7.25 (d, 1H), 7.09 (d, 1H), 6.94-6.83 (m, 2H), 4.73-4.65 (m, 1H), 3.65-3.53 (m, 2H), 3.44 (s, 3H), 1.37 (d, 3H)

### Example 57

### 3-[trans-2-(2,5-difluorophenyl)vinyl]-5-(2-methoxy-(1S)-methyl-ethoxy)-N-(pyrazin-2-yl)-benzamide

Yield: 71.5%

¹H-NMR (CDCl₃) δ 9.72 (d, 1H), 8.52 (brs, 1H), 8.41 (d, 1H), 8.30 (t, 1H), 7.63 (s, 1H), 7.44 (t, 1H), 7.32-7.25 (m, 3H), 7.14 (d, 1H), 7.09-6.92 (m, 2H), 4.72-4.67 (m, 1H), 3.65-3.53 (m, 2H), 3.44 (s, 3H), 1.37 (d, 3H)

### Example 58

### 3-[trans-2-(2,6-difluorophenyl)vinyl]-5-(2-methoxy-(1S)-methyl-ethoxy)-N-(pyrazin-2-yl)-benzamide

Yield: 61.3%

¹H-NMR (CDCl₃) δ 9.72 (d, 1H), 8.49 (brs, 1H), 8.40 (d, 1H), 8.30 (dd, 1H), 7.62 (s, 1H), 7.46-7.41 (m, 2H), 7.34 (t, 1H), 7.22-7.18 (m, 2H), 6.94 (t, 2H), 4.74-4.66 (m, 1H), 3.65-3.53 (m, 2H), 3.44 (s, 3H), 1.37 (d, 3H)

### Example 59

### 3-[trans-2-(3-bromophenyl)vinyl]-5-(2-methoxy-(1S)-methyl-ethoxy)-N-(pyrazin-2-yl)-benzamide

Yield: 52.6%

¹H-NMR (CDCl₃) δ 9.72 (d, 1H), 8.53 (brs, 1H), 8.40 (d, 1H), 8.29 (dd, 1H), 7.68 (s, 1H), 7.62 (s, 1H), 7.44-7.40 (m, 3H), 7.29-7.22 (m, 2H), 7.09 (s, 2H), 4.73-4.65 (m, 1H), 3.65-3.53 (m, 2H), 3.44 (s, 3H), 1.37 (d, 3H)

### Example 60

### 3-[trans-2-(4-bromophenyl)vinyl]-5-(2-methoxy-(1S)-methyl-ethoxy)-N-(pyrazin-2-yl)-benzamide

Yield: 55.7%

¹H-NMR (CDCl₃) δ 9.72 (d, 1H), 8.53 (brs, 1H), 8.40 (d, 1H), 8.29 (dd, 1H), 7.61 (s, 1H), 7.50 (d, 2H), 7.41-7.37 (m, 3H), 7.28 (s, 1H), 7.09 (q, 2H), 4.73-4.65 (m, 1H), 3.65-3.52 (m, 2H), 3.44 (s, 3H), 1.37 (d, 3H)

### Example 61

### 3-[trans-2-(2-methoxyphenyl)vinyl]-5-(2-methoxy-(1S)-methyl-ethoxy)-N-(nicotinic acid-3-methyl ester-6-yl)-benzamide

3-[(Phosphonic acid diethyl ester)-methyl]-5-(2-methoxy-(1S)-methyl-ethoxy)-N-(nicotinic acid-3-methyl ester-6-yl)-benzamide (100.0 mg) prepared in Preparation 7 was dissolved in tetrahydrofuran (10.0 mL). Potassium tert-butoxide (45.0 mg) and 2-methoxybenzaldehyde (30.0 μL) were added to the solution, which was then stirred at room temperature for 12 hours. The reaction mixture was concentrated under reduced pressure. The resulting residue was dissolved in dichloromethane. The solution was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/1) to obtain 9.0 mg of the titled compound in the form of yellow liquid (Yield: 9.3%).

¹H-NMR (CDCl₃) δ 8.95 (s, 1H), 8.80 (s, 1H), 8.48 (d, 1H), 8.37 (dd, 1H), 7.62-7.51 (m, 3H), 7.38 (s, 1H), 7.32-7.26 (m, 2H), 7.10 (d, 1H), 6.99 (t, 1H), 6.92 (d, 1H), 4.73-4.65 (m, 1H), 3.95 (s, 3H), 3.91 (s, 3H), 3.65-3.52 (m, 2H), 3.44 (s, 3H), 1.36 (d, 3H)

### Examples 62 to 69

The titled compounds of Examples 62 to 69 were prepared, in accordance with the same procedures as in Example 61, using 3-[(phosphonic acid diethyl ester)-methyl]-5-(2-methoxy-(1S)-methyl-ethoxy)-N-(nicotinic acid-3-methyl ester-6-yl)-benzamide prepared in Preparation 7; and aldehydes suitable for Examples 62 to 69, instead of 2-methoxybenzaldehyde, respectively.

### Example 62

### 3-[trans-2-(2-nitrophenyl)vinyl]-5-(2-methoxy-(1S)-methyl-ethoxy)-N-(nicotinic acid-3-methyl ester-6-yl)-benzamide

Yield: 20.3%

¹H-NMR (CDCl₃) δ 8.98 (s, 1H), 8.87 (s, 1H), 8.47-8.34 (m, 2H), 8.01 (d, 1H), 7.75 (d, 1H), 7.68-7.60 (m, 3H), 7.48-7.44 (m, 2H), 7.36 (s, 1H), 7.06 (d, 1H), 4.73-4.68 (m, 1H), 3.96 (s, 3H), 3.65-3.53 (m, 2H), 3.45 (s, 3H), 1.37 (d, 3H)

### Example 63

### 3-[trans-2-(3-nitrophenyl)vinyl]-5-(2-methoxy-(1S)-methyl-ethoxy)-N-(nicotinic acid-3-methyl ester-6-yl)-benzamide

Yield: 40.7%

¹H-NMR (CDCl₃) δ 9.01 (s, 1H), 8.91 (s, 1H), 8.48 (d, 1H), 8.38 (m, 1H), 7.80 (d, 1H), 7.71-7.20 (m, 8H), 4.73-4.68 (m, 1H), 3.95 (s, 3H), 3.66-3.53 (m, 2H), 3.44 (s, 3H), 1.37 (d, 3H)

### Example 64

### 3-[trans-2-(2-fluorophenyl)vinyl]-5-(2-methoxy-(1S)-methyl-ethoxy)-N-(nicotinic acid-3-methyl ester-6-yl)-benzamide

Yield: 12.9%

¹H-NMR (CDCl₃) δ 8.95 (s, 1H), 8.79 (s, 1H), 8.48 (d, 1H), 8.37 (d, 1H), 7.63-7.58 (m, 2H), 7.42 (s, 1H), 7.35-7.07 (m, 6H), 4.72-4.68 (m, 1H), 3.95 (s, 3H), 3.65-3.53 (m, 2H), 3.44 (s, 3H), 1.37 (d, 3H)

### Example 65

### 3-[trans-2-(4-fluorophenyl)vinyl]-5-(2-methoxy-(1S)-methyl-ethoxy)-N-(nicotinic acid-3-methyl ester-6-yl)-benzamide

Yield: 12.9%

¹H-NMR (CDCl₃) δ 8.96 (s, 1H), 8.80 (s, 1H), 8.48 (d, 1H), 8.37 (d, 1H), 7.60 (s, 1H), 7.51-7.47 (m, 2H), 7.40 (s, 1H), 7.36-7.32 (m, 1H), 7.16-6.97 (m, 4H), 4.72-4.68 (m, 1H), 3.95 (s, 3H), 3.65-3.52 (m, 2H), 3.44 (s, 3H), 1.36 (d, 3H)

### Example 66

### 3-[trans-2-(2,3-difluorophenyl)vinyl]-5-(2-methoxy-(1S)-methyl-ethoxy)-N-(nicotinic acid-3-methyl ester-6-yl)-benzamide

Yield: 26.9%

¹H-NMR (CDCl₃) δ 8.95 (s, 1H), 8.82 (s, 1H), 8.47 (d, 1H), 8.37 (dd, 1H), 7.62 (s, 1H), 7.43 (t, 1H), 7.36-7.31 (m, 2H), 7.27-7.26 (m, 1H), 7.18 (d, 1H), 7.11-7.07 (m, 2H), 4.72-4.68 (m, 1H), 3.95 (s, 3H), 3.65-3.53 (m, 2H), 3.44 (s, 3H), 1.37 (d, 3H)

### Example 67

### 3-[trans-2-(2,4-difluorophenyl)vinyl]-5-(2-methoxy-(1S)-methyl-ethoxy)-N-(nicotinic acid-3-methyl ester-6-yl)-benzamide

Yield: 10.4%

¹H-NMR (CDCl₃) δ 8.95 (d, 1H), 8.79 (s, 1H), 8.47 (d, 1H), 8.37 (dd, 1H), 7.60-7.54 (m, 2H), 7.41 (t, 1H), 7.30 (s, 1H), 7.23 (s, 1H), 7.09 (d, 1H), 6.92-6.83 (m, 2H), 4.72-4.67 (m, 1H), 3.95 (s, 3H), 3.65-3.53 (m, 2H), 3.44 (s, 3H), 1.36 (d, 3H)

### Example 68

### 3-[trans-2-(2,5-difluorophenyl)vinyl]-5-(2-methoxy-(1S)-methyl-ethoxy)-N-(nicotinic acid-3-methyl ester-6-yl)-benzamide

Yield: 22.8%

¹H-NMR (CDCl₃) δ 8.94 (s, 1H), 8.85 (s, 1H), 8.47 (dd, 1H), 8.37 (dd, 1H), 7.62 (s, 1H), 7.43 (t, 1H), 7.30-7.24 (m, 3H), 7.12 (d, 1H), 7.06-7.02 (m, 1H), 6.97-6.94 (m, 1H), 4.72-4.67 (m, 1H), 3.95 (s, 3H), 3.65-3.53 (m, 2H), 3.44 (s, 3H), 1.36 (d, 3H)

### Example 69

### 3-[trans-2-(2,6-difluorophenyl)vinyl]-5-(2-methoxy-(1S)-methyl-ethoxy)-N-(nicotinic acid-3-methyl ester-6-yl)-benzamide

Yield: 20.7%

¹H-NMR (CDCl₃) δ 8.95 (d, 1H), 8.79 (s, 1H), 8.47 (dd, 1H), 8.37 (dd, 1H), 7.61 (s, 1H), 7.45-7.41 (m, 2H), 7.33 (s, 1H), 7.22-7.17 (m, 2H), 6.96-6.91 (m, 2H), 4.72-4.67 (m, 1H), 3.95 (s, 3H), 3.65-3.53 (m, 2H), 3.44 (s, 3H), 1.37 (d, 3H)

### Example 70

### 3-[trans-2-(2-fluorophenyl)vinyl]-5-isobutoxy-N-(thiazol-2-yl)-benzamide

3-(Triphenylphosphonium bromide-methyl)-5-isobutoxy-N-(thiazol-2-yl)-benzamide (100.0 mg) prepared in Preparation 2, potassium hydroxide (30.0 mg), and 18-crown-6 (7.0 mg) were dissolved in dichloromethane (2.0 mL). 2-Fluorobenzaldehyde (31.0 μL) was added to the solution, which was then stirred at room temperature for 12 hours. The reaction mixture was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=5/1) to obtain 50.0 mg of a compound in the form of yellow liquid.

The obtained compound (50.0 mg) was dissolved in heptane (2.0 mL). A piece of iodine was added to the solution, which was then stirred at 100° C. for 16 hours. Ethyl acetate was added to the reaction mixture, which was washed with a saturated sodium hydrogen sulfate solution and brine three times, dried on anhydrous magnesium sulfate, and then concentrated under reduced pressure to obtain 25.0 mg of the titled compound in the form of yellow liquid (Yield: 24.3%).

¹H-NMR (CDCl₃) δ 12.83 (brs, 1H), 7.73 (s, 1H), 7.57-6.92 (m, 10H), 3.79 (d, 2H), 2.13-2.05 (m, 1H), 1.04 (d, 6H)

### Examples 71 and 72

The titled compounds of Examples 71 and 72 were prepared, in accordance with the same procedures as in Example 70, using 3-(triphenylphosphonium bromide-methyl)-5-isobutoxy-N-(thiazol-2-yl)-benzamide prepared in Preparation 2; and 2,5-difluorobenzaldehyde and 2,6-difluorobenzaldehyde, instead of 2-fluorobenzaldehyde, respectively.

### Example 71

### 3-[trans-2-(2,5-difluorophenyl)vinyl]-5-isobutoxy-N-(thiazol-2-yl)-benzamide

Yield: 27.8%

¹H-NMR (CDCl₃) δ 12.62 (brs, 1H), 7.74 (s, 1H), 7.48 (d, 1H), 7.29-6.89 (m, 8H), 3.79 (d, 2H), 2.18-2.09 (m, 1H), 1.05 (d, 6H)

### Example 72

### 3-[trans-2-(2,6-difluorophenyl)vinyl]-5-isobutoxy-N-(thiazol-2-yl)-benzamide

Yield: 18.6%

¹H-NMR (CDCl₃) δ 12.67 (brs, 1H), 7.74 (s, 1H), 7.47 (s, 1H), 7.39 (d, 1H), 7.30 (s, 1H), 7.18-7.10 (m, 3H), 6.94-6.89 (m, 3H), 3.79 (d, 2H), 2.20-2.10 (m, 1H), 1.05 (d, 6H)

### Example 73

### 3-[trans-2-(2-methoxyphenyl)vinyl]-5-(2-methoxy-(1S)-methyl-ethoxy)-N-(nicotinic acid-6-yl)-benzamide

The compound prepared in Example 61 (7.0 mg) was dissolved in tetrahydrofuran (0.5 mL). A 3 N sodium hydroxide solution (0.5 mL) was added to the solution at room temperature for 12 hours. The reaction mixture was concentrated under reduced pressure and then neutralized with a 1 N hydrochloric acid solution to obtain a white precipitate. The precipitate was washed with distilled water and then dried under reduced pressure to obtain 4.0 mg of the titled compound in the form of white solid (Yield: 57.7%).

¹H-NMR (d₆-DMSO) δ 10.88 (s, 1H), 8.78 (s, 1H), 8.17-8.09 (m, 2H), 7.86 (s, 1H), 7.67 (d, 1H), 7.57-7.50 (m, 2H), 7.31-7.23 (m, 3H), 7.07-6.97 (m, 2H), 4.84-4.79 (m, 1H), 3.88 (s, 3H), 3.54-3.48 (m, 2H), 3.32 (s, 3H), 1.27 (d, 3H)

### Examples 74 to 81

The titled compounds of Examples 74 to 81 were prepared, in accordance with the same procedures as in Example 73, using the compounds prepared in Examples 62 to 69, instead of the compound prepared in Example 61, respectively.

### Example 74

### 3-[trans-2-(2-nitrophenyl)vinyl]-5-(2-methoxy-(1S)-methyl-ethoxy)-N-(nicotinic acid-6-yl)-benzamide

Yield: 52.4%

¹H-NMR (d₆-DMSO) δ 8.89 (s, 1H), 8.31 (s, 2H), 8.02-7.94 (m, 2H), 7.88 (s, 1H), 7.79-7.75 (m, 1H), 7.63-7.55 (m, 3H), 7.42-7.31 (m, 2H), 4.86-4.80 (m, 1H), 3.54-3.49 (m, 2H), 3.31 (s, 3H), 1.27 (d, 3H)

### Example 75

### 3-[trans-2-(3-nitrophenyl)vinyl]-5-(2-methoxy-(1S)-methyl-ethoxy)-N-(nicotinic acid-6-yl)-benzamide

Yield: 36.7%

¹H-NMR (d₆-DMSO) δ 11.21 (s, 1H), 8.91 (s, 1H), 8.46 (s, 1H), 8.34 (s, 2H), 8.14-8.08 (m, 2H), 7.93 (s, 1H), 7.70 (t, 1H), 7.58-7.46 (m, 4H), 4.85-4.81 (m, 1H), 3.55-3.49 (m, 2H), 3.32 (s, 3H), 1.28 (d, 3H)

### Example 76

### 3-[trans-2-(2-fluorophenyl)vinyl]-5-(2-methoxy-(1S)-methyl-ethoxy)-N-(nicotinic acid-6-yl)-benzamide

Yield: 88.8%

¹H-NMR (d₆-DMSO) δ 11.28 (s, 1H), 8.91 (s, 1H), 8.34 (s, 2H), 7.94 (s, 1H), 7.81 (t, 1H), 7.53-7.23 (m, 7H), 4.85-4.80 (m, 1H), 3.55-3.48 (m, 2H), 3.32 (s, 3H), 1.27 (d, 3H)

### Example 77

### 3-[trans-2-(4-fluorophenyl)vinyl]-5-(2-methoxy-(1S)-methyl-ethoxy)-N-(nicotinic acid-6-yl)-benzamide

Yield: 88.8%

¹H-NMR (d₆-DMSO) δ 11.21 (s, 1H), 8.90 (s, 1H), 8.33 (s, 2H), 7.87 (s, 1H), 7.72-7.66 (m, 2H), 7.51 (s, 1H), 7.44-7.20 (m, 5H), 4.85-4.80 (m, 1H), 3.55-3.48 (m, 2H), 3.30 (s, 3H), 1.27 (d, 3H)

### Example 78

### 3-[trans-2-(2,3-difluorophenyl)vinyl]-5-(2-methoxy-(1S)-methyl-ethoxy)-N-(nicotinic acid-6-yl)-benzamide

Yield: 62.8%

¹H-NMR (d₆-DMSO) δ 11.25 (s, 1H), 8.91 (s, 1H), 8.34 (s, 2H), 7.96 (s, 1H), 7.65-7.25 (m, 7H), 4.86-4.81 (m, 1H), 3.55-3.49 (m, 2H), 3.32 (s, 3H), 1.28 (d, 3H)

### Example 79

### 3-[trans-2-(2,4-difluorophenyl)vinyl]-5-(2-methoxy-(1S)-methyl-ethoxy)-N-(nicotinic acid-6-yl)-benzamide

Yield: 80.0%

¹H-NMR (d₆-DMSO) δ 11.23 (s, 1H), 8.90 (s, 1H), 8.33 (s, 2H), 7.92-7.84 (m, 2H), 7.53-7.15 (m, 6H), 4.86-4.81 (m, 1H), 3.54-3.48 (m, 2H), 3.32 (s, 3H), 1.27 (d, 3H)

### Example 80

### 3-[trans-2-(2,5-difluorophenyl)vinyl]-5-(2-methoxy-(1S)-methyl-ethoxy)-N-(nicotinic acid-6-yl)-benzamide

Yield: 62.8%

¹H-NMR (d₆-DMSO) δ 11.29 (s, 1H), 8.94 (s, 1H), 8.36 (s, 2H), 7.97 (s, 1H), 7.75-7.69 (m, 1H), 7.58-7.19 (m, 6H), 4.87-4.82 (m, 1H), 3.56-3.50 (m, 2H), 3.35 (s, 3H), 1.30 (d, 3H)

### Example 81

### 3-[trans-2-(2,6-difluorophenyl)vinyl]-5-(2-methoxy-(1S)-methyl-ethoxy)-N-(nicotinic acid-6-yl)-benzamide

Yield: 71.2%

¹H-NMR (d₆-DMSO) δ 11.28 (s, 1H), 8.91 (d, 1H), 8.35 (s, 2H), 7.95 (s, 1H), 7.54-7.16 (m, 7H), 4.87-4.82 (m, 1H), 3.57-3.46 (m, 2H), 3.32 (s, 3H), 1.27 (d, 3H)

### Example 82

### 3-[trans-2-(2-methoxyphenyl)vinyl]-5-(2-methoxy-(1S)-methyl-ethoxy)-N-(5-fluorothiazol-2-yl)-benzamide

3-[trans-2-(2-Methoxyphenyl)vinyl]-5-(2-methoxy-(1S)-methyl-ethoxy)-benzoic acid (50.0 mg) prepared in Preparation 8, HOBT (41.0 mg), EDAC (58.0 mg), triethylamine (42.0 μL), and 2-amino-5-fluorothiazole (23.0 mg) were dissolved in dichloromethane (5.0 mL) and then stirred at room temperature for 12 hours. The reaction mixture was washed with a 1 N hydrochloric acid solution, a saturated sodium hydrogen carbonate solution, distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/1) to obtain 5.0 mg of the titled compound in the form of yellow liquid (Yield: 6.9%).

¹H-NMR (CDCl₃) δ 7.56 (dd, 1H), 7.31 (d, 1H), 7.27-7.23 (m, 2H), 7.13-7.11 (m, 2H), 7.04 (d, 1H), 6.99-6.89 (m, 2H), 6.80 (dd, 1H), 4.63-4.56 (m, 1H), 3.89 (s, 3H), 3.62-3.48 (m, 2H), 3.42 (s, 3H), 1.33 (d, 3H)

### Example 83

### 3-[trans-2-(2-methoxyphenyl)vinyl]-5-(2-methoxy-(1S)-methyl-ethoxy)-N-(1-methyl-1H-pyrazol-3-yl)-benzamide

The titled compound was prepared, in accordance with the same procedures as in Example 82, using 3-[trans-2-(2-methoxyphenyl)vinyl]-5-(2-methoxy-(1S)-methyl-ethoxy)-benzoic acid prepared in Preparation 8; and 1-methyl-3-amino-1H-pyrazole, instead of 2-amino-5-fluorothiazole.

Yield: 40.6%

¹H-NMR (CDCl₃) δ 9.47 (s, 1H), 7.56-7.54 (m, 2H), 7.46 (d, 1H), 7.36-7.35 (m, 1H), 7.28-7.24 (m, 3H), 7.02 (d, 1H), 6.97 (t, 1H), 6.92-6.89 (m, 2H), 4.64-4.58 (m, 1H), 3.89 (s, 3H), 3.64 (s, 3H), 3.62-3.49 (m, 2H), 3.42 (s, 3H), 1.33 (d, 3H)

### Example 84

### 3-[2-(2-fluorophenyl)ethyl]-5-(2-methoxy-(1S)-methyl-ethoxy)-N-(thiazol-2-yl)-benzamide

The compound prepared in Example 6 (27.0 mg) was dissolved in ethyl acetate (2.0 mL) and then Pd/C (10.0 mg) was added thereto. Hydrogenation reaction was performed by stirring the solution at room temperature for 12 hours, using a hydrogen balloon. The reaction mixture was filtered with celite pad. The filtrate was concentrated under reduced pressure to obtain 23.0 mg of the titled compound in the form of yellow liquid (Yield: 71.0%).

¹H-NMR (CDCl₃) δ 7.42 (s, 1H), 7.38-7.35 (m, 2H), 7.28-6.99 (m, 6H), 4.63-4.57 (m, 1H), 3.59-3.47 (m, 2H), 3.41 (s, 3H), 2.95-2.91 (m, 4H), 1.30 (d, 3H)

### Examples 85 to 92

The titled compounds of Examples 85 to 92 were prepared, in accordance with the same procedures as in Example 84, using the compound prepared in Examples 10 to 17, instead of the compound prepared in Example 6, respectively.

### Example 85

### 3-[2-(2,4-difluorophenyl)ethyl]-5-(2-methoxy-(1S)-methyl-ethoxy)-N-(thiazol-2-yl)-benzamide

Yield: 72.3%

¹H-NMR (CDCl₃) δ 7.44-7.41 (m, 1H), 7.27-7.19 (m, 2H), 7.08-6.95 (m, 3H), 6.79-6.75 (m, 2H), 4.61-4.52 (m, 1H), 3.62-3.49 (m, 2H), 3.41 (s, 3H), 2.94-2.90 (m, 4H), 1.30 (d, 3H)

### Example 86

### 3-[2-(2,5-difluorophenyl)ethyl]-5-(2-methoxy-(1S)-methyl-ethoxy)-N-(thiazol-2-yl)-benzamide

Yield: 72.3%

¹H-NMR (CDCl₃) δ 7.43-7.40 (m, 1H), 7.31 (d, 1H), 7.00-6.80 (m, 6H), 4.63-4.57 (m, 1H), 3.62-3.49 (m, 2H), 3.41 (s, 3H), 2.95-2.89 (m, 4H), 1.31 (d, 3H)

### Example 87

### 3-[2-(2,6-difluorophenyl)ethyl]-5-(2-methoxy-(1S)-methyl-ethoxy)-N-(thiazol-2-yl)-benzamide

Yield: 72.3%

¹H-NMR (CDCl₃) δ 7.42 (d, 1H), 7.38 (s, 1H), 7.32 (d, 1H), 7.20-7.10 (m, 1H), 7.02-6.98 (m, 2H), 6.84 (t, 2H), 4.63-4.57 (m, 1H), 3.60-3.45 (m, 2H), 3.41 (s, 3H), 2.99-2.88 (m, 4H), 1.31 (d, 3H)

### Example 88

### 3-[2-(2-aminophenyl)ethyl]-5-(2-methoxy-(1S)-methyl-ethoxy)-N-(thiazol-2-yl)-benzamide

Yield: 75.9%

¹H-NMR (CDCl₃) δ 7.42-7.35 (m, 3H), 7.10-6.98 (m, 4H), 6.74-6.68 (m, 2H), 4.63-4.57 (m, 1H), 3.63-3.50 (m, 2H), 3.41 (s, 3H), 3.00-2.80 (m, 4H), 1.31 (d, 3H)

### Example 89

### 3-[2-(2-methoxyphenyl)ethyl]-5-(2-methoxy-(1S)-methyl-ethoxy)-N-(thiazol-2-yl)-benzamide

Yield: 43.9%

¹H-NMR (CDCl₃) δ 7.42-7.20 (m, 4H), 7.08-6.85 (m, 5H), 4.62-4.52 (m, 1H), 3.83 (s, 3H), 3.62-3.48 (m, 2H), 3.42 (s, 3H), 2.93-2.88 (m, 4H), 1.31 (d, 3H)

### Example 90

### 3-[2-(2,3-dimethoxyphenyl)ethyl]-5-(2-methoxy-(1S)-methyl-ethoxy)-N-(thiazol-2-yl)-benzamide

Yield: 43.9%

¹H-NMR (CDCl₃) δ 7.47-7.24 (m, 2H), 7.10-6.70 (m, 6H), 4.63-4.53 (m, 1H), 3.85 (s, 3H), 3.82 (s, 3H), 3.60-3.47 (m, 2H), 3.41 (s, 3H), 2.94-2.90 (m, 4H), 1.30 (d, 3H)

### Example 91

### 3-[2-(3-aminophenyl)ethyl]-5-(2-methoxy-(1S)-methyl-ethoxy)-N-(thiazol-2-yl)-benzamide

Yield: 71.9%

¹H-NMR (CDCl₃) δ 12.42 (brs, 1H), 7.43-7.37 (m, 2H), 7.11-6.93 (m, 4H), 6.56-6.47 (m, 3H), 4.60-4.53 (m, 1H), 3.59-3.46 (m, 2H), 3.40 (s, 3H), 2.88-2.79 (m, 4H), 1.29 (d, 3H)

### Example 92

### 3-[2-(4-aminophenyl)ethyl]-5-(2-methoxy-(1S)-methyl-ethoxy)-N-(thiazol-2-yl)-benzamide

Yield: 79.9%

¹H-NMR (CDCl₃) δ 12.47 (brs, 1H), 7.42 (s, 1H), 7.32 (s, 1H), 7.09 (d, 1H), 6.98 (s, 1H), 6.93-6.89 (m, 3H), 6.60 (d, 2H), 4.59-4.53 (m, 1H), 3.59-3.46 (m, 2H), 3.40 (s, 3H), 2.86-2.75 (m, 4H), 1.29 (d, 3H)

### Example 93

### 3-[2-(2-methoxyphenyl)ethyl]-5-(2-methoxy-(1S)-methyl-ethoxy)-N-(1-methyl-1-pyrazol-3-yl)-benzamide

The compound prepared in Example 83 (25.0 mg) was dissolved in ethyl acetate (10.0 mL) and then Pd/C (5.0 mg) was added thereto. Hydrogenation reaction was performed by stirring the solution at room temperature for 12 hours, using a hydrogen balloon. The reaction mixture was filtered with celite pad. The filtrate was concentrated under reduced pressure to obtain 15.0 mg of the titled compound in the form of yellow liquid (Yield: 59.0%).

¹H-NMR (CDCl₃) δ 8.76 (brs, 1H), 7.30-7.17 (m, 4H), 7.08-7.05 (m, 1H), 6.95 (dd, 1H), 6.89-6.83 (m, 3H), 4.61-4.53 (m, 1H), 3.83 (s, 3H), 3.75 (s, 3H), 3.60-3.47 (m, 2H), 3.41 (s, 3H), 2.93-2.82 (m, 4H), 1.30 (d, 3H)

### Examples 94 to 102

The titled compounds of Examples 94 to 102 were prepared, in accordance with the same procedures as in Example 93, using the compounds prepared in Examples 33 to 35, 38 to 41, and 46 to 47, instead of the compound prepared in Example 83, respectively.

### Example 94

### 3-[2-(2-fluorophenyl)ethyl]-5-(2-methoxy-(1S)-methyl-ethoxy)-N-(1-methyl-1-pyrazol-3-yl)-benzamide

Yield: 28.6%

¹H-NMR (CDCl₃) δ 8.45 (brs, 1H), 7.31-7.24 (m, 3H), 7.21-6.98 (m, 4H), 6.93 (s, 1H), 6.82 (d, 1H), 4.61-4.56 (m, 1H), 3.83 (s, 3H), 3.60-3.47 (m, 2H), 3.41 (s, 3H), 2.97-2.88 (m, 4H), 1.30 (d, 3H)

### Example 95

### 3-[2-(2,3-difluorophenyl)ethyl]-5-(2-methoxy-(1S)-methyl-ethoxy)-N-(1-methyl-1H-pyrazol-3-yl)-benzamide

Yield: 87.3%

¹H-NMR (CDCl₃) δ 8.56 (brs, 1H), 7.31-7.25 (m, 3H), 7.02-6.82 (m, 5H), 4.60-4.54 (m, 1H), 3.80 (s, 3H), 3.59-3.47 (m, 2H), 3.41 (s, 3H), 3.00-2.85 (m, 4H), 1.30 (d, 3H)

### Example 96

### 3-[2-(2,4-difluorophenyl)ethyl]-5-(2-methoxy-(1S)-methyl-ethoxy)-N-(1-methyl-1H-pyrazol-3-yl)-benzamide

Yield: 66.5%

¹H-NMR (CDCl₃) δ 8.57 (brs, 1H), 7.30 (d, 2H), 7.26 (s, 1H), 7.08-7.01 (m, 1H), 6.90 (s, 1H), 6.83-6.74 (m, 3H), 4.62-4.54 (m, 1H), 3.81 (s, 3H), 3.59-3.47 (m, 2H), 3.41 (s, 3H), 2.98-2.82 (m, 4H), 1.30 (d, 3H)

### Example 97

### 3-[2-(3-aminophenyl)ethyl]-5-(2-methoxy-(1S)-methyl-ethoxy)-N-(1-methyl-1-pyrazol-3-yl)-benzamide

Yield: 49.0%

¹H-NMR (CDCl₃) δ 8.54 (brs, 1H), 7.30-7.26 (m, 2H), 7.18 (s, 1H), 7.08 (t, 1H), 6.93 (t, 1H), 6.82 (d, 1H), 6.60-6.50 (m, 3H), 4.61-4.56 (m, 1H), 3.80 (s, 3H), 3.60-3.47 (m, 2H), 3.41 (s, 3H), 2.91-2.78 (m, 4H), 1.30 (d, 3H)

### Example 98

### 3-[2-(4-fluorophenyl)ethyl]-5-(2-methoxy-(1S)-methyl-ethoxy)-N-(1-methyl-1H-pyrazol-3-yl)-benzamide

Yield: 70.9%

¹H-NMR (CDCl₃) δ 8.67 (s, 1H), 7.29-7.23 (m, 3H), 7.11-7.07 (m, 2H), 6.96 (t, 2H), 6.88 (s, 1H), 6.83 (d, 1H), 4.60-4.51 (m, 1H), 3.77 (s, 3H), 3.58-3.46 (m, 2H), 3.41 (s, 3H), 2.92-2.86 (m, 4H), 1.29 (d, 3H)

### Example 99

### 3-[2-(3,5-dimethoxyphenyl)ethyl]-5-(2-methoxy-(1S)-methyl-ethoxy)-N-(1-methyl-1H-pyrazol-3-yl)-benzamide

Yield: 99.9%

¹H-NMR (CDCl₃) δ 8.59 (brs, 1H), 7.30-7.28 (m, 2H), 7.25 (s, 1H), 6.92 (s, 1H), 6.82 (s, 1H), 6.33 (brs, 3H), 4.62-4.53 (m, 1H), 3.78 (s, 3H), 3.77 (s, 6H), 3.59-3.46 (m, 2H), 3.41 (s, 3H), 2.94-2.80 (m, 4H), 1.29 (d, 3H)

### Example 100

### 3-[2-(4-methoxyphenyl)ethyl]-5-(2-methoxy-(1S)-methyl-ethoxy)-N-(1-methyl-1H-pyrazol-3-yl)-benzamide

Yield: 72.7%

¹H-NMR (CDCl₃) δ 8.52 (brs, 1H), 7.30-7.28 (m, 2H), 7.22 (s, 1H), 7.08-7.05 (m, 2H), 6.90 (t, 1H), 6.85-6.80 (m, 3H), 4.61-4.53 (m, 1H), 3.80 (s, 3H), 3.79 (s, 3H), 3.59-3.46 (m, 2H), 3.41 (s, 3H), 2.91-2.82 (m, 4H), 1.29 (d, 3H)

### Example 101

### 3-[2-(2-chlorophenyl)ethyl]-5-(2-methoxy-(1S)-methyl-ethoxy)-N-(1-methyl-1H-pyrazol-3-yl)-benzamide

Yield: 86.1%

¹H-NMR (CDCl₃) δ 8.63 (brs, 1H), 7.39-7.21 (m, 4H), 7.18-7.10 (m, 3H), 6.95 (s, 1H), 6.83 (d, 1H), 4.64-4.55 (m, 1H), 3.80 (s, 3H), 3.60-3.47 (m, 2H), 3.41 (s, 3H), 3.06-2.87 (m, 4H), 1.30 (d, 3H)

### Example 102

### 3-[2-(3-chlorophenyl)ethyl]-5-(2-methoxy-(1S)-methyl-ethoxy)-N-(1-methyl-1H-pyrazol-3-yl)-benzamide

Yield: 71.1%

¹H-NMR (CDCl₃) δ 8.59 (brs, 1H), 7.32-7.13 (m, 6H), 7.04-7.01 (m, 1H), 6.88 (t, 1H), 6.83 (d, 1H), 4.61-4.53 (m, 1H), 3.79 (s, 3H), 3.59-3.47 (m, 2H), 3.41 (s, 3H), 2.93-2.88 (m, 4H), 1.29 (d, 3H)

### Example 103

### 3-[2-(2,3-dimethoxyphenyl)ethyl]-5-(2-methoxy-(1S)-methyl-ethoxy)-N-(1-methyl-1H-pyrazol-3-yl)-benzamide

### Step 1

### 3-[trans-2-(2,3-dimethoxyphenyl)vinyl]-5-(2-methoxy-(1S)-methyl-ethoxy)-N-(1-methyl-1H-pyrazol-3-yl)-benzamide

3-[(Phosphonic acid diethyl ester)-methyl]-5-(2-methoxy-(1S)-methyl-ethoxy)-N-(1-methyl-1H-pyrazol-3-yl)-benz amide (50.0 mg) prepared in Preparation 5, potassium chloride (8.0 mg), and 18-crown-6 (9.0 mg) were dissolved in dichloromethane (1.0 mL). 2,3-Dimethoxybenzaldehyde (28.0 mg) was added to the solution, which was then stirred at room temperature for 20 hours. The reaction mixture was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/2) to obtain 32.0 mg of the titled compound in the form of yellow liquid (Yield: 52.4%).

¹H-NMR (CDCl₃) δ 8.52 (brs, 1H), 7.57 (s, 1H), 7.48 (d, 1H), 7.36 (t, 1H), 7.30 (t, 2H), 7.22 (dd, 1H), 7.11-7.05 (m, 2H), 6.88-6.83 (m, 2H), 4.69-4.62 (m, 1H), 3.89 (s, 3H), 3.87 (s, 3H), 3.82 (s, 3H), 3.64-3.51 (m, 2H), 3.43 (s, 3H), 1.36 (d, 3H)

### Step 2

### 3-[2-(2,3-dimethoxyphenyl)ethyl]-5-(2-methoxy-(1S)-methyl-ethoxy)-N-(1-methyl-1H-pyrazol-3-yl)-benzamide

3-[trans-2-(2,3-Dimethoxyphenyl)vinyl]-5-(2-methoxy-(1S)-methyl-ethoxy)-N-(1-me thyl-1H-pyrazol-3-yl)-benzamide (32.0 mg) prepared in Step 1 was dissolved in ethyl acetate (10.0 mL) and then Pd/C (5.0 mg) was added thereto. Hydrogenation reaction was performed by stirring the solution at room temperature for 12 hours, using a hydrogen balloon. The reaction mixture was filtered with celite pad. The filtrate was concentrated under reduced pressure to obtain 15.0 mg of the titled compound in the form of yellow liquid (Yield: 59.0%).

¹H-NMR (CDCl₃) δ 8.42 (brs, 1H), 7.30-7.24 (m, 3H), 7.00-6.95 (m, 2H), 6.83-6.79 (m, 2H), 6.73 (d, 1H), 4.62-4.54 (m, 1H), 3.87 (s, 3H), 3.83 (s, 3H), 3.82 (s, 3H), 3.60-3.47 (m, 2H), 3.41 (s, 3H), 2.95-2.85 (m, 4H), 1.30 (d, 3H)

### Example 104

### 3-[2-(2,4-dimethoxyphenyl)ethyl]-5-(2-methoxy-(1S)-methyl-ethoxy)-N-(1-methyl-1H-pyrazol-3-yl)-benzamide

The titled compound was prepared, in accordance with the same procedures as in Example 103, using 3-[(phosphonic acid diethyl ester)-methyl]-5-(2-methoxy-(1S)-methyl-ethoxy)-N-(1-methyl-1H-pyrazol-3-yl)-benz amide prepared in Preparation 5; and 2,4-dimethoxyaldehyde instead of 2,3-dimethoxyaldehyde.

Yield: 31.5%

¹H-NMR (CDCl₃) δ 8.43 (brs, 1H), 7.29 (t, 2H), 7.23 (s, 1H), 6.97-6.93 (m, 2H), 6.82 (d, 1H), 6.46 (d, 1H), 6.40 (dd, 1H), 4.63-4.55 (m, 1H), 3.82 (s, 3H), 3.81 (s, 3H), 3.79 (s, 3H), 3.60-3.47 (m, 2H), 3.41 (s, 3H), 2.86-2.79 (m, 4H), 1.31 (d, 3H)

### Example 105

### 3-[2-(2,4-dimethoxyphenyl)ethyl]-5-(2-methoxy-(1S)-methyl-ethoxy)-N-(pyrazin-2-yl)-benzamide

### Step 1

### 3-[2-(2,4-dimethoxyphenyl)vinyl]-5-(2-methoxy-(1S)-methyl-ethoxy)-N-(pyrazin-2-yl)-benzamide

3-[(Phosphonic acid diethyl ester)-methyl]-5-(2-methoxy-(1S)-methyl-ethoxy)-N-(pyrazin-2-yl)-benzamide (30.0 mg) prepared in Preparation 6 was tetrahydrofuran (1.0 mL). Potassium tert-butoxide (15.0 mg) and 2,4-dimethoxybenzaldehyde (9.0 μL) were was added to the solution, which was then stirred at room temperature for 12 hours. The reaction mixture was concentrated under reduced pressure. The resulting residue was dissolved in dichloromethane. The resulting solution was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/2) to obtain 19.0 mg of the titled compound in the form of yellow liquid (Yield: 65.6%).

¹H-NMR (CDCl₃) δ 9.73 (s, 1H), 8.55 (brs, 1H), 8.39 (d, 1H), 8.28 (dd, 1H), 7.60 (s, 1H), 7.49 (t, 1H), 7.43 (s, 1H), 7.36 (t, 1H), 7.29 (t, 1H), 7.00 (d, 1H), 6.53 (dd, 1H), 6.48 (d, 1H), 4.73-4.65 (m, 1H), 3.89 (s, 3H), 3.84 (s, 3H), 3.65-3.52 (m, 2H), 3.44 (s, 3H), 1.36 (d, 3H)

### Step 2

### 3-[2-(2,4-dimethoxyphenyl)ethyl]-5-(2-methoxy-(1S)-methyl-ethoxy)-N-(pyrazin-2-yl)-benzamide

3-[2-(2,4-dimethoxyphenyl)vinyl]-5-(2-methoxy-(1S)-methyl-ethoxy)-N-(pyrazin-2-yl)-benzamide (19.0 mg) prepared in Step 1 was dissolved in ethyl acetate (10.0 mL) and then Pd/C (5.0 mg) was added thereto. Hydrogenation reaction was performed by stirring the solution at room temperature for 12 hours, using a hydrogen balloon. The reaction mixture was filtered with celite pad. The filtrate was concentrated under reduced pressure to obtain 6.3 mg of the titled compound in the form of yellow liquid (Yield: 33.2%).

¹H-NMR (CDCl₃) δ 9.73 (s, 1H), 8.41-8.37 (m, 2H), 8.30-8.28 (m, 1H), 7.33 (t, 1H), 7.24 (s, 1H), 6.98 (s, 1H), 6.95 (d, 1H), 6.52-6.39 (m, 2H), 4.64-4.58 (m, 1H), 3.81 (s, 3H), 3.79 (s, 3H), 3.62-3.49 (m, 2H), 3.42 (s, 3H), 2.90-2.80 (m, 4H), 1.32 (d, 3H)

### Example 106

### 3-[2-(2-methoxyphenyl)ethyl]-5-(2-methoxy-(1S)-methyl-ethoxy)-N-(pyrazin-2-yl)-benzamide

The compound prepared in Example 48 (9.0 mg) was dissolved in ethyl acetate (0.5 mL) and then Pd/C (2.0 mg) was added thereto. Hydrogenation reaction was performed by stirring the solution at room temperature for 12 hours, using a hydrogen balloon. The reaction mixture was filtered with celite pad. The filtrate was concentrated under reduced pressure to obtain 3.0 mg of the titled compound in the form of yellow liquid (Yield: 33.9%).

¹H-NMR (CDCl₃) δ 9.70 (d, 1H), 8.40-8.38 (m, 2H), 8.29 (t, 1H), 7.34 (t, 1H), 7.24-7.18 (m, 2H), 7.07 (dd, 1H), 7.00 (s, 1H), 6.89-6.85 (m, 2H), 4.68-4.57 (m, 1H), 3.84 (s, 3H), 3.62-3.49 (m, 2H), 3.43 (s, 3H), 2.93-2.88 (m, 4H), 1.32 (d, 3H)

### Examples 107 and 108

The titled compounds of Examples 107 and 108 were prepared, in accordance with the same procedures as in Example 106, using the compounds prepared in Examples 51 and 52, instead of the compound prepared in Example 48, respectively.

### Example 107

### 3-[2-(2-fluorophenyl)ethyl]-5-(2-methoxy-(1S)-methyl-ethoxy)-N-(pyrazin-2-yl)-benz amide

Yield: 39.1%

¹H-NMR (CDCl₃) δ 9.70 (d, 1H), 8.40-8.37 (m, 2H), 8.29 (dd, 1H), 7.35 (t, 1H), 7.25 (s, 1H), 7.22-7.02 (m, 4H), 6.97 (d, 1H), 4.63-4.56 (m, 1H), 3.61-3.48 (m, 2H), 3.42 (s, 3H), 3.00-2.90 (m, 4H), 1.31 (d, 3H)

### Example 108

### 3-[2-(4-fluorophenyl)ethyl]-5-(2-methoxy-(1S)-methyl-ethoxy)-N-(pyrazin-2-yl)-benz amide

Yield: 61.1%

¹H-NMR (CDCl₃) δ 9.70 (d, 1H), 8.40-8.38 (m, 2H), 8.29 (t, 1H), 7.33 (d, 1H), 7.26 (s, 1H), 7.12-7.08 (m, 2H), 6.99-6.92 (m, 3H), 4.63-4.55 (m, 1H), 3.60-3.48 (m, 2H), 3.42 (s, 3H), 3.00-2.90 (m, 4H), 1.31 (d, 3H)

### Example 109

### 3-[2-(2,6-difluorophenyl)ethyl]-5-(2-methoxy-(1S)-methyl-ethoxy)-N-(nicotinic acid-6-yl)-benzamide

3-[2-(2,6-Difluorophenyl)ethyl]-5-(2-methoxy-(1S)-methyl-ethoxy)-N-(nicotinic acid-3-methyl ester-6-yl)-benzamide (740.0 mg) prepared in Preparation 9 was dissolved in tetrahydrofuran (3.0 mL). A solution of 1 N sodium hydroxide (2.0 mL) was added to the solution, which was then stirred at room temperature for 15 hours. The tetrahydrofuran was distilled off under reduced pressure. The resulting residue was diluted with water (30.0 mL) and then washed with dichloromethane (50.0 mL) three times. The water layer was neutralized with a 1 N hydrochloric acid solution and then extracted with dichloromethane (50.0 mL) three times. The organic layer was dried on magnesium sulfate and then concentrated under reduced pressure to obtain 191.9 mg of the titled compound in the form of white solid (Yield: 30.0%).

¹H-NMR (d₆-DMSO) δ 13.11 (brs, 1H), 11.14 (s, 1H), 8.32 (s, 2H), 7.50-7.43 (m, 2H), 7.36-7.29 (m, 1H), 7.07-7.01 (m, 2H), 6.96 (s, 1H), 4.75-4.70 (m, 1H), 3.78-3.40 (m, 2H), 3.29 (s, 3H), 2.99-2.85 (m, 4H), 2.50 (s, 3H), 1.25 (d, 3H)

### Example 110

### 3-[2-(2-aminophenyl)ethyl]-5-(2-methoxy-(1S)-methyl-ethoxy)-N-(thiazol-2-yl)-benzamide hydrochloride

The compound prepared in Example 88 (20.0 mg) was dissolved in ethyl acetate (5.0 mL). An excess of anhydrous hydrochloric acid gas was passed through the solution, which was then stirred at room temperature for 30 minutes. The reaction mixture was concentrated under reduced pressure. The resulting residue was washed with diethyl ether three times and then dried under reduced pressure to obtain 18.0 mg of the titled compound in the form of white solid (Yield: 90.3%).

¹H-NMR (CD₃OD) δ 7.79-7.75 (m, 2H), 7.53 (brs, 2H), 7.44-7.37 (m, 4H), 7.22 (s, 1H), 4.75-4.71 (m, 1H), 3.61-3.53 (m, 2H), 3.40 (s, 3H), 3.15-3.10 (m, 4H), 1.30 (d, 3H)

### Examples 111 and 112

The titled compounds of Examples 111 and 112 were prepared, in accordance with the same procedures as in Example 110, using the compounds prepared in Examples 91 and 92, instead of the compound prepared in Example 88, respectively.

### Example 111

### 3-[2-(3-aminophenyl)ethyl]-5-(2-methoxy-(1S)-methyl-ethoxy)-N-(thiazol-2-yl)-benzamide hydrochloride

Yield: 99.3%

¹H-NMR (CD₃OD) δ 7.72 (d, 1H), 7.55 (d, 1H), 7.48-7.41 (m, 3H), 7.34 (d, 1H), 7.27 (d, 1H), 7.21 (d, 1H), 7.09 (s, 1H), 4.71-4.66 (m, 1H), 3.60-3.51 (m, 2H), 3.39 (s, 3H), 3.11-3.01 (m, 4H), 1.28 (d, 3H)

### Example 112

### 3-[2-(4-aminophenyl)ethyl]-5-(2-methoxy-(1S)-methyl-ethoxy)-N-(thiazol-2-yl)-benzamide hydrochloride

Yield: 99.9%

¹H-NMR (CD₃OD) δ 7.72 (d, 1H), 7.53 (s, 1H), 7.48-7.45 (m, 2H), 7.39 (d, 2H), 7.30 (m, 2H), 7.09 (s, 1H), 4.72-4.66 (m, 1H), 3.60-3.51 (m, 2H), 3.39 (s, 3H), 3.11-3.00 (m, 4H), 1.28 (d, 3H)

### Example 113

### 3-[2-(2-methanesulfonylaminophenyl)ethyl]-5-(2-methoxy-(1S)-methyl-ethoxy)-N-(thiazol-2-yl)-benzamide

The compound prepared in Example 88 (20.0 mg) was dissolved in N,N-dimethylformamide (1.0 mL). Potassium carbonate (21.0 mg) and methanesulfonyl chloride (6 mg) were added to the solution, which was then stirred at room temperature for 12 hours. Ethyl acetate was added to the reaction mixture, which was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/1) to obtain 1.5 mg of the titled compound in the form of yellow liquid (Yield: 8.2%).

¹H-NMR (CDCl₃) δ 7.43-7.40 (m, 2H), 7.36-7.21 (m, 5H), 6.97-6.92 (m, 2H), 6.58 (s, 1H), 4.62-4.57 (m, 1H), 3.59-3.47 (m, 2H), 3.41 (s, 3H), 3.07-2.94 (m, 7H), 1.30 (d, 3H)

### Example 114

### 3-{2-[2-(2-thiophen-2-yl)acetylamino)phenyl]ethyl}-5-(2-methoxy-(1S)-methyl-ethoxy)-N-(thiazol-2-yl)-benzamide

The titled compound was prepared, in accordance with the same procedures as in Example 113, using (2-thiophen-2-yl)acetyl chloride, instead of methanesulfonyl chloride.

Yield: 29.9%

¹H-NMR (CDCl₃) δ 7.84 (d, 1H), 7.49 (s, 1H), 7.38-7.35 (m, 2H), 7.26-7.21 (m, 3H), 7.09-6.91 (m, 4H), 6.81 (s, 1H), 4.65-4.60 (m, 1H), 3.93 (s, 2H), 3.60-3.47 (m, 2H), 3.40 (s, 3H), 2.72-2.60 (m, 4H), 1.30 (d, 3H)

### Example 115

### 3-[2-(2-benzenesulfonylaminophenyl)ethyl]-5-(2-methoxy-(1S)-methyl-ethoxy)-N-(thiazol-2-yl)-benzamide

The compound prepared in Example 110 (30.0 mg) was dissolved in dichloromethane (1.0 mL). Benzenesulfonyl chloride (12 mg) and triethylamine (19.0 μL) were added to the solution, which was then stirred at room temperature for 12 hours. The reaction mixture was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/1) to obtain 13 mg of the titled compound in the form of yellow liquid (Yield: 54.1%).

¹H-NMR (CDCl₃) δ 8.16-8.14 (m, 2H), 7.92 (d, 1H), 7.73-7.41 (m, 10H), 7.12 (s, 1H), 6.63 (d, 1H), 4.63-4.57 (m, 1H), 3.63-3.49 (m, 2H), 3.43 (s, 3H), 2.78-2.65 (m, 4H), 1.35 (d, 3H)

### Examples 116 and 117

The titled compounds of Examples 116 and 117 were prepared, in accordance with the same procedures as in Example 115, using acetyl chloride and ethoxycarbonyl chloride, instead of benzenesulfonyl chloride, respectively.

### Example 116

### 3-[2-(2-acetylaminophenyl)ethyl]-5-(2-methoxy-(1S)-methyl-ethoxy)-N-(thiazol-2-yl)benzamide

Yield: 65.7%

¹H-NMR (CDCl₃) δ 7.42-7.21 (m, 6H), 7.09 (dd, 1H), 6.96 (m, 2H), 4.61-4.55 (m, 1H), 3.59-3.46 (m, 2H), 3.40 (s, 3H), 2.92-2.74 (m, 4H), 2.26 (s, 3H), 1.29 (d, 3H)

### Example 117

### 3-{2-[2-(carbamic acid ethyl ester)phenyl]ethyl}-5-(2-methoxy-(1S)-methyl-ethoxy)-N-(thiazol-2-yl)-benzamide

Yield: 27.6%

¹H-NMR (CDCl₃) δ 11.03 (brs, 1H), 7.65 (brs, 1H), 7.39 (t, 1H), 7.31-7.20 (m, 3H), 7.13-7.08 (m, 2H), 6.98 (d, 1H), 6.92 (d, 1H), 6.23 (s, 1H), 4.58-4.53 (m, 1H), 4.24-4.11 (m, 2H), 3.58-3.46 (m, 2H), 3.40 (s, 3H), 2.97-2.89 (m, 4H), 1.30-1.24 (m, 6H)

### Example 118

### 3-[2-(3-acetylaminophenyl)ethyl]-5-(2-methoxy-(1S)-methyl-ethoxy)-N-(thiazol-2-yl)-benzamide

The compound prepared in Example 111 (20.0 mg) was dissolved in N,N-dimethylformamide (1.0 mL). Potassium carbonate 19.0 mg and acetyl chloride (550 μL) were added to the solution, which was then stirred at room temperature for 12 hours. Ethyl acetate was added to the reaction mixture, which was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/1) to obtain 4.2 mg of the titled compound in the form of yellow liquid (Yield: 28.1%).

¹H-NMR (CDCl₃) δ 11.39 (brs, 1H), 7.45 (s, 1H), 7.40 (d, 2H), 7.29-7.17 (m, 4H), 6.98 (d, 2H), 6.86 (d, 1H), 4.60-4.54 (m, 1H), 3.60-3.47 (m, 2H), 3.41 (s, 3H), 2.95-2.87 (m, 4H), 2.18 (s, 3H), 1.29 (d, 3H)

### Examples 119 to 131

The titled compounds of Examples 119 to 131 were prepared, in accordance with the same procedures as in Example 118, using R″-halide, R″—NCO, R″—NCS, R″—SO₂Cl or R″—C(O)Cl suitable for Examples 119 to 131, instead of benzyl bromide, respectively.

### Example 119

### 3-[2-(3-butyrylaminophenyl)ethyl]-5-(2-methoxy-(1S)-methyl-ethoxy)-N-(thiazol-2-yl)-benzamide

Yield: 36.5%

¹H-NMR (CDCl₃) δ 11.52 (brs, 1H), 7.44-7.34 (m, 3H), 7.28-7.17 (m, 4H), 6.98 (d, 2H), 6.86 (d, 1H), 4.60-4.54 (m, 1H), 3.60-3.47 (m, 2H), 3.40 (s, 3H), 2.93-2.88 (m, 4H), 2.34 (t, 2H), 1.79-1.72 (m, 2H), 1.29 (d, 3H), 1.00 (t, 3H)

### Example 120

### 3-{2-[3-(3-methyl-butyrylamino)phenyl]ethyl}-5-(2-methoxy-(1S)-methyl-ethoxy)-N-(thiazol-2-yl)-benzamide

Yield: 20.2%

¹H-NMR (CDCl₃) δ 11.28 (brs, 1H), 7.46 (s, 1H), 7.39 (d, 1H), 7.31-7.18 (m, 5H), 6.98 (d, 2H), 6.87 (d, 1H), 4.61-4.54 (m, 1H), 3.60-3.47 (m, 2H), 3.41 (s, 3H), 2.93-2.88 (m, 4H), 2.25-2.15 (m, 3H), 1.30 (d, 3H), 1.00 (d, 6H)

### Example 121

### 3-{2-[3-(malonamic acid ethyl ester)phenyl]ethyl}-5-(2-methoxy-(1S)-methyl-ethoxy)-N-(thiazol-2-yl)-benzamide

Yield: 78.4%

¹H-NMR (CDCl₃) δ 11.65 (brs, 1H), 9.26 (s, 1H), 7.55 (s, 1H), 7.40 (s, 1H), 7.37 (s, 1H), 7.32-7.20 (m, 3H), 6.99-6.89 (m, 2H), 6.88 (d, 1H), 4.61-4.54 (m, 1H), 4.25 (q, 2H), 3.60-3.55 (m, 1H), 3.52 (s, 2H), 3.52-3.46 (m, 1H), 3.40 (s, 3H), 2.95-2.90 (m, 4H), 1.34-1.28 (m, 6H)

### Example 122

### 3-{2-[3-(carbamic acid ethyl ester)phenyl]ethyl}-5-(2-methoxy-(1S)-methyl-ethoxy)-N-(thiazol-2-yl)-benzamide

Yield: 10.6%

¹H-NMR (CDCl₃) δ 10.53 (brs, 1H), 7.38-7.25 (m, 4H), 7.22-7.12 (m, 1H), 7.13 (d, 1H), 7.00-6.97 (m, 2H), 6.84 (d, 1H), 6.64 (s, 1H), 4.62-4.55 (m, 1H), 4.23 (q, 2H), 3.60-3.47 (m, 2H), 3.41 (s, 3H), 2.96-2.90 (m, 4H), 1.32-1.28 (m, 6H)

### Example 123

### 3-{2-[3-(phenylacetylamino)phenyl]ethyl}-5-(2-methoxy-(1S)-methyl-ethoxy)-N-(thiazol-2-yl)-benzamide

Yield: 45.8%

¹H-NMR (CDCl₃) δ 11.59 (brs, 1H), 7.40-7.20 (m, 10H), 7.18-7.10 (m, 2H), 6.96 (d, 2H), 6.84 (d, 1H), 4.60-4.54 (m, 1H), 3.77 (s, 2H), 3.59-3.46 (m, 2H), 3.40 (s, 3H), 2.90-2.86 (m, 4H), 1.29 (d, 3H)

### Example 124

### 3-[2-(3-benzoylaminophenyl)ethyl]-5-(2-methoxy-(1S)-methyl-ethoxy)-N-(thiazol-2-yl)-benzamide

Yield: 8.2%

¹H-NMR (CDCl₃) δ 8.04 (s, 1H), 7.91-7.88 (m, 2H), 7.62 (s, 1H), 7.55-7.46 (m, 3H), 7.39-7.33 (m, 3H), 7.28-7.25 (m, 1H), 7.18 (s, 1H), 7.00 (d, 2H), 6.91 (d, 1H), 4.60-4.54 (m, 1H), 3.60-3.47 (m, 2H), 3.40 (s, 3H), 3.00-2.91 (m, 4H), 1.30 (d, 3H)

### Example 125

### 3-{2-[3-(4-fluorobenzoylamino)phenyl]ethyl}-5-(2-methoxy-(1S)-methyl-ethoxy)-N-(thiazol-2-yl)-benzamide

Yield: 10.2%

¹H-NMR (CDCl₃) δ 8.02 (s, 1H), 7.95-7.90 (m, 2H), 7.58 (s, 1H), 7.38-7.25 (m, 4H), 7.17-7.13 (m, 3H), 7.01-6.98 (m, 2H), 6.91 (d, 1H), 4.60-4.54 (m, 1H), 3.60-3.47 (m, 2H), 3.40 (s, 3H), 3.00-2.91 (m, 4H), 1.30 (d, 3H)

### Example 126

### 3-{2-[3-(4-chlorobenzoylamino)phenyl]ethyl}-5-(2-methoxy-(1S)-methyl-ethoxy)-N-(t hiazol-2-yl)-benzamide

Yield: 13.2%

¹H-NMR (CDCl₃) δ 8.06 (s, 1H), 7.85 (d, 2H), 7.56 (s, 1H), 7.44 (d, 2H), 7.37-7.32 (m, 3H), 7.27-7.25 (m, 1H), 7.15 (s, 1H), 7.00-6.98 (m, 2H), 6.91 (d, 1H), 4.60-4.54 (m, 1H), 3.60-3.47 (m, 2H), 3.40 (s, 3H), 3.00-2.91 (m, 4H), 1.30 (d, 3H)

### Example 127

### 3-{2-[3-(4-nitrobenzoylamino)phenyl]ethyl}-5-(2-methoxy-(1S)-methyl-ethoxy)-N-(thiazol-2-yl)-benzamide

Yield: 27.6%

¹H-NMR (CDCl₃) δ 11.37 (brs, 1H), 8.34 (s, 1H), 8.31-8.28 (m, 2H), 8.07 (d, 2H), 7.46-7.35 (m, 3H), 7.28-7.23 (m, 2H), 7.14 (s, 1H), 6.99 (d, 2H), 6.93 (d, 1H), 4.59-4.53 (m, 1H), 3.60-3.46 (m, 2H), 3.39 (s, 3H), 2.93-2.87 (m, 4H), 1.29 (d, 3H)

### Example 128

### 3-[2-(3-isonicotinamidephenyl)ethyl]-5-(2-methoxy-(1S)-methyl-ethoxy)-N-(thiazol-2-yl)-benzamide

Yield: 65.7%

¹H-NMR (CDCl₃) δ 11.81 (brs, 1H), 8.75-8.73 (m, 2H), 8.52 (s, 1H), 7.75 (d, 2H), 7.45-7.38 (m, 3H), 7.26-7.21 (m, 3H), 6.97 (d, 2H), 6.92 (d, 1H), 4.59-4.53 (m, 1H), 3.59-3.46 (m, 2H), 3.39 (s, 3H), 2.90-2.85 (m, 4H), 1.28 (d, 3H)

### Example 129

### 3-{2-[3-(2-(thiophen-2-yl)acetylamino)phenyl]ethyl}-5-(2-methoxy-(1S)-methyl-ethoxy)-N-(thiazol-2-yl)-benzamide

Yield: 70.1%

¹H-NMR (CDCl₃) δ 7.44-7.43 (m, 2H), 7.39 (s, 1H), 7.35 (s, 1H), 7.30-7.26 (m, 2H), 7.19-7.11 (m, 2H), 7.03 (d, 2H), 6.98-6.95 (m, 2H), 6.91-6.86 (m, 1H), 4.63-4.56 (m, 1H), 3.96 (s, 2H), 3.59-3.46 (m, 2H), 3.40 (s, 3H), 2.92-2.88 (m, 4H), 1.29 (d, 3H)

### Example 130

### 3-{2-[3-(3-phenyl-ureido)phenyl]ethyl}-5-(2-methoxy-(1S)-methyl-ethoxy)-N-(thiazol-2-yl)-benzamide

Yield: 12.0%

¹H-NMR (CDCl₃) δ 8.73 (s, 1H), 7.87 (s, 1H), 7.56 (s, 1H), 7.43-7.17 (m, 7H), 7.11-7.07 (m, 1H), 7.02-6.98 (m, 2H), 6.91 (s, 1H), 6.81 (d, 1H), 6.73 (d, 1H), 4.62-4.56 (m, 1H), 3.59-3.47 (m, 2H), 3.40 (s, 3H), 3.03-2.97 (m, 4H), 1.29 (d, 3H)

### Example 131

### 3-{2-[3-(3-ethyl-thio-ureido)phenyl]ethyl}-5-(2-methoxy-(1S)-methyl-ethoxy)-N-(thiazol-2-yl)-benzamide

Yield: 48.6%

¹H-NMR (CDCl₃) δ 11.62 (brs, 1H), 8.38 (s, 1H), 7.41 (d, 1H), 7.34-7.31 (m, 2H), 7.22 (d, 1H), 7.10-6.95 (m, 5H), 6.01 (s, 1H), 4.62-4.56 (m, 1H), 3.68-3.62 (m, 2H), 3.60-3.47 (m, 2H), 3.40 (s, 3H), 2.98-2.91 (m, 4H), 1.30 (d, 3H), 1.16 (t, 3H)

### Example 132

### 3-[2-(4-acetylaminophenyl)ethyl]-5-(2-methoxy-(1S)-methyl-ethoxy)-N-(thiazol-2-yl)-benzamide

The compound prepared in Example 112 (20.0 mg) was dissolved in N,N-dimethylformamide (1.0 mL). Potassium carbonate (19.0 mg) and acetyl chloride (550 μL) were added to the solution, which was then stirred at room temperature for 12 hours. Ethyl acetate was added to the reaction mixture, which was washed with distilled water and brine three times, dried on anhydrous magnesium sulfate, and then concentrated under reduced pressure to obtain a residue in the form of yellow liquid. The residue was purified with silica gel column chromatography (eluent: n-hexane/ethyl acetate=1/1) to obtain 7.4 mg of the titled compound in the form of yellow liquid (Yield: 39.7%).

¹H-NMR (CDCl₃) δ 11.28 (brs, 1H), 7.66 (s, 1H), 7.41-7.37 (m, 3H), 7.23 (d, 1H), 7.07-7.04 (m, 3H), 6.97 (d, 2H), 4.59-4.51 (m, 1H), 3.59-3.46 (m, 2H), 3.40 (s, 3H), 2.90-2.83 (m, 4H), 2.15 (s, 3H), 1.29 (d, 3H)

### Examples 133 to 146

The titled compounds of Examples 133 to 146 were prepared, in accordance with the same procedures as in Example 132, using R″-halide, R″—NCO, R″—NCS, R″—SO₂Cl or R″—C(O)Cl suitable for Examples 133 to 146, instead of benzyl bromide, respectively.

### Example 133

### 3-[2-(4-butyrylaminophenyl)ethyl]-5-(2-methoxy-(1S)-methyl-ethoxy)-N-(thiazol-2-yl)-benzamide

Yield: 24.9%

¹H-NMR (CDCl₃) δ 11.07 (brs, 1H), 7.52 (s, 1H), 7.43-7.36 (m, 3H), 7.25 (d, 1H), 7.08-7.04 (m, 3H), 6.98 (d, 2H), 4.59-4.51 (m, 1H), 3.59-3.46 (m, 2H), 3.40 (s, 3H), 2.90-2.84 (m, 4H), 2.32 (t, 2H), 1.79-1.71 (m, 2H), 1.29 (d, 3H), 0.99 (t, 3H)

### Example 134

### 3-{2-[4-(3-methyl-butyrylamino)phenyl]ethyl}-5-(2-methoxy-(1S)-methyl-ethoxy)-N-(thiazol-2-yl)-benzamide

Yield: 16.6%

¹H-NMR (CDCl₃) δ 10.62 (brs, 1H), 7.53 (s, 1H), 7.42 (d, 2H), 7.36 (t, 1H), 7.31 (d, 1H), 7.07 (d, 2H), 7.00-6.97 (m, 3H), 4.59-4.53 (m, 1H), 3.58-3.48 (m, 2H), 3.41 (s, 3H), 2.90-2.83 (m, 4H), 2.23-2.13 (m, 3H), 1.30 (d, 3H), 1.01 (d, 6H)

### Example 135

### 3-{2-[4-(malonamic acid ethyl ester)phenyl]ethyl}-5-(2-methoxy-(1S)-methyl-ethoxy)-N-(thiazol-2-yl)-benzamide

Yield: 75.2%

¹H-NMR (CDCl₃) δ 11.61 (brs, 1H), 9.20 (s, 1H), 7.45 (d, 1H), 7.39 (d, 1H), 7.30-7.18 (m, 2H), 7.10 (d, 1H), 7.00-6.90 (m, 3H), 6.62 (d, 1H), 4.59-4.53 (m, 1H), 4.25 (q, 2H), 3.59-3.45 (m, 4H), 3.40 (s, 3H), 2.91-2.85 (m, 4H), 1.33-1.24 (m, 6H)

### Example 136

### 3-{2-[4-(carbamic acid ethyl ester)phenyl]ethyl}-5-(2-methoxy-(1S)-methyl-ethoxy)-N-(thiazol-2-yl)-benzamide

Yield: 10.1%

¹H-NMR (CDCl₃) δ 10.56 (brs, 1H), 7.37-7.27 (m, 4H), 7.08-6.93 (m, 5H), 6.91 (s, 1H), 4.60-4.54 (m, 1H), 4.21 (q, 2H), 3.60-3.47 (m, 2H), 3.41 (s, 3H), 2.92-2.86 (m, 4H), 1.33-1.27 (m, 6H)

### Example 137

### 3-{2-[4-(phenylacetylamino)phenyl]ethyl}-5-(2-methoxy-(1S)-methyl-ethoxy)-N-(thiazol-2-yl)-benzamide

Yield: 23.1%

¹H-NMR (CDCl₃) δ 11.22 (brs, 1H), 7.42-7.27 (m, 9H), 7.22 (d, 1H), 7.16 (s, 1H), 7.04 (d, 2H), 6.96 (d, 2H), 4.60-4.54 (m, 1H), 3.72 (s, 2H), 3.59-3.44 (m, 2H), 3.39 (s, 3H), 2.90-2.84 (m, 4H), 1.28 (d, 3H)

### Example 138

### 3-[2-(4-benzoylaminophenyl)ethyl]-5-(2-methoxy-(1S)-methyl-ethoxy)-N-(thiazol-2-yl)-benzamide

Yield: 5.6%

¹H-NMR (CDCl₃) δ 8.39 (s, 1H), 7.90-7.87 (m, 2H), 7.57-7.48 (m, 5H), 7.34 (d, 1H), 7.30 (d, 1H), 7.13 (d, 2H), 6.99-6.97 (m, 2H), 6.77 (s, 1H), 4.62-4.56 (m, 1H), 3.61-3.48 (m, 2H), 3.41 (s, 3H), 2.93-2.87 (m, 4H), 1.31 (d, 3H)

### Example 139

### 3-{2-[4-(4-fluorobenzoylamino)phenyl]ethyl}-5-(2-methoxy-(1S)-methyl-ethoxy)-N-(t hiazol-2-yl)-benzamide

Yield: 8.7%

¹H-NMR (CDCl₃) δ 8.35 (s, 1H), 7.92-7.88 (m, 2H), 7.52 (d, 2H), 7.35 (t, 1H), 7.28-7.26 (m, 1H), 7.19-7.10 (m, 4H), 6.99 (d, 2H), 6.79 (s, 1H), 4.61-4.56 (m, 1H), 3.61-3.48 (m, 2H), 3.41 (s, 3H), 2.92-2.88 (m, 4H), 1.31 (d, 3H)

### Example 140

### 3-{2-[4-(4-chlorobenzoylamino)phenyl]ethyl}-5-(2-methoxy-(1S)-methyl-ethoxy)-N-(thiazol-2-yl)-benzamide

Yield: 11.7%

¹H-NMR (CDCl₃) δ 8.37 (s, 1H), 7.82 (d, 2H), 7.52 (d, 2H), 7.46 (d, 2H), 7.35 (d, 1H), 7.25 (d, 1H), 7.11 (d, 2H), 6.99 (d, 2H), 6.81 (s, 1H), 4.61-4.56 (m, 1H), 3.61-3.48 (m, 2H), 3.41 (s, 3H), 2.93-2.89 (m, 4H), 1.31 (d, 3H)

### Example 141

### 3-{2-[4-(3,5-dimethoxybenzoylamino)phenyl]ethyl}-5-(2-methoxy-(1S)-methyl-ethoxy)-N-(thiazol-2-yl)-benzamide

Yield: 6.9%

¹H-NMR (CDCl₃) δ 7.52-7.45 (m, 2H), 7.21-6.90 (m, 7H), 6.67 (s, 1H), 6.62 (d, 2H), 4.65-4.62 (m, 1H), 3.80 (s, 6H), 3.62-3.48 (m, 2H), 3.41 (s, 3H), 2.95-2.81 (m, 4H), 1.32 (d, 3H)

### Example 142

### 3-[2-(4-isonicotinamidephenyl)ethyl]-5-(2-methoxy-(1S)-methyl-ethoxy)-N-(thiazol-2-yl)-benzamide

Yield: 43.4%

¹H-NMR (CDCl₃) δ 8.76 (d, 2H), 8.57 (s, 1H), 7.71 (d, 2H), 7.53 (d, 2H), 7.37 (d, 1H), 7.17 (d, 1H), 7.10 (d, 2H), 6.98-6.95 (m, 3H), 4.61-4.55 (m, 1H), 3.60-3.46 (m, 2H), 3.40 (s, 3H), 2.91-2.86 (m, 4H), 1.30 (d, 3H)

### Example 143

### 3-{2-[4-(2-(thiophen-2-yl)acetylamino)phenyl]ethyl}-5-(2-methoxy-(1S)-methyl-ethoxy)-N-(thiazol-2-yl)-benzamide

Yield: 31.9%

¹H-NMR (CDCl₃) δ 11.42 (brs, 1H), 7.49 (s, 1H), 7.38-7.28 (m, 4H), 7.20-7.18 (m, 2H), 7.06-7.02 (m, 4H), 6.96 (d, 2H), 4.58-4.51 (m, 1H), 3.92 (s, 2H), 3.58-3.46 (m, 2H), 3.39 (s, 3H), 2.88-2.84 (m, 4H), 1.28 (d, 3H)

### Example 144

### 3-[2-(4-benzenesulfonylaminophenyl)ethyl]-5-(2-methoxy-(1S)-methyl-ethoxy)-N-(thiazol-2-yl)-benzamide

Yield: 6.8%

¹H-NMR (CDCl₃) δ 7.76-7.73 (m, 2H), 7.52-7.40 (m, 4H), 7.34 (s, 1H), 7.03-6.93 (m, 6H), 6.68 (s, 1H), 4.61-4.54 (m, 1H), 3.60-3.46 (m, 2H), 3.40 (s, 3H), 2.90-2.84 (m, 4H), 1.29 (d, 3H)

### Example 145

### 3-{2-[4-(3-phenyl-ureido)phenyl]ethyl}-5-(2-methoxy-(1S)-methyl-ethoxy)-N-(thiazol-2-yl)-benzamide

Yield: 26.8%

¹H-NMR (CDCl₃) δ 7.53-7.09 (m, 10H), 7.02-6.97 (m, 4H), 4.63-4.57 (m, 1H), 3.61-3.48 (m, 2H), 3.41 (s, 3H), 2.88-2.80 (m, 4H), 1.30 (d, 3H)

### Example 146

### 3-{2-[4-(3-ethyl-thio-ureido)phenyl]ethyl}-5-(2-methoxy-(1S)-methyl-ethoxy)-N-(thiazol-2-yl)-benzamide

Yield: 24.1%

¹H-NMR (CDCl₃) δ 10.63 (brs, 1H), 8.48 (s, 1H), 7.40-7.36 (m, 2H), 7.20-7.13 (m, 4H), 6.98 (d, 2H), 6.94 (s, 1H), 5.90 (s, 1H), 4.62-4.56 (m, 1H), 3.68 (q, 2H), 3.60-3.48 (m, 2H), 3.41 (s, 3H), 2.95-2.91 (m, 4H), 1.31 (d, 3H), 1.18 (t, 3H)

### Example 147

### 3-[2-(3-malonamic acid phenyl)ethyl]-5-(2-methoxy-(1S)-methyl-ethoxy)-N-(thiazol-2-yl)-benzamide

The compound prepared in Example 121 (17.0 mg) was dissolved in tetrahydrofuran (0.5 mL). A 3 N sodium hydroxide solution (0.5 mL) was added to the solution, which was then stirred at room temperature for 12 hours. The reaction mixture was concentrated under reduced pressure. The resulting water layer was neutralized with a 1 N hydrochloric acid solution to obtain a white precipitate. The precipitate was washed with distilled water three times and then dried under reduced pressure to obtain 7.1 mg of the titled compound in the form of white solid (Yield: 40.2%).

¹H-NMR (CDCl₃) δ 9.30 (s, 1H), 7.65 (s, 1H), 7.52-7.48 (m, 2H), 7.41 (d, 1H), 7.15 (t, 1H), 7.06-7.01 (m, 2H), 6.88 (d, 1H), 4.68-4.62 (m, 1H), 3.66 (s, 2H), 3.59-3.48 (m, 2H), 3.41 (s, 3H), 2.93-2.83 (m, 4H), 1.31 (d, 3H)

### Example 148

### 3-[trans-2-(4-fluorophenyl)vinyl]-5-(1-methoxymethyl-propoxy)-N-(thiazol-2-yl)-benzamide

3-[trans-2-(4-Fluorophenyl)vinyl]-5-(1-methoxymethyl-propoxy)-benzoic acid (50.0 mg) prepared in Preparation 10, HOBT (39.0 mg), EDAC (56.0 mg), triethylamine (40.0 μL), and 2-aminothiazole (29.0 mg) were added to dichloromethane (3.0 mL) and then stirred at room temperature for 12 hours. The reaction mixture was washed with a 1 N hydrochloric acid solution, a saturated sodium hydrogen carbonate solution, distilled water and brine three times, dried on anhydrous magnesium sulfate, and then concentrated under reduced pressure to obtain a residue in the form of yellow liquid residue. The residue was purified with silica gel column chromatography (eluent: n-hexane/ethyl acetate=1/1) to obtain 10.0 mg of the titled compound in the form of yellow liquid (Yield: 16.2%).

¹H-NMR (CDCl₃) δ 11.78 (brs, 1H), 7.34 (s, 1H), 7.18-6.90 (m, 9H), 6.74 (s, 1H), 4.20-4.15 (m, 1H), 3.48-3.43 (m, 2H), 3.34 (s, 3H), 1.66-1.58 (m, 2H), 0.90 (t, 3H)

### Comparative Example 1 and 2

For comparison with the compounds of the present invention, the compound having 2-(pyridyl-2-yl)vinyl group at 5-position of benzamide (Comparative Example 1) was prepared in accordance with the same procedures as in Example S 47 of WO03/000267. And also, the compound having 2-isopropyl-vinyl group at 5-position of benzamide (Comparative Example 2) was prepared in accordance with the same procedures as in Example II 91 of WO03/015774. The structures thereof are as follows:

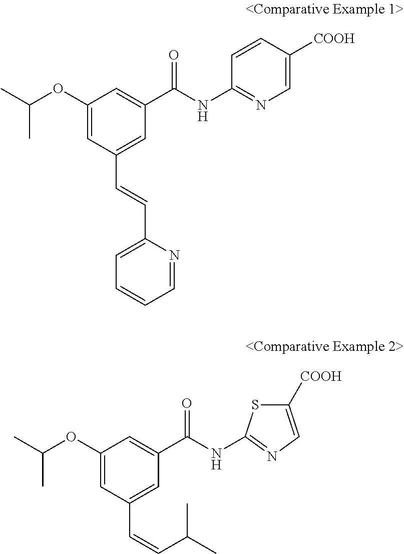

### Test Example 1

### Glucokinase Activation Assay

The amounts of glucokinase activation were determined by measuring the levels of NADPH (the final metabolite of glucose through glucokinase-mediated phospho-rylation) and then calculating to glucokinase activities.

Glucokinase (ProteinOne Inc.) was added, in the concentration of 100 ng/uL, to a enzyme-reaction medium (25 mM HEPES pH 7, 25 mM KCL, 1 mM MgCl, 5 mM D(+)-glucose, 1 mM ATP, 1 mM NADP, 1 mM DTT, and 2.5 u/mL G6PDH). Each compound in predetermined concentrations was added to the enzyme-reaction medium and then absorbance (First Absorbance) was measured at 340 nm of wavelength. After measuring the first absorbance, each reaction mixture was incubated at 24° C. for 90 minutes and then absorbance (Second Absorbance) was re-measured at 340 nm of wavelength. Using changes between First Absorbance and Second Absorbance, glucokinase activity was calculated. In the calculation, a maximum activity of the test compound (Emax), in comparison to the control group (no treatment of compound) was determined and then EC₅₀ was calculated using the software for statistical analysis “Prism”, based on the activity-changes according to concentrations of the test compounds. The results are shown in Tables 1 and 2 below.

### Test Example 2

### Analysis of Insulin Secretion in MIN6 Cells

MIN6 cells (mouse pancreatic beta cell line) were seeded, in 10⁵ cells/500 ul of concentration per well, onto a DMEM supplemented with 10% FBS in 48 well cluster. The cells were incubated at 37° C./5% CO₂ incubator for 2 days to stabilize the cells. After completing the incubation, the DMEM was discarded and the resulting cell monolayer was washed with 500 ul/well of KRB buffer (Krebs Ringer buffer: 119 mM NaCl, 4.74 mM KCl, 2.54 mM CaCl₂, 1.19 mM MgSO₄, 1.19 mM KH₂PO₄, 25 mM NaHCO₃, 10 mM HEPES pH 7.4, and 0.1% BSA). 500 ul/well of KRB buffer supplemented with 5.6 mM of glucose was added to the cells, which was then pre-incubated for 1 hour. After the pre-incubation, 500 ul/well of KRB buffer supplemented with 10 uM of test compounds and 16.8 mM of glucose was added to the cells, which were then incubated for 1 hour. Each medium was isolated and then the amount of insulin was measured as follows: The isolated medium was diluted with distilled water in a ratio of 1:20. The amount of insulin secreted to the medium was measured using Mouse Insulin ELISA immunoassay kit (Mercodia Inc.) and then % increase of insulin secretion was calculated based on the control group (no treatment of compound). The results are shown in Tables 1 and 2 below.

TABLE 1

| | | % increase | | | % increase |
|---|---|---|---|---|---|
| | EC₅₀ | of insulin | | EC₅₀ | of insulin |
| Example | (nM) | secretion | Example | (nM) | secretion |
| 1 | 164.0 | 111.7 | 2 | 172.1 | 195.4 |
| 3 | 90.2 | 92.0 | 4 | 222.5 | 102.6 |
| 5 | 172.7 | 74.3 | 6 | 38.5 | 124.8 |
| 7 | 48.4 | 97.0 | 8 | 86.4 | 75 |
| 9 | 27.5 | 139.9 | 10 | 55.2 | 94.0 |
| 11 | 30.9 | 99.8 | 12 | 14.7 | 117.3 |
| 13 | 12.7 | 141.2 | 14 | 19.4 | 138.1 |
| 15 | 127.4 | 170.5 | 16 | 8.4 | 205.5 |
| 17 | 50.4 | 89.1 | 18 | 126.2 | 89.0 |
| 19 | 52.1 | 112.6 | 20 | 123.2 | 99.1 |
| 21 | 335.6 | 174.4 | 22 | 65.6 | 76.7 |
| 23 | 41.1 | 71.3 | 24 | 47.3 | 63.2 |
| 25 | 70.2 | 49.1 | 26 | 46.5 | 81.4 |
| 27 | 86.6 | 80.4 | 28 | 117.7 | 78.3 |
| 29 | 127.6 | 111.7 | 30 | 131.9 | 125.0 |
| 31 | 47.3 | 211.8 | 32 | 283.8 | 110.4 |
| 33 | 79.8 | 84.3 | 34 | 132.0 | 101.4 |
| 35 | 109.0 | 104.6 | 36 | 200.0 | 181.1 |
| 37 | 160.6 | 170.3 | 38 | 238.9 | 116.1 |
| 39 | 119.0 | 123.2 | 40 | 173.5 | 172.7 |
| 41 | 245.1 | 130.4 | 42 | 114.6 | 197.8 |
| 43 | 322.0 | 211.8 | 44 | 168.5 | 207.9 |
| 45 | 181.0 | 185.3 | 46 | 148.8 | 198.1 |
| 47 | 107.2 | 230.5 | 48 | 281.2 | 118.7 |
| 49 | 381.4 | 163.2 | 50 | 310.7 | 111.9 |
| 51 | 238.3 | 250.5 | 52 | 419.8 | 90.8 |
| 53 | 503.2 | 294.0 | 54 | 382.1 | 165.3 |
| 55 | 346.4 | 177.2 | 56 | 300.6 | 116.8 |
| 57 | 282.6 | 243.9 | 58 | 212.5 | 300.3 |
| 59 | 377.2 | 176.9 | 60 | 397.2 | 95.4 |
| 61 | 276.4 | 98.0 | 62 | 106.2 | 98.0 |
| 63 | 402.9 | 111.3 | 64 | 159.2 | 112.8 |
| 65 | 292.1 | 76.6 | 66 | 387.5 | 106.8 |
| 67 | 230.5 | 79.5 | 68 | 355.5 | 145.7 |
| 69 | 146.2 | 175.5 | 70 | 102.2 | 83.0 |
| 71 | 84.8 | 128.0 | 72 | 120.9 | 107.0 |
| 73 | 82.8 | 154.5 | 74 | 296.3 | 96.5 |

TABLE 2

| | | % increase | | | % increase |
|---|---|---|---|---|---|
| | EC₅₀ | of insulin | | EC₅₀ | of insulin |
| Example | (nM) | secretion | Example | (nM) | secretion |
| 75 | 164.5 | 190.9 | 76 | 55.8 | 179.0 |
| 77 | 120.4 | 136.0 | 78 | 122.6 | 141.9 |
| 79 | 95.1 | 140.1 | 80 | 133.5 | 139.7 |
| 81 | 89.8 | 125.3 | 82 | 91.8 | 166.9 |
| 83 | 114.6 | 197.8 | 84 | 41.6 | 127.3 |
| 85 | 9.5 | 104.7 | 86 | 36.6 | 170.2 |
| 87 | 34.4 | 188.8 | 88 | 108.4 | 186.9 |
| 89 | 28.4 | 159.8 | 90 | 321.7 | 135.2 |
| 91 | 68.9 | 133.2 | 92 | 113.2 | 142.6 |
| 93 | 135.1 | 148.6 | 94 | 217.2 | 132.1 |
| 95 | 418.2 | 85.0 | 96 | 184.3 | 103.8 |
| 97 | 414.7 | 115.4 | 98 | 216.2 | 112.5 |
| 99 | 116.7 | 162.7 | 100 | 314.5 | 165.6 |
| 101 | 85.6 | 81.5 | 102 | 264.1 | 169.7 |
| 103 | 448.3 | 115.4 | 104 | 223.4 | 199.3 |
| 105 | 297.5 | 240.6 | 106 | 303.5 | 225.1 |
| 107 | 333.0 | 199.3 | 108 | 465.3 | 143.8 |
| 109 | 208.1 | 197.2 | 110 | 68.2 | 161.4 |
| 111 | 54.7 | 131.7 | 112 | 126.1 | 154.0 |
| 113 | 72.1 | 146.5 | 114 | 275.0 | 160.9 |
| 115 | 209.7 | 86.7 | 116 | 318.4 | 123.3 |
| 117 | 228.8 | 110.2 | 118 | 208.1 | 120.9 |
| 119 | 370.4 | 70.3 | 120 | 473.0 | 64.0 |
| 121 | 169.9 | 108.8 | 122 | 204.9 | 112.5 |
| 123 | 225.3 | 51.7 | 124 | 281.4 | 57.5 |
| 125 | 340.2 | 47.0 | 126 | 364.3 | 77.7 |
| 127 | 265.6 | 186.0 | 128 | 200.2 | 41.9 |
| 129 | 271.4 | 79.3 | 130 | 227.1 | 38.7 |
| 131 | 491.0 | 108.4 | 132 | 354.1 | 123.5 |
| 133 | 322.2 | 116.1 | 134 | 354.9 | 112.3 |
| 135 | 203.8 | 130.0 | 136 | 269.5 | 91.9 |
| 137 | 270.5 | 122.6 | 138 | 292.3 | 127.4 |
| 139 | 371.4 | 132.6 | 140 | 439.7 | 187.1 |
| 141 | 354.7 | 134.9 | 142 | 357.0 | 132.2 |
| 143 | 284.1 | 131.9 | 144 | 411.2 | 144.3 |
| 145 | 319.0 | 52.7 | 146 | 454.0 | 81.4 |
| 147 | 222.5 | 140.5 | 148 | 416.6 | 87.4 |

As shown in Tables 1 and 2, the compounds of the present invention activate glucokinase effectively, and therefore they can be usefully applied for treating glucokinase-mediated diseases, such as hyperglycemia and diabetes.

### Test Example 3

### Glucokinase Activation Assay of the Compounds of Comparative Examples

The amounts of glucokinase activation of the compounds of Comparative Examples 1 and 2 were determined, in accordance with the same procedures as in Test Example 1. The results are shown in Table 3 below.

TABLE 3

| | Comparative Example 1 | Comparative Example 2 |
|---|---|---|
| EC₅₀ (nM) | 2450.0 | 1870.0 |

1. A compound of Formula 1 or its pharmaceutically acceptable salt: 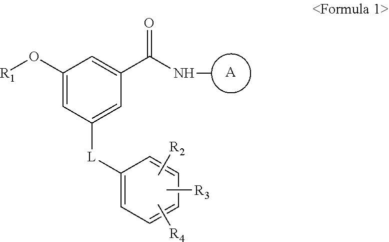
wherein,
L is —CH₂═CH₂— or —CH₂—CH₂—,
A is a heteroaryl ring having 1 to 3 hetero atoms selected from nitrogen (N) atom and sulfur (S) atom, wherein the heteroaryl ring is optionally substituted with one or more substituents selected from the group consisting of C₁-C₆ alkyl, hydroxycarbonyl, C₁-C₆ alkoxycarbonyl, and halogen,
R₁ is a C₁-C₆ alkyl group optionally substituted with C₁-C₆ alkoxy,
R₂, R₃, and R₄ is, independently each other, hydrogen; a C₁-C₆ alkyl group; a C₁-C₆ alkoxy group; halogen; nitro; amino; or —NH—R₅, with the proviso that R₂, R₃, and R₄ cannot be hydrogen at the same time,
R₅ is —C(O)—R₆, —C(O)—O—R₆, —C(O)—NH—R₆, —C(S)—NH—R₆, or —SO₂—R₆, and
R₆ is selected from the group consisting of
a C₁-C₆ alkyl group;
a C₁-C₆ alkoxycarbonyl-C₁-C₆ alkyl group;
a hydroxycarbonyl-C₁-C₆ alkyl group;
an aryl group optionally substituted with one or more substituents selected from the group consisting of nitro, halogen, and C₁-C₆ alkoxy;
an aryl-C₁-C₆ alkyl group;
a 5- or 6-membered heteroaryl ring; and
a 5- to 14-membered heteroaryl-C₁-C₆ alkyl.

2. The compound or its pharmaceutically acceptable salt of claim 1, wherein A is a heteroaryl ring selected from the group consisting of thiazolyl, pyridyl, pyrazolyl, and pyrazinyl, wherein the heteroaryl ring is optionally substituted with one or more substituents selected from the group consisting of C₁-C₆ alkyl, hydroxycarbonyl, C₁-C₆ alkoxycarbonyl, and halogen.

3. The compound or its pharmaceutically acceptable salt of claim 1, wherein R₆ is selected from the group consisting of
a C₁-C₆ alkyl group;
a C₁-C₆ alkoxycarbonyl-C₁-C₃ alkyl group;
a hydroxycarbonyl-C₁-C₃ alkyl group;
a phenyl group optionally substituted with one or more substituent selected from the group consisting of nitro, halogen, and C₁-C₃ alkoxy;
a phenyl-C₁-C₃ alkyl group;
a 5- or 6-membered heteroaryl ring; and
a 5- or 6-membered heteroaryl-C₁-C₃ alkyl.

4. The compound or its pharmaceutically acceptable salt of claim 1, which is selected from the group consisting of:
3-[trans-2-(p-tolyl)vinyl]-5-isobutoxy-N-(thiazol-2-yl)-benzamide;
3-[trans-2-(4-fluorophenyl)vinyl]-5-isobutoxy-N-(thiazol-2-yl)-benzamide;
3-[trans-2-(2-nitrophenyl)vinyl]-5-isobutoxy-N-(thiazol-2-yl)-benzamide;
3-[trans-2-(3,4-dimethoxyphenyl)vinyl]-5-isobutoxy-N-(thiazol-2-yl)-benzamide;
3-[trans-2-(2,3-dimethoxyphenyl)vinyl]-5-isobutoxy-N-(thiazol-2-yl)-benzamide;
3-[trans-2-(2-fluorophenyl)vinyl]-5-(2-methoxy-(1S)-methyl-ethoxy)-N-(thiazol-2-yl)-benzamide;
3-[trans-2-(3-fluorophenyl)vinyl]-5-(2-methoxy-(1S)-methyl-ethoxy)-N-(thiazol-2-yl)-benzamide;
3-[trans-2-(4-fluorophenyl)vinyl]-5-(2-methoxy-(1S)-methyl-ethoxy)-N-(thiazol-2-yl)-benzamide;
3-[trans-2-(2,3-difluorophenyl)vinyl]-5-(2-methoxy-(1S)-methyl-ethoxy)-N-(thiazol-2-yl)-benzamide;
3-[trans-2-(2,4-difluorophenyl)vinyl]-5-(2-methoxy-(1S)-methyl-ethoxy)-N-(thiazol-2-yl)-benzamide;
3-[trans-2-(2,5-difluorophenyl)vinyl]-5-(2-methoxy-(1S)-methyl-ethoxy)-N-(thiazol-2-yl)-benzamide;
3-[trans-2-(2,6-difluorophenyl)vinyl]-5-(2-methoxy-(1S)-methyl-ethoxy)-N-(thiazol-2-yl)-benzamide;
3-[trans-2-(2-nitrophenyl)vinyl]-5-(2-methoxy-(1S)-methyl-ethoxy)-N-(thiazol-2-yl)-benzamide;
3-[trans-2-(3-nitrophenyl)vinyl]-5-(2-methoxy-(1S)-methyl-ethoxy)-N-(thiazol-2-yl)-benzamide;
3-[trans-2-(4-nitrophenyl)vinyl]-5-(2-methoxy-(1S)-methyl-ethoxy)-N-(thiazol-2-yl)-benzamide;
3-[trans-2-(2-methoxyphenyl)vinyl]-5-(2-methoxy-(1S)-methyl-ethoxy)-N-(thiazol-2-yl)-benzamide;
3-[trans-2-(2,3-dimethoxyphenyl)vinyl]-5-(2-methoxy-(1S)-methyl-ethoxy)-N-(thiazol-2-yl)-benzamide;
3-[trans-2-(2,3,5-trifluorophenyl)vinyl]-5-(2-methoxy-(1S)-methyl-ethoxy)-N-(thiazol-2-yl)-benzamide;
3-[trans-2-(2-nitrophenyl)vinyl]-5-(2-methoxy-(1S)-methyl-ethoxy)-N-(5-fluorothiazol-2-yl)-benzamide;
3-[trans-2-(3-nitrophenyl)vinyl]-5-(2-methoxy-(1S)-methyl-ethoxy)-N-(5-fluorothiazol-2-yl)-benzamide;
3-[trans-2-(4-nitrophenyl)vinyl]-5-(2-methoxy-(1S)-methyl-ethoxy)-N-(5-fluorothiazol-2-yl)-benzamide;
3-[trans-2-(2-fluorophenyl)vinyl]-5-(2-methoxy-(1S)-methyl-ethoxy)-N-(5-fluorothiazol-2-yl)-benzamide;
3-[trans-2-(4-fluorophenyl)vinyl]-5-(2-methoxy-(1S)-methyl-ethoxy)-N-(5-fluorothiazol-2-yl)-benzamide;
3-[trans-2-(2,3-difluorophenyl)vinyl]-5-(2-methoxy-(1S)-methyl-ethoxy)-N-(5-fluorothiazol-2-yl)-benzamide;
3-[trans-2-(2,4-difluorophenyl)vinyl]-5-(2-methoxy-(1S)-methyl-ethoxy)-N-(5-fluorothiazol-2-yl)-benzamide;
3-[trans-2-(2,5-difluorophenyl)vinyl]-5-(2-methoxy-(1S)-methyl-ethoxy)-N-(5-fluorothiazol-2-yl)-benzamide;
3-[trans-2-(2,3,5-trifluorophenyl)vinyl]-5-(2-methoxy-(1S)-methyl-ethoxy)-N-(5-fluorothiazol-2-yl)-benzamide;
3-[trans-2-(2,3-dimethoxyphenyl)vinyl]-5-(2-methoxy-(1S)-methyl-ethoxy)-N-(5-fluorothiazol-2-yl)-benzamide;
3-[trans-2-(2,6-dimethoxyphenyl)vinyl]-5-(2-methoxy-(1S)-methyl-ethoxy)-N-(5-fluorothiazol-2-yl)-benzamide;
3-[trans-2-(3,4-dimethoxyphenyl)vinyl]-5-(2-methoxy-(1S)-methyl-ethoxy)-N-(5-fluorothiazol-2-yl)-benzamide;
3-[trans-2-(3,5-dimethoxyphenyl)vinyl]-5-(2-methoxy-(1S)-methyl-ethoxy)-N-(5-fluorothiazol-2-yl)-benzamide;
3-[trans-2-(2-nitrophenyl)vinyl]-5-(2-methoxy-(1S)-methyl-ethoxy)-N-(1-methyl-1H-pyrazol-3-yl)-benzamide;
3-[trans-2-(2-fluorophenyl)vinyl]-5-(2-methoxy-(1S)-methyl-ethoxy)-N-(1-methyl-1H-pyrazol-3-yl)-benzamide;
3-[trans-2-(2,3-difluorophenyl)vinyl]-5-(2-methoxy-(1S)-methyl-ethoxy)-N-(1-methyl-1H-pyrazol-3-yl)-benzamide;
3-[trans-2-(2,4-difluorophenyl)vinyl]-5-(2-methoxy-(1S)-methyl-ethoxy)-N-(1-methyl-1H-pyrazol-3-yl)-benzamide;
3-[trans-2-(2,5-difluorophenyl)vinyl]-5-(2-methoxy-(1S)-methyl-ethoxy)-N-(1-methyl-1H-pyrazol-3-yl)-benzamide;
3-[trans-2-(2,6-difluorophenyl)vinyl]-5-(2-methoxy-(1S)-methyl-ethoxy)-N-(1-methyl-1H-pyrazol-3-yl)-benzamide;
3-[trans-2-(3-nitrophenyl)vinyl]-5-(2-methoxy-(1S)-methyl-ethoxy)-N-(1-methyl-1H-pyrazol-3-yl)-benzamide;
3-[trans-2-(4-fluorophenyl)vinyl]-5-(2-methoxy-(1S)-methyl-ethoxy)-N-(1-methyl-1H-pyrazol-3-yl)-benzamide;
3-[trans-2-(3,5-dimethoxyphenyl)vinyl]-5-(2-methoxy-(1S)-methyl-ethoxy)-N-(1-methyl-1H-pyrazol-3-yl)-benzamide;
3-[trans-2-(4-methoxyphenyl)vinyl]-5-(2-methoxy-(1S)-methyl-ethoxy)-N-(1-methyl-1H-pyrazol-3-yl)-benzamide;
3-[trans-2-(2-methoxyphenyl)vinyl]-5-(2-methoxy-(1S)-methyl-ethoxy)-N-(1-methyl-1H-pyrazol-3-yl)-benzamide;
3-[trans-2-(2-bromophenyl)vinyl]-5-(2-methoxy-(1S)-methyl-ethoxy)-N-(1-methyl-1H-pyrazol-3-yl)-benzamide;
3-[trans-2-(3-bromophenyl)vinyl]-5-(2-methoxy-(1S)-methyl-ethoxy)-N-(1-methyl-1H-pyrazol-3-yl)-benzamide;
3-[trans-2-(4-bromophenyl)vinyl]-5-(2-methoxy-(1S)-methyl-ethoxy)-N-(1-methyl-1H-pyrazol-3-yl)-benzamide;
3-[trans-2-(2-chlorophenyl)vinyl]-5-(2-methoxy-(1S)-methyl-ethoxy)-N-(1-methyl-1H-pyrazol-3-yl)-benzamide;
3-[trans-2-(3-chlorophenyl)vinyl]-5-(2-methoxy-(1S)-methyl-ethoxy)-N-(1-methyl-1H-pyrazol-3-yl)-benzamide;
3-[trans-2-(2-methoxyphenyl)vinyl]-5-(2-methoxy-(1S)-methyl-ethoxy)-N-(pyrazin-2-yl)-benzamide;
3-[trans-2-(4-methoxyphenyl)vinyl]-5-(2-methoxy-(1S)-methyl-ethoxy)-N-(pyrazin-2-yl)-benzamide;
3-[trans-2-(3,5-dimethoxyphenyl)vinyl]-5-(2-methoxy-(1S)-methyl-ethoxy)-N-(pyrazin-2-yl)-benzamide;
3-[trans-2-(2-fluorophenyl)vinyl]-5-(2-methoxy-(1S)-methyl-ethoxy)-N-(pyrazin-2-yl)-benzamide;
3-[trans-2-(4-fluorophenyl)vinyl]-5-(2-methoxy-(1S)-methyl-ethoxy)-N-(pyrazin-2-yl)-benzamide;
3-[trans-2-(2-chlorophenyl)vinyl]-5-(2-methoxy-(1S)-methyl-ethoxy)-N-(pyrazin-2-yl)-benzamide;
3-[trans-2-(3-chlorophenyl)vinyl]-5-(2-methoxy-(1S)-methyl-ethoxy)-N-(pyrazin-2-yl)-benzamide;
3-[trans-2-(2,3-difluorophenyl)vinyl]-5-(2-methoxy-(1S)-methyl-ethoxy)-N-(pyrazin-2-yl)-benzamide;
3-[trans-2-(2,4-difluorophenyl)vinyl]-5-(2-methoxy-(1S)-methyl-ethoxy)-N-(pyrazin-2-yl)-benzamide;
3-[trans-2-(2,5-difluorophenyl)vinyl]-5-(2-methoxy-(1S)-methyl-ethoxy)-N-(pyrazin-2-yl)-benzamide;
3-[trans-2-(2,6-difluorophenyl)vinyl]-5-(2-methoxy-(1S)-methyl-ethoxy)-N-(pyrazin-2-yl)-benzamide;
3-[trans-2-(3-bromophenyl)vinyl]-5-(2-methoxy-(1S)-methyl-ethoxy)-N-(pyrazin-2-yl)-benzamide;
3-[trans-2-(4-bromophenyl)vinyl]-5-(2-methoxy-(1S)-methyl-ethoxy)-N-(pyrazin-2-yl)-benzamide;
3-[trans-2-(2-methoxyphenyl)vinyl]-5-(2-methoxy-(1S)-methyl-ethoxy)-N-(nicotinic acid-3-methyl ester-6-yl)-benzamide;
3-[trans-2-(2-nitrophenyl)vinyl]-5-(2-methoxy-(1S)-methyl-ethoxy)-N-(nicotinic acid-3-methyl ester-6-yl)-benzamide;
3-[trans-2-(3-nitrophenyl)vinyl]-5-(2-methoxy-(1S)-methyl-ethoxy)-N-(nicotinic acid-3-methyl ester-6-yl)-benzamide;
3-[trans-2-(2-fluorophenyl)vinyl]-5-(2-methoxy-(1S)-methyl-ethoxy)-N-(nicotinic acid-3-methyl ester-6-yl)-benzamide;
3-[trans-2-(4-fluorophenyl)vinyl]-5-(2-methoxy-(1S)-methyl-ethoxy)-N-(nicotinic acid-3-methyl ester-6-yl)-benzamide;
3-[trans-2-(2,3-difluorophenyl)vinyl]-5-(2-methoxy-(1S)-methyl-ethoxy)-N-(nicotinic acid-3-methyl ester-6-yl)-benzamide;
3-[trans-2-(2,4-difluorophenyl)vinyl]-5-(2-methoxy-(1S)-methyl-ethoxy)-N-(nicotinic acid-3-methyl ester-6-yl)-benzamide;
3-[trans-2-(2,5-difluorophenyl)vinyl]-5-(2-methoxy-(1S)-methyl-ethoxy)-N-(nicotinic acid-3-methyl ester-6-yl)-benzamide;
3-[trans-2-(2,6-difluorophenyl)vinyl]-5-(2-methoxy-(1S)-methyl-ethoxy)-N-(nicotinic acid-3-methyl ester-6-yl)-benzamide;
3-[trans-2-(2-fluorophenyl)vinyl]-5-isobutoxy-N-(thiazol-2-yl)-benzamide;
3-[trans-2-(2,5-difluorophenyl)vinyl]-5-isobutoxy-N-(thiazol-2-yl)-benzamide;
3-[trans-2-(2,6-difluorophenyl)vinyl]-5-isobutoxy-N-(thiazol-2-yl)-benzamide;
3-[trans-2-(2-methoxyphenyl)vinyl]-5-(2-methoxy-(1S)-methyl-ethoxy)-N-(nicotinic acid-6-yl)-benzamide;
3-[trans-2-(2-nitrophenyl)vinyl]-5-(2-methoxy-(1S)-methyl-ethoxy)-N-(nicotinic acid-6-yl)-benzamide;
3-[trans-2-(3-nitrophenyl)vinyl]-5-(2-methoxy-(1S)-methyl-ethoxy)-N-(nicotinic acid-6-yl)-benzamide;
3-[trans-2-(2-fluorophenyl)vinyl]-5-(2-methoxy-(1S)-methyl-ethoxy)-N-(nicotinic acid-6-yl)-benzamide;
3-[trans-2-(4-fluorophenyl)vinyl]-5-(2-methoxy-(1S)-methyl-ethoxy)-N-(nicotinic acid-6-yl)-benzamide;
3-[trans-2-(2,3-difluorophenyl)vinyl]-5-(2-methoxy-(1S)-methyl-ethoxy)-N-(nicotinic acid-6-yl)-benzamide;
3-[trans-2-(2,4-difluorophenyl)vinyl]-5-(2-methoxy-(1S)-methyl-ethoxy)-N-(nicotinic acid-6-yl)-benzamide;
3-[trans-2-(2,5-difluorophenyl)vinyl]-5-(2-methoxy-(1S)-methyl-ethoxy)-N-(nicotinic acid-6-yl)-benzamide;
3-[trans-2-(2,6-difluorophenyl)vinyl]-5-(2-methoxy-(1S)-methyl-ethoxy)-N-(nicotinic acid-6-yl)-benzamide;
3-[trans-2-(2-methoxyphenyl)vinyl]-5-(2-methoxy-(1S)-methyl-ethoxy)-N-(5-fluorothiazol-2-yl)-benzamide;
3-[trans-2-(2-methoxyphenyl)vinyl]-5-(2-methoxy-(1S)-methyl-ethoxy)-N-(1-methyl-1H-pyrazol-3-yl)-benzamide;
3-[2-(2-fluorophenyl)ethyl]-5-(2-methoxy-(1S)-methyl-ethoxy)-N-(thiazol-2-yl)-benzamide;
3-[2-(2,4-difluorophenyl)ethyl]-5-(2-methoxy-(1S)-methyl-ethoxy)-N-(thiazol-2-yl)-benzamide;
3-[2-(2,5-difluorophenyl)ethyl]-5-(2-methoxy-(1S)-methyl-ethoxy)-N-(thiazol-2-yl)-benzamide;
3-[2-(2,6-difluorophenyl)ethyl]-5-(2-methoxy-(1S)-methyl-ethoxy)-N-(thiazol-2-yl)-benzamide;
3-[2-(2-aminophenyl)ethyl]-5-(2-methoxy-(1S)-methyl-ethoxy)-N-(thiazol-2-yl)-benzamide;
3-[2-(2-methoxyphenyl)ethyl]-5-(2-methoxy-(1S)-methyl-ethoxy)-N-(thiazol-2-yl)-benzamide;
3-[2-(2,3-dimethoxyphenyl)ethyl]-5-(2-methoxy-(1S)-methyl-ethoxy)-N-(thiazol-2-yl)-benzamide;
3-[2-(3-aminophenyl)ethyl]-5-(2-methoxy-(1S)-methyl-ethoxy)-N-(thiazol-2-yl)-benzamide;
3-[2-(4-aminophenyl)ethyl]-5-(2-methoxy-(1S)-methyl-ethoxy)-N-(thiazol-2-yl)-benzamide;
3-[2-(2-methoxyphenyl)ethyl]-5-(2-methoxy-(1S)-methyl-ethoxy)-N-(1-methyl-1H-pyrazol-3-yl)-benzamide;
3-[2-(2-fluorophenyl)ethyl]-5-(2-methoxy-(1S)-methyl-ethoxy)-N-(1-methyl-1H-pyrazol-3-yl)-benzamide;
3-[2-(2,3-difluorophenyl)ethyl]-5-(2-methoxy-(1S)-methyl-ethoxy)-N-(1-methyl-1H-pyrazol-3-yl)-benzamide;
3-[2-(2,4-difluorophenyl)ethyl]-5-(2-methoxy-(1S)-methyl-ethoxy)-N-(1-methyl-1H-pyrazol-3-yl)-benzamide;
3-[2-(3-aminophenyl)ethyl]-5-(2-methoxy-(1S)-methyl-ethoxy)-N-(1-methyl-1H-pyrazol-3-yl)-benzamide;
3-[2-(4-fluorophenyl)ethyl]-5-(2-methoxy-(1S)-methyl-ethoxy)-N-(1-methyl-1H-pyrazol-3-yl)-benzamide;
3-[2-(3,5-dimethoxyphenyl)ethyl]-5-(2-methoxy-(1S)-methyl-ethoxy)-N-(1-methyl-1H-pyrazol-3-yl)-benzamide;
3-[2-(4-methoxyphenyl)ethyl]-5-(2-methoxy-(1S)-methyl-ethoxy)-N-(1-methyl-1H-pyrazol-3-yl)-benzamide;
3-[2-(2-chlorophenyl)ethyl]-5-(2-methoxy-(1S)-methyl-ethoxy)-N-(1-methyl-1H-pyrazol-3-yl)-benzamide;
3-[2-(3-chlorophenyl)ethyl]-5-(2-methoxy-(1S)-methyl-ethoxy)-N-(1-methyl-1H-pyrazol-3-yl)-benzamide;
3-[2-(2,3-dimethoxyphenyl)ethyl]-5-(2-methoxy-(1S)-methyl-ethoxy)-N-(1-methyl-1H-pyrazol-3-yl)-benzamide;
3-[2-(2,4-dimethoxyphenyl)ethyl]-5-(2-methoxy-(1S)-methyl-ethoxy)-N-(1-methyl-1H-pyrazol-3-yl)-benzamide;
3-[2-(2,4-dimethoxyphenyl)ethyl]-5-(2-methoxy-(1S)-methyl-ethoxy)-N-(pyrazin-2-yl)-benzamide;
3-[2-(2-methoxyphenyl)ethyl]-5-(2-methoxy-(1S)-methyl-ethoxy)-N-(pyrazin-2-yl)-benzamide;
3-[2-(2-fluorophenyl)ethyl]-5-(2-methoxy-(1S)-methyl-ethoxy)-N-(pyrazin-2-yl)-benzamide;
3-[2-(4-fluorophenyl)ethyl]-5-(2-methoxy-(1S)-methyl-ethoxy)-N-(pyrazin-2-yl)-benzamide;
3-[2-(2,6-difluorophenyl)ethyl]-5-(2-methoxy-(1S)-methyl-ethoxy)-N-(nicotinic acid-6-yl)-benzamide;
3-[2-(2-aminophenyl)ethyl]-5-(2-methoxy-(1S)-methyl-ethoxy)-N-(thiazol-2-yl)-benzamide hydrochloride;
3-[2-(3-aminophenyl)ethyl]-5-(2-methoxy-(1S)-methyl-ethoxy)-N-(thiazol-2-yl)-benzamide hydrochloride;
3-[2-(4-aminophenyl)ethyl]-5-(2-methoxy-(1S)-methyl-ethoxy)-N-(thiazol-2-yl)-benzamide hydrochloride;
3-[2-(2-methanesulfonylaminophenyl)ethyl]-5-(2-methoxy-(1S)-methyl-ethoxy)-N-(thiazol-2-yl)-benzamide;
3-{2-[2-(2-thiophen-2-yl)acetylamino)phenyl]ethyl}-5-(2-methoxy-(1S)-methyl-ethoxy)-N-(thiazol-2-yl)-benzamide;
3-[2-(2-benzenesulfonylaminophenyl)ethyl]-5-(2-methoxy-(1S)-methyl-ethoxy)-N-(thiazol-2-yl)-benzamide;
3-[2-(2-acetylaminophenyl)ethyl]-5-(2-methoxy-(1S)-methyl-ethoxy)-N-(thiazol-2-yl)-benzamide;
3-{2-[2-(carbamic acid ethyl ester)phenyl]ethyl}-5-(2-methoxy-(1S)-methyl-ethoxy)-N-(thiazol-2-yl)-benzamide;
3-[2-(3-acetylaminophenyl)ethyl]-5-(2-methoxy-(1S)-methyl-ethoxy)-N-(thiazol-2-yl)-benzamide;
3-[2-(3-butyrylaminophenyl)ethyl]-5-(2-methoxy-(1S)-methyl-ethoxy)-N-(thiazol-2-yl)-benzamide;
3-{2-[3-(3-methyl-butyrylamino)phenyl]ethyl}-5-(2-methoxy-(1S)-methyl-ethoxy)-N-(thiazol-2-yl)-benzamide;
3-{2-[3-(malonamic acid ethyl ester)phenyl]ethyl}-5-(2-methoxy-(1S)-methyl-ethoxy)-N-(thiazol-2-yl)-benzamide;
3-{2-[3-(carbamic acid ethyl ester)phenyl]ethyl}-5-(2-methoxy-(1S)-methyl-ethoxy)-N-(thiazol-2-yl)-benzamide;
3-{2-[3-(phenylacetylamino)phenyl]ethyl}-5-(2-methoxy-(1S)-methyl-ethoxy)-N-(thiazol-2-yl)-benzamide;
3-[2-(3-benzoylaminophenyl)ethyl]-5-(2-methoxy-(1S)-methyl-ethoxy)-N-(thiazol-2-yl)-benzamide;
3-{2-[3-(4-fluorobenzoylamino)phenyl]ethyl}-5-(2-methoxy-(1S)-methyl-ethoxy)-N-(thiazol-2-yl)-benzamide;
3-{2-[3-(4-chlorobenzoylamino)phenyl]ethyl}-5-(2-methoxy-(1S)-methyl-ethoxy)-N-(thiazol-2-yl)-benzamide;
3-{2-[3-(4-nitrobenzoylamino)phenyl]ethyl}-5-(2-methoxy-(1S)-methyl-ethoxy)-N-(thiazol-2-yl)-benzamide;
3-[2-(3-isonicotinamidephenyl)ethyl]-5-(2-methoxy-(1S)-methyl-ethoxy)-N-(thiazol-2-yl)-benzamide;
3-{2-[3-(2-(thiophen-2-yl)acetylamino)phenyl]ethyl}-5-(2-methoxy-(1S)-methyl-ethoxy)-N-(thiazol-2-yl)-benzamide;
3-{2-[3-(3-phenyl-ureido)phenyl]ethyl}-5-(2-methoxy-(1S)-methyl-ethoxy)-N-(thiazol-2-yl)-benzamide;
3-{2-[3-(3-ethyl-thio-ureido)phenyl]ethyl}-5-(2-methoxy-(1S)-methyl-ethoxy)-N-(thiazol-2-yl)-benzamide;
3-[2-(4-acetylaminophenyl)ethyl]-5-(2-methoxy-(1S)-methyl-ethoxy)-N-(thiazol-2-yl)-benzamide;
3-[2-(4-butyrylaminophenyl)ethyl]-5-(2-methoxy-(1S)-methyl-ethoxy)-N-(thiazol-2-yl)-benzamide;
3-{2-[4-(3-methyl-butyrylamino)phenyl]ethyl}-5-(2-methoxy-(1S)-methyl-ethoxy)-N-(thiazol-2-yl)-benzamide;
3-{2-[4-(malonamic acid ethyl ester)phenyl]ethyl}-5-(2-methoxy-(1S)-methyl-ethoxy)-N-(thiazol-2-yl)-benzamide;
3-{2-[4-(carbamic acid ethyl ester)phenyl]ethyl}-5-(2-methoxy-(1S)-methyl-ethoxy)-N-(thiazol-2-yl)-benzamide;
3-{2-[4-(phenylacetylamino)phenyl]ethyl}-5-(2-methoxy-(1S)-methyl-ethoxy)-N-(thiazol-2-yl)-benzamide;
3-[2-(4-benzoylaminophenyl)ethyl]-5-(2-methoxy-(1S)-methyl-ethoxy)-N-(thiazol-2-yl)-benzamide;
3-{2-[4-(4-fluorobenzoylamino)phenyl]ethyl}-5-(2-methoxy-(1S)-methyl-ethoxy)-N-(thiazol-2-yl)-benzamide;
3-{2-[4-(4-chlorobenzoylamino)phenyl]ethyl}-5-(2-methoxy-(1S)-methyl-ethoxy)-N-(thiazol-2-yl)-benzamide;
3-{2-[4-(3,5-dimethoxybenzoylamino)phenyl]ethyl}-5-(2-methoxy-(1S)-methyl-ethoxy)-N-(thiazol-2-yl)-benzamide;
3-[2-(4-isonicotinamidephenyl)ethyl]-5-(2-methoxy-(1S)-methyl-ethoxy)-N-(thiazol-2-yl)-benzamide;
3-{2-[4-(2-(thiophen-2-yl)acetylamino)phenyl]ethyl}-5-(2-methoxy-(1S)-methyl-ethoxy)-N-(thiazol-2-yl)-benzamide;
3-[2-(4-benzenesulfonylaminophenyl)ethyl]-5-(2-methoxy-(1S)-methyl-ethoxy)-N-(thiazol-2-yl)-benzamide;
3-{2-[4-(3-phenyl-ureido)phenyl]ethyl}-5-(2-methoxy-(1S)-methyl-ethoxy)-N-(thiazol-2-yl)-benzamide;
3-{2-[4-(3-ethyl-thio-ureido)phenyl]ethyl}-5-(2-methoxy-(1S)-methyl-ethoxy)-N-(thiazol-2-yl)-benzamide;
3-[2-(3-malonamic acid phenyl)ethyl]-5-(2-methoxy-(1S)-methyl-ethoxy)-N-(thiazol-2-yl)-benzamide; and
3-[trans-2-(4-fluorophenyl)vinyl]-5-(1-methoxymethyl-propoxy)-N-(thiazol-2-yl)-benzamide.

5. A process for preparing a compound of Formula 1a or its pharmaceutically acceptable salt, which comprises reacting a compound of Formula 2 with a compound of Formula 3: 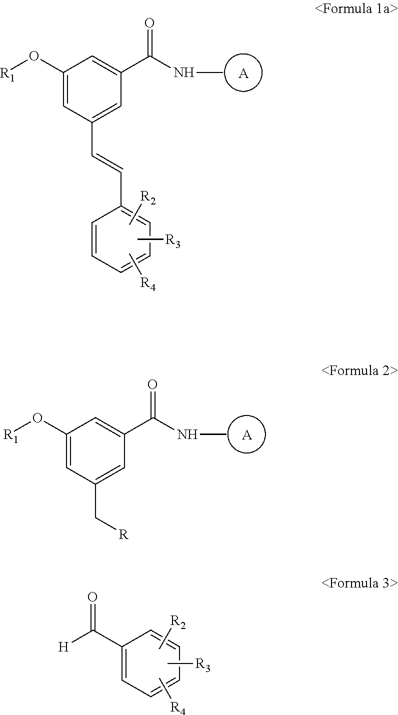
wherein, A, R₁, R₂, R₃, and R₄ are the same as defined in claim 1; R is —P(O)(OR′)₂ or triphenylphosphonium (—PPh₃); and R′ is a C₁-C₆ alkyl group or an aryl group.

6. A process for preparing a compound of Formula 1a or its pharmaceutically acceptable salt, which comprises reacting a compound of Formula 4 with a compound of Formula 5: 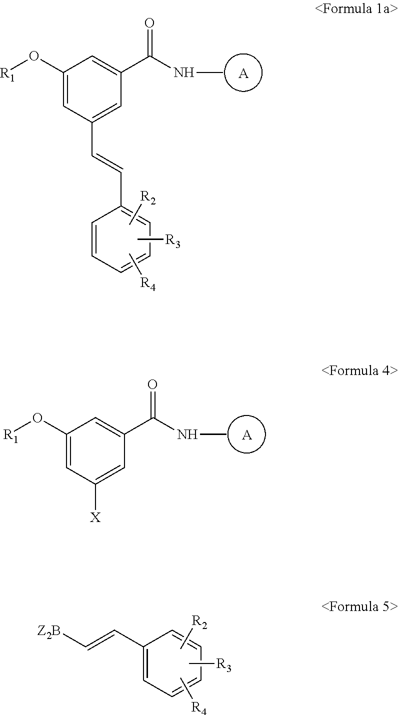
wherein, A, R₁, R₂, R₃, and R₄ are the same as defined in claim 1; X is halogen, O-trifluoromethanesulfonyl, or —OP(O)(OR′)₂; R′ is a C₁-C₆ alkyl group or an aryl group; and Z is hydroxy, a C₁-C₆ alkyl group, or a O—C₁-C₆ alkyl group.

7. A process for preparing a compound of Formula 1b or its pharmaceutically acceptable salt, which comprises reducing a compound of Formula 1a: 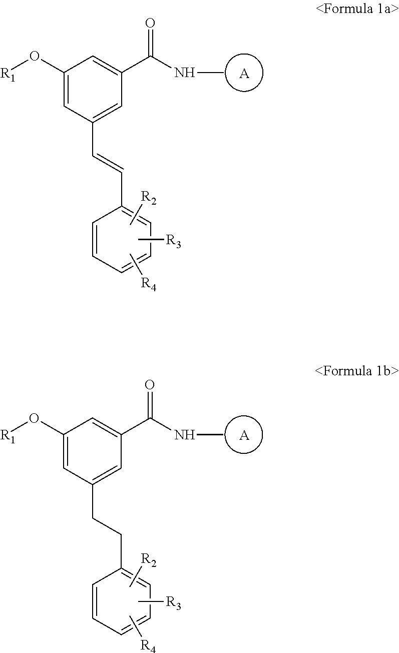
wherein, A, R₁, R₂, R₃, and R₄ are the same as defined in claim 1.

8. A compound of Formula 2: 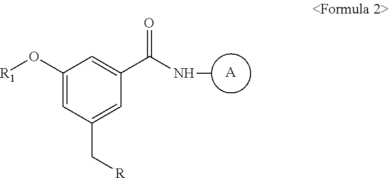
wherein, A and R₁ are the same as defined in claim 1; R is —P(O)(OR′)₂ or tri-phenylphosphonium (—PPh₃); and R′ is a C₁-C₆ alkyl group or an aryl group.

9. A pharmaceutical composition for preventing or treating a glucokinase-mediated disease comprising a therapeutically effective amount of the compound of Formula 1 or its pharmaceutically acceptable salt of claim 1; and a pharmaceutically acceptable carrier.
